(19) United States
(12) Patent Application Publication
(10) Pub. No.: US 2024/0309401 A1
(43) Pub. Date: Sep. 19, 2024

(54) METHOD FOR SILENCING GENES

(71) Applicant: Tropic Biosciences UK Limited, Norwich, Norfolk (GB)

(72) Inventors: Eyal MAORI, Cambridge (GB); Ofir MEIR, Norwich (GB)

(73) Assignee: Tropic Biosciences UK Limited, Norwich, Norfolk (GB)

(21) Appl. No.: 18/281,163

(22) PCT Filed: Mar. 8, 2022
(86) PCT No.: PCT/GB2022/050599

§ 371 Date: Sep. 8, 2023

(30) Foreign Application Priority Data
Mar. 9, 2021 (GB) 2103256.0

(51) Int. Cl. C12N 15/87; C12N 15/11; C12N 15/82

(52) U.S. Cl. CPC C12N 15/87; C12N 15/111; C12N 15/8205; C12N 2310/141; C12N 2330/50

(57) ABSTRACT

The present invention relates to improved methods and products for silencing expression of target genes in eukaryotic cells.

### FIELD OF THE INVENTION

The present invention relates to improved methods and products for silencing expression of target genes in eukaryotic cells.

### BACKGROUND OF THE INVENTION

The ability to silence the expression of specific genes provides great opportunities in a range of different fields, including research tools, therapeutics and agriculture. In relation to agriculture, improved crops are required so that the global food supply can keep up with continuing population growth. There is a limit to the traits that can be generated using traditional breeding, and this is particularly true for certain crops, so silencing genes using genetic manipulation presents a promising approach.

Silencing of genes in plants and other eukaryotes is routinely achieved using transgenic RNAi. Generation of an RNAi silencing construct requires designing a sequence that encodes a silencing RNA, such as a double stranded hairpin construct. Specificity for the target gene of interest is achieved by using a sequence with complementarity to the target gene of interest. The sequence encoding the silencing RNA is then combined with an independent promoter and a terminator that drive strong expression. In order to reduce exogenous sequence, a promoter derived from the target organism may be used and fused to the sequencing construct (e.g. Miroshnichenko et al., 2020, PCTOC, 140:691-705).

WO 2019/058255 and WO 2019/058253 describe methods for silencing gene expression in eukaryotic cells that utilise a DNA editing agent and involve modification of an endogenous locus in situ and in vivo.

Improved methods for silencing expression of target genes are required.

### SUMMARY OF THE INVENTION

The inventors have developed an improved system for silencing the expression of target genes in eukaryotes. The system of the invention is efficient, utilises minimal genetic modifications, and achieves a range of silencing effects. In particular, in the system of the invention an endogenous silencing sequence is identified that has a desired expression pattern and that can be modified and redirected to silence the target gene with minimal nucleotide changes. The endogenous silencing sequence, including its promoter and terminator, is used to generate a silencing insertion that includes minimal nucleotide changes in order to provide specificity for the target gene. The silencing insertion is inserted into the genome of the eukaryotic cell to provide specific silencing of the target gene in desired tissues and conditions.

The silencing insertion is generated from an endogenous silencing sequence, including its promoter and terminator, and so it is highly similar to the endogenous sequence. Therefore, advantageously, the silencing insertion is processed efficiently by the host because the sequences of the insertion and their arrangement are endogenous to the host. Also, the silencing insertion is a cisgenic modification that introduces endogenous sequence in its endogenous arrangement with no juxtaposition of promoters and silencing constructs, which is attractive to consumers and presents greatly reduced potential issues for regulators.

The silencing insertion is inserted randomly into the genome, which is highly efficient. Inclusion of a promoter and terminator in the silencing insertion allows it to be expressed effectively when inserted randomly and can avoid the need for targeting sequences. Insertion of a sequence also allows for selection of successful transformants, if the insertion comprises a selectable marker.

The silencing insertion of the invention benefits from introducing an endogenous sequence in its endogenous arrangement with minimal nucleotide changes and also benefits from specific silencing of target genes in desired tissues and conditions. This is achieved, in part, because the insertion maintains the link between the endogenous sequence encoding a silencing RNA and its cognate promoter and terminator, and also other sequences that control expression such as 5′UTR and 3′UTR sequences, whilst also including modification to the targeting sequence in order to redirect silencing to the target gene of interest.

Accordingly, the invention provides a method of reducing the expression of a first target gene comprising inserting a silencing insertion into the genome of a eukaryotic cell,
wherein the silencing insertion comprises a promoter or a part thereof, a sequence encoding a silencing RNA that silences the expression of the first target gene, and a terminator or a part thereof,
wherein the silencing insertion has over 95% sequence identity across its length to an endogenous silencing sequence in the eukaryotic cell that comprises a promoter, a sequence encoding a silencing RNA and a terminator, and
wherein the silencing RNA encoded by the endogenous silencing sequence is not active or silences the expression of a second target gene that is different from the first target gene.

The invention also provides a eukaryotic cell comprising in its genome:
(a) a silencing insertion comprising a promoter or a part thereof, a sequence encoding a silencing RNA that silences the expression of a first target gene, and a terminator or a part thereof,
(b) an endogenous genome sequence that has been separated by the insertion, and
(c) an endogenous silencing sequence comprising a promoter, a sequence encoding a silencing RNA and a terminator,
wherein the silencing insertion and the endogenous silencing sequence have over 95% sequence identity across the length of the insertion, and
wherein the silencing RNA encoded by the endogenous silencing sequence is not active or silences the expression of a second target gene that is different from the first target gene.

The invention also provides an isolated silencing construct comprising a silencing insertion that comprises a promoter or a part thereof, a sequence encoding a silencing RNA that silences the expression of a first target gene in a eukaryotic cell, and a terminator or a part thereof,
wherein the silencing insertion has over 95% sequence identity across its length to an endogenous silencing sequence in the eukaryotic cell that comprises a promoter, a sequence encoding a silencing RNA and a terminator, and
wherein the silencing RNA encoded by the endogenous silencing sequence is not active or silences the expression of a second target gene that is different from the first target gene.

In preferred embodiments, the silencing insertion is inserted by particle bombardment (biolistic bombardment), protoplast transfection, electroporation or nanoparticle-mediated transfection. Such techniques do not require transgenic DNA, and so they introduce only the minimal genetic changes that are provided by the system of the invention.

In preferred embodiments, the eukaryotic cell is a plant cell. Methods of silencing target genes whilst making minimal genetic changes are particularly useful in plants because they allow the generation of crops and plant produce that do not comprise exogenous DNA or endogenous DNA sequences that are not in their endogenous arrangement, which is preferred by consumers and regulators. It is also possible to breed out transgenic DNA in plants.

In preferred embodiments, the eukaryotic cell is a banana plant cell or a coffee plant cell or a rice plant cell. The methods and products of the invention are particularly useful for silencing genes in banana plants, because they are asexual and it is impossible to breed bananas to achieve improved traits or phenotypes. Similarly, the methods and products of the invention are particularly useful for silencing genes in coffee plants, because breeding coffee plants takes 15-25 years and is uneconomical. Similarly, the methods and products of the invention are particularly useful for silencing genes in rice plants, because breeding rice plants is particularly time consuming.

In alternative embodiments, the eukaryotic cell is an animal cell, preferably a livestock animal cell. A livestock animal may be any breed or population of animal kept by humans for a useful, commercial purpose. Examples of livestock animals for use in the invention include horse, donkey, cattle or any other ruminants, zebu, yak, buffalo, sheep, goat, reindeer, camel, llama, alpaca, pig, rabbit or any other rodent, dog, cat, poultry such as chicken, turkey, goose or duck, fish, crustaceans and molluscs. A livestock animal is not a human. Methods of silencing target genes whilst making minimal genetic changes are particularly useful in livestock animals because they allow the generation of animals and animal produce that do not comprise exogenous DNA or endogenous DNA sequences that are not in their endogenous arrangement, which is preferred by consumers and regulators. It is also possible to breed out transgenic DNA in livestock animals. The invention also provides methods of treating or preventing disease in livestock animals comprising administering a silencing insertion or animal cell of the invention, and provides silencing insertions and animal cells of the invention for use in treating or preventing disease.

In alternative embodiments, the eukaryotic cell is a human cell. Methods of silencing target genes whilst making minimal genetic changes are particularly useful in humans because they allow therapeutic sequences to be introduced with minimal other changes that might be deleterious. The invention also provides methods of treating or preventing disease in humans comprising administering a silencing insertion or human cell of the invention, and provides silencing insertions and human cells of the invention for use in treating or preventing disease. In embodiments using human cells, the cells are isolated and handled in vitro, or the methods are therapeutic. According to some embodiments, the eukaryotic cell is a human cell isolated from the human body, wherein the silencing insertion of the invention is introduced in vitro, optionally wherein the human cell with the silencing insertion of the invention is re-introduced into a human. According to some embodiments, the invention provides methods of treating or preventing disease in humans comprising: (1) isolating a cell from a human body; (2) introducing the silencing insertion of the invention into the cell in vitro, resulting in a modified cell; and (3) administering the modified cell into a human subject, optionally the same human subject from which the non-modified cell originated. According to some embodiments, in such a method the silencing insertion of the invention introduces silencing specificity towards a target gene associated with the disease that the method is treating or preventing.

In preferred embodiments, the silencing insertion comprises 1-40, such as 5-40, 5-30, 5-20, 3-40, 3-30, 3-20, 5-15, 3-10, 10-30, or 10-20, nucleotide additions, deletions and substitutions relative to the endogenous silencing sequence. Such alterations are suitable for redirecting the endogenous silencing sequence to target the target gene of interest, whilst presenting only minimal changes to the endogenous sequence. Therefore, preferably these changes are in the sequence encoding the silencing RNA, and preferably the promoter and terminator, and any other sequences that control expression such as 5′UTR and 3′UTR sequences, in the silencing insertion are identical to the corresponding sequences in the endogenous silencing sequence.

In preferred embodiments, the silencing insertion is adjacent to a selectable marker and the method of the invention comprises inserting the silencing insertion with the adjacent selectable marker into the genome of a eukaryotic cell. Use of a selectable marker allows the selection of successful transformants, which makes their generation much more efficient. Preferably, the selectable marker is highly similar to an endogenous gene of the eukaryotic cell, such as a mutant version of a gene endogenous to the eukaryotic cell, to minimise the exogenous genetic material that is introduced. The selectable marker can be a mutant version of an endogenous gene of the eukaryotic cell, optionally a gene which does not function as a marker until the mutation (preferably a dominant mutation) is introduced and enables it to function as a selectable marker. A selectable marker which is a mutant version of an endogenous gene is preferably used with the promotor and terminator of the endogenous gene. Preferably the mutant version of the marker includes a mutation which naturally exists in another eukaryotic cell of the same genus or the same species. A non-limiting example of a selectable marker is a mutated version of an ALS gene that confers chlorsulfuron herbicide resistance. The system of the invention allows multiple silencing insertions to be inserted together, providing silencing of multiple genes and “stacking” of traits. This is particularly advantageous for use with asexual crops, such as banana, which cannot be crossed to combine traits.

The invention also provides plants, plant parts, seeds, fruit and plant products obtained from the methods of the invention and comprising cells with silencing insertions of the invention. Such products will exhibit the improved traits provided by specific gene silencing, whilst being cisgenic and not containing any exogenous DNA or endogenous DNA in a non-endogenous arrangement.

The invention also provides animals and animal products obtained from the methods of the invention and comprising cells with silencing insertions of the invention. Such animals and products will exhibit the improved traits provided by specific gene silencing, whilst being cisgenic and not containing any transgenic DNA or endogenous DNA in a non-endogenous arrangement.

### BRIEF DESCRIPTION OF THE FIGURES

FIG. 1—Promoter identification pipeline: Process Overview.

FIG. 2—Terminator identification pipeline: Process Overview.

FIG. 3. WT (A) and GEiGS™-Insertion (B) sequences and stem-loop structures (C) for a miRNA example (ath-miR173). Promoter, ncRNA (non-coding RNA) and terminator regions are indicated in underlined, italics and italics+underlined, respectively. Changes from the WT sequence are indicated in bold. 5′ to 3′ orientation is indicated.

FIG. 4. WT (A) and GEiGS™-Insertion (B) sequences for a tasiRNA example (ath-Tas3a). Promoter, ncRNA (non-coding RNA) and terminator regions are indicated in underlined, italics and italics+underlined, respectively. Changes from the WT sequence are indicated in bold. 5′ to 3′ orientation is indicated.

FIG. 5. Overlapping PCR strategy to introduce GEiGS™-Insertion modifications. Two separate PCR reactions with primers A+B (PCR 1) and C+D (PCR 2), respectively, are carried out using genomic DNA as template. Primers B and C contain the modified GEiGS™ sequence at their 5′(dotted lines). A third PCR reaction (PCR 3) is carried out annealing the modified region of products from PCR 1 and 2 and using primers A+D allowing to amplify the whole GEiGS™-Insertion cassette.

FIG. 6. GEiGS™-Insertion with selection diagrams. The GEiGS™-Insertion cassette can be designed in such a way that the GEiGS™ ncRNA and mutated P197S expression cassettes face the opposite directions (A) or the same direction (B). Orientation A is preferred to prevent read-through transcription. ALS cassette is natively expressed from the complementary strand. ALS endogenous promoter (including 5′UTR) and terminator (including 3′UTR) regions are shown. ncRNA endogenous promoter and terminator are shown. GEiGS™ ncRNA contains minimum mutations, which do not affect its secondary structure, to redirect the silencing activity and specificity of the sRNA from that of the silencing gene on which the insertion is based. ALS P197S: single point ALS mutant in which proline 197 was mutated to serine to confer chlorsulfuron resistance. cTP: chloroplast transit peptide, which is part of the ALS open reading frame.

FIG. 7. GEiGS™-Insertion with selection. ALS/ALS P197S endogenous promoter (including 5′UTR) and terminator (including 3′UTR) regions are shown. miR173 endogenous promoter and terminator are shown. Minimum mutations in miR173 GEiGS™ sequence are marked as spheres. These mutations do not affect the ncRNA secondary structure and allow to redirect the silencing activity and specificity of the sRNA. ALS P197S: single point ALS mutant in which proline 197 was mutated to serine to confer chlorsulfuron resistance. cTP: chloroplast transit peptide, which is part of the ALS open reading frame. ALS/ALS P197S and miR173 are natively expressed from the complementary and sense strand, respectively.

FIG. 8. GEiGS™ combo-Generation of transgenesis-based and HDR-based GEiGS™ plant lines in one T-DNA segment. The GEiGS™ combo approach facilitates the generation of HDR-based GEiGS™ plant lines (left) and cis-genic-based GEiGS™ lines (right) using the same T-DNA segment. This segment contains expression cassettes to carry out DNA break (e.g CRISPR/CAS9 and sgRNA), a DNA donor template (to facilitate the introduction of the modified sequence through HDR pathway) and an option for a selection or reporter marker, to enrich for transformation events. (A) The donor sequence serves as a donor template, that is used by the cell, via the HDR pathway, following the DNA break generated by the CRISPR/CAS and sgRNA cassettes. This donor template introduces the desired DNA modification, in the endogenous scaffold locus, to introduce the sequence of the GEiGS™ solution. This will result in the GEiGS™ solution gene to be expressed from its native location in the genome. This function of the GEiGS™ combo method can be executed in a non-GM manner, where the gene editing event is carried out through transient expression of the T-DNA cassettes. In addition, this process could be carried out in a GM manner (i.e T-DNA integration in the plant genome), resulting in a GM plant, and the T-DNA can be breed out in the following generations. (B) In order to execute the GEiGS™-mimic approach in the GEiGS™ combo method, the donor region is designed in a way that it must contain, at least, the GEiGS™ solution sequence, with the native promoter and terminator of the scaffold, that the solution is based on. If a longer sequence is required, for the HDR-based method, this can be further extended, and is represented as “genomic sequence” in the figure, to generate longer homologous arms. Once the T-DNA is incorporated in the plant genome, this region will be active as the endogenous scaffold, and the GEiGS™ solution will be expressed and processed. This will result in a relevant required trait via GEiGS™ mimic, in a GM manner. In other words, this method introduces GEiGS™ through a cis-genic approach, by expression from integrated T-DNA cassette.

FIG. 9. Example of a GEiGS™-Insertion cassette. GEiGS™-Insertion cassettes (here referred as “DONOR”) are 1.2 kb in length, spanning the modified region required to redirect the silencing specificity of the miRNA (here referred as “GEiGS solution”) plus approximately 500 bp upstream and downstream of this site.

FIG. 10. HDR genotype of GEiGS™-Insertion plants used for sRNA-seq. A small piece of leaf tissue was harvested from each plant and direct PCR was performed using a forward primer specific to the intended HDR edits and a reverse primer external to the HDR donor/GEiGS™-Insertion cassette, i.e. specific to the native scaffold. As a positive control, a plasmid containing both the HDR donor/GEiGS™-Insertion sequence and the flanking region sequences was used. PCR reactions were loaded in a 0.8% agarose gel stained with ethidium bromide and visualised under UV light.

FIG. 11. HDR genotype of GEiGS™-Insertion plants used for TuMV bioassay. A small piece of leaf tissue was harvested from each plant and direct PCR was performed using a forward primer specific to the intended HDR edits and a reverse primer external to the HDR donor/GEiGS™-Insertion cassette, i.e. specific to the native scaffold. As a positive control, a plasmid containing both the HDR donor/GEiGS™-Insertion sequence and flanking region sequences was used. PCR reactions were loaded in a 0.8% agarose gel stained with ethidium bromide and visualised under UV light.

FIG. 12. GFP area analysis. (A) TuMV:GFP-infected A. thaliana plants were photographed with a digital camera mounted on a tripod with fixed distance and settings under UV illumination to investigate GFP spreading. (B) A custom image analysis pipeline was developed in CellProfiler 3.1.9 to determine the proportion of total leaf area occupied by GFP fluorescence. An output image overlaying the GFP area was saved for quality-control. (C) Images saved for quality control were reviewed manually and those with large errors had the total leaf area and TuMV area measured manually using ImageJ.

FIG. 13. Percentage of total leaf area occupied by GFP fluorescence. TuMV:GFP-infected A. thaliana plants were photographed under UV illumination 7, 11, 15, and 17 days post inoculation (dpi). Total leaf area and TuMV:GFP area were measured using a custom image analysis pipeline developed in CellProfiler 3.1.9 and manually curated using ImageJ. The percentage of TuMV:GFP area over total leaf area was calculated for individual plants at each timepoint. Box plots show the median (central band) and interquartile range (IQR, boxes)±minimum and maximum values (whiskers). Outliers are shown as small circles. n=5 plants per group.

### DETAILED DESCRIPTION OF THE INVENTION

### Silencing Insertion Design

The invention provides methods of reducing the expression of target genes comprising inserting a silencing insertion, provides eukaryotic cells carrying the silencing insertions, and provides the silencing insertions themselves. The silencing insertions of the invention provide tailored, flexible and specific silencing effects whilst introducing only endogenous DNA in its endogenous arrangement.

The silencing insertions of the invention utilise an endogenous sequence encoding a silencing RNA in combination with its cognate promoter and terminator, and also other sequences that control expression such as 5′UTR and 3′UTR sequences. In order to design the silencing insertions of the invention minimal sequence modifications are made to the sequence encoding the silencing RNA in order to redirect silencing to the target gene of interest (and possibly maintain the secondary structure of the silencing RNA encoded by the silencing insertion, when essential to its function).

Design of the silencing insertions of the invention requires identifying a suitable endogenous starting sequence. The starting sequence must comprise a promoter and terminator and optionally other sequences that control expression such as 5′UTR and 3′UTR sequences that provide the desired expression pattern, for example, in terms of strength of expression, timing of expression and/or tissue of expression. The starting sequence must also comprise a sequence encoding a silencing RNA that can be redirected to the target gene of interest with minimal nucleotide changes. According to some embodiments, the starting sequence can comprise a sequence encoding a non-coding RNA which is similar to a silencing RNA sequence in the eukaryotic cell but not processed as one. In such embodiments, the modifications introduced into that sequence in the context of the silencing insertion reactivate the silencing capability of the encoded RNA and redirect its silencing specificity towards the target sequence of choice. Identification of such starting sequences and the process of identifying modifications necessary for reactivation of silencing activity can be done essentially as described in WO 2020/183419. Examples 1 and 2 provide exemplary processes and useful databases that can be used to identify appropriate starting sequences (referred to as non-coding RNA, ncRNA).

Accordingly, starting sequences are identified through bioinformatics searching and analysis that analyses both the target sequence of an endogenous silencing sequence, but also its promoter and terminator sequences and expression profile. The searching and analysis may also consider G/C content, length of promoter, silencing sequence and/or terminator, and secondary structure. In preferred embodiments, the identification of the starting endogenous silencing sequence and/or the design of the silencing insertion are performed using an automated computational platform. The automated computational platform may provide multiple options for targeting a particular gene, for example with different degrees of editing required, different expression profiles of the silencing insertion, and different potency.

In order to silence the target gene with minimal nucleotide alterations, the target gene silenced by the endogenous silencing sequence may exhibit sequence identity to the target gene that is to be silenced by the silencing insertion, such as at least 60, 70, 80, 85, 90 or 95% sequence identity. The identity will generally not be 100% because the silencing insertion and the endogenous silencing sequence target different genes.

In certain embodiments, the sequence identity of the different parts of the silencing insertion to their corresponding sequences in the endogenous sequence may be defined independently. In certain such embodiments, the promoter in the silencing insertion and the promoter in the endogenous silencing sequence have at least 90%, such as at least 92%, 94%, 96%, 98%, 99%, 99.5% or 99.9% sequence identity across the length of the promoter. In certain such embodiments, the terminator in the silencing insertion and the terminator in the endogenous silencing sequence have at least 90%, such as at least 92%, 94%, 96%, 98%, 99%, 99.5% or 99.9% sequence identity across the length of the terminator. In certain such embodiments, the sequence encoding a silencing RNA in the silencing insertion and the sequence encoding a silencing RNA in the endogenous silencing sequence have at least 60%, such as at least 70%, 80%, 85%, 90%, 92%, 94%, 96%, 98%, 99%, 99.2%, 99.4%, 99.6%, or 99.9% sequence identity across the length of the sequence encoding the silencing RNA. In certain such embodiments, the promoter in the silencing insertion and the promoter in the endogenous silencing sequence are essentially identical (other than single nucleotide polymorphisms), the terminator in the silencing insertion and the terminator in the endogenous silencing sequence are essentially identical (other than single nucleotide polymorphisms), and the sequence encoding a silencing RNA in the silencing insertion and the sequence encoding a silencing RNA in the endogenous silencing sequence have at least 60%, such as at least 70%, 80%, 85%, 90%, 92%, 94%, 96%, 98%, 99%, 99.2%, 99.4%, 99.6%, or 99.9% sequence identity across the length of the sequence encoding the silencing RNA. In such embodiments, the promoter in the endogenous silencing sequence, the terminator in the endogenous silencing sequence and the sequence encoding a silencing RNA in the endogenous silencing sequence are all present in the same endogenous silencing sequence.

In certain embodiments, the sequence identity of the different parts of the silencing insertion to their corresponding sequences in the endogenous sequence may be defined independently and with different measures. In certain such embodiments, the promoter in the silencing insertion and the promoter in the endogenous silencing sequence have at least 90%, such as at least 92%, 94%, 96%, 98%, 99%, 99.5% or 99.9% sequence identity across the length of the promoter. In certain such embodiments, the terminator in the silencing insertion and the terminator in the endogenous silencing sequence have at least 90%, such as at least 92%, 94%, 96%, 98%, 99%, 99.5% or 99.9% sequence identity across the length of the terminator. In certain such embodiments, wherein the sequence encoding a silencing RNA in the silencing insertion comprises 1-40, such as 5-40, 5-30, 5-20, 3-40, 3-30, 3-20, 5-15, 3-10, 10-30, or 10-20 nucleotide additions, deletions and substitutions relative to the sequence encoding a silencing RNA in the endogenous silencing sequence. In such embodiments, the promoter in the endogenous silencing sequence, the terminator in the endogenous silencing sequence and the sequence encoding a silencing RNA in the endogenous silencing sequence are all present in the same endogenous silencing sequence.

In any of the embodiments described herein, references to the promoter and the terminator in the silencing insertion may include any other sequences present at the endogenous silencing locus that control expression such as 5′UTR and 3′UTR sequences.

In certain embodiments, the silencing insertion comprises an additional nucleotide sequence in addition to the promoter or a part thereof, the sequence encoding a silencing RNA, the terminator or a part thereof and other sequences from the locus that control expression such as 5′UTR and 3′UTR sequences. In particular, when designing the silencing insertion it is not necessary to precisely limit the 5′ and 3′ ends to the endogenous promoter and terminator. It may be that the exact limits of the endogenous promoter and terminator sequences are not known. Any additional sequence in the silencing insertion will still be derived from the same endogenous silencing sequence (upstream to the promotor sequence and/or downstream to the terminator sequence) and so will have high identity to the endogenous silencing sequence. Thus, in certain embodiments, the silencing insertion consists of a promoter or a part thereof, a sequence encoding a silencing RNA, a terminator or a part thereof, and additional sequence adjacent to the promoter and/or terminator, wherein the silencing insertion including the additional sequence has over 95% sequence identity across its length to the endogenous silencing sequence and corresponding promoter, sequence encoding a silencing RNA, terminator and additional sequence adjacent to the promoter and/or terminator. In certain such embodiments, the silencing insertion comprises the sequence encoding a silencing RNA and at least 50, 100, 250, 500, 750, 1000, 2000 or 5000 base pairs 5′ of the silencing RNA and at least 20, 50, 100, 250, 500, 750, 1000 or 2000 base pairs 3′ of the silencing RNA, which will generally include a promoter and a terminator. In certain such embodiments, the silencing insertion comprises the sequence encoding a silencing RNA and 50-5000, 100-2500, 200-1500, or 400-1000 base pairs 5′ of the silencing RNA and 20-2000, 50-1000, 100-750 or 150-500 base pairs 3′ of the silencing RNA, which will generally include a promoter and a terminator.

In alternative embodiments, the 5′ end of the silencing insertion is contiguous with the 5′ end of the promoter and/or the 3′ end is contiguous with the 3′ of the terminator.

In certain embodiments, the silencing insertion is adjacent to an additional sequence that is not highly identical to sequence at or adjacent to the endogenous silencing sequence. In such methods of the invention the silencing insertion is inserted into into the genome of the eukaryotic cell with the adjacent additional sequence. For example, the silencing insertion may be linked to a selectable marker, as discussed further below. In such embodiments, the sequence identity between the silencing insertion and the endogenous sequence is calculated by excluding the adjacent additional sequence. In certain such embodiments, the adjacent additional sequence has at least 90%, such as at least 92%, 94%, 96%, 98%, 99%, 99.5% or 99.9% sequence identity to a different endogenous sequence. For example, in particular embodiments, the silencing insertion is adjacent to a polynucleotide sequence, wherein the polynucleotide sequence comprises a promoter or a part thereof, a sequence encoding a selectable marker, and a terminator or a part thereof, wherein the polynucleotide sequence has at least 90%, such as at least 92%, 94%, 96%, 98%, 99%, 99.5% or 99.9% sequence identity across its length to an endogenous sequence in the eukaryotic cell that comprises a promoter, a sequence encoding an endogenous gene and a terminator. In certain embodiments, the sequence encoding the endogenous gene does not function as a selectable marker.

In alternative embodiments, the methods of the invention comprise insertion of the silencing insertion and no adjacent additional sequence. The method therefore comprises inserting a construct that consists of the silencing insertion. In such embodiments, the eukaryotic cells of the invention comprise the silencing insertion and the endogenous genome sequence that has been separated by the insertion is present at the 5′ and 3′ ends of the insertion.

According to some embodiments of the invention, the silencing insertion is inserted randomly in the genome of the eukaryotic cell. Random insertion does not require additional exogenous targeting sequence and is more efficient. Accordingly, in certain embodiments, the silencing insertion does not include targeting sequence and is not present in a vector or composition that includes targeting sequence. As a result of the random insertion of the silencing insertion, eukaryotic cells of the invention comprise an endogenous genome sequence that has been separated by the insertion. The sequence adjacent to either end of the silencing insertion (and any additional adjacent sequence such as a selectable marker) will be further apart than in the absence of the insert. Some small amount of endogenous sequence may be deleted in the random insertion, in between the sequence that is now separated by the silencing insertion (and any additional adjacent sequence such as a selectable marker). The eukaryotic cells of the invention are therefore distinguished from cells modified with genome editing technology that utilizes homologous recombination or homology directed repair, for example, because such cells do not comprise endogenous genome sequence that has been separated by an insertion. Similarly, the methods of the invention comprise inserting a silencing insertion and this inserting, consistent with the normal meaning of the term, does not comprise replacement of any homologous sequence.

In certain embodiments, the location of the silencing insertion can be defined relative to the endogenous silencing insertion, rather than in terms of the endogenous sequence that is separated by the insertion. For example, the silencing insertion may be present at a location in the genome that does not encode a silencing RNA in the absence of the insertion, or the silencing insertion and the endogenous silencing sequence may be present on different chromosomes or at different locations on the same chromosome separated by at least 5 kb, 10 kb, 50 kb or 100 kb.

In certain embodiments, the silencing insertions of the invention may be 1-10 kb, such as 2-5 kb.

### Silencing RNA

The invention provides silencing insertions that target specific genes of interest using endogenous sequence in its endogenous arrangement with minimal nucleotide changes that alter the targeting of an endogenous silencing RNA.

The silencing RNA encoded by the silencing insertion may be, or may be processed into, a small interfering RNA (siRNA), a microRNA (miRNA), a piwi-interacting RNA (piRNA), a phased small interfering RNA (phasiRNA), a trans-acting siRNA (tasiRNA), a short-hairpin RNA (shRNA), inverted repeat RNA forming double stranded RNA, a small nuclear RNA (snRNA or U-RNA), a small nucleolar RNA (snoRNA), a Small Cajal body RNA (scaRNA), a transfer RNA (IRNA), a ribosomal RNA (rRNA), a repeat-derived RNA, an autonomous or non-autonomous transposable or retro-transposable element-derived RNA, an autonomous or non-autonomous transposable or retro-transposable element RNA, a transfer RNA fragment (tRF), or a long non-coding RNA (lncRNA). In preferred embodiments, silencing RNA is a small interfering RNA (siRNA) or a microRNA (miRNA). In further preferred embodiments, the silencing RNA is a small interfering RNA (siRNA), short hairpin RNA (shRNA), microRNA (miRNA), Piwi-interacting RNA (piRNA), phased small interfering RNA (phasiRNA) or a trans-acting siRNA (tasiRNA).

In preferred embodiments, the silencing RNA encoded by the silencing insertion and the silencing RNA encoded by the endogenous silencing sequence are the same type of silencing RNA, such as a small interfering RNA (siRNA), a microRNA (miRNA), a piwi-interacting RNA (piRNA), a phased small interfering RNA (phasiRNA), a trans-acting siRNA (tasiRNA), a short-hairpin RNA (shRNA), inverted repeat RNA forming double stranded RNA, a small nuclear RNA (snRNA or U-RNA), a small nucleolar RNA (snoRNA), a Small Cajal body RNA (scaRNA), a transfer RNA (tRNA), a ribosomal RNA (rRNA), a repeat-derived RNA, an autonomous or non-autonomous transposable or retro-transposable element-derived RNA, an autonomous or non-autonomous transposable or retro-transposable element RNA, a transfer RNA fragment (tRF), or a long non-coding RNA (lncRNA). In preferred embodiments, silencing RNA is a small interfering RNA (siRNA) or a microRNA (miRNA).

In certain embodiments, the silencing RNA molecule is an RNA interference (RNAi) molecule.

In order to redirect the endogenous silencing sequence, nucleotide alterations are introduced into the silencing insertion. The targeting mechanisms of various different types of silencing RNA molecules are well understood and the targeting of silencing RNA molecules can be modelled in silico, which allows appropriate nucleotide alterations to be selected in design of the silencing insertions of the invention. The alterations may be nucleotide substitutions, nucleotide deletions and/or nucleotide insertions.

The specific binding of the silencing RNA with the target gene sequence can be determined by computational algorithms (such as BLAST) and verified by methods including e.g. Northern blot, In Situ hybridization, QuantiGene Plex Assay etc.

In certain embodiments, the alterations are in a stem region of the silencing RNA. In certain embodiments, the alterations are in a loop region of the silencing RNA. In certain embodiments, the alterations are in a non-structured region of the silencing RNA. In certain embodiments, the alterations are in a stem region and a loop region of the silencing RNA. In certain embodiments, the alterations are in a stem region and a loop region and a non-structured region of the silencing RNA.

In certain embodiments, the sequence encoding a silencing RNA in the silencing insertion comprises 1-40, such as 5-40, 5-30, 5-20, 3-40, 3-30, 3-20, 5-15, 10-30, 10-20, 3-10 or 5-10, nucleotide additions, deletions and/or substitutions relative to the sequence encoding a silencing RNA in the endogenous silencing sequence. In preferred embodiments, the nucleotide alterations are not contiguous, or do not comprise more than 10, more than 8, more than 6, more than 4, or more than 2 contiguous alterations. In preferred embodiments, the nucleotide alterations are all substitutions.

According to some embodiments, the silencing activity of the silencing RNA encoded by the endogenous silencing sequence is affected by the secondary structure of the silencing RNA (such as, but not limited to, in the case of a silencing RNA encoding miRNA). In preferred embodiments, where the silencing activity of the silencing RNA encoded by the endogenous silencing sequence is affected by the secondary structure of the silencing RNA, the silencing RNA encoded by the silencing insertion and the silencing RNA encoded by the endogenous silencing sequence form the same secondary structure. In such embodiments, when the alterations are made to the silencing RNA to redirect its targeting, modeling may be used to ensure that the alterations do not ablate secondary structures. Maintaining the secondary structure may help ensure the RNA encoded by the silencing insertion is appropriately recognized and processed by the cellular silencing machinery. Secondary structure can be maintained by maintaining the base pairing profile.

In order to silence the target gene, the silencing RNA encoded by the silencing insertion will generally comprise sequence that is complementary to the target gene. This means the silencing RNA molecule (or at least a portion of it that is present in the processed silencing RNA, or at least one strand of a double-stranded polynucleotide or portion thereof, or a portion of a single strand polynucleotide) hybridizes under physiological conditions to the target RNA, or a fragment thereof, to effect regulation or function or suppression of the target gene. For example, in some embodiments, a silencing RNA molecule has 100 percent sequence identity or at least 80, 85, 90, 91, 92, 93, 94, 95, 96, 97, 98, or 99 percent sequence identity when compared to a sequence of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 70, 80, 90, 100, 150, 200, 300, 400, 500 or more contiguous nucleotides in the target RNA. If the silencing RNA is a miRNA, the complementarity may be over the seed sequence or over the sequence of the mature miRNA.

Preferably, the silencing RNA will comprise sequence that exhibits complete complementarity to the target gene, such that every nucleotide of one of the sequences read 5′ to 3′ is complementary to every nucleotide of the other sequence when read 3′ to 5′. A nucleotide sequence that is completely complementary to a reference nucleotide sequence will exhibit a sequence identical to the reverse complement sequence of the reference nucleotide sequence. Methods for determining sequence complementarity are well known in the art and include, but not limited to, bioinformatics tools which are well known in the art (e.g. BLAST, multiple sequence alignment).

The term “RNA silencing” or RNAi refers to a cellular regulatory mechanism in which non-coding RNA molecules (the “RNA silencing molecule” or “RNAi molecule”) mediate, in a sequence specific manner, co- or post-transcriptional inhibition of gene expression or translation. In some embodiments, the silencing RNA is capable of mediating RNA repression during transcription (co-transcriptional gene silencing). In some embodiments, co-transcriptional gene silencing includes epigenetic silencing (e.g. chromatic state that prevents gene expression). In some embodiments, the silencing RNA is capable of mediating RNA repression after transcription (post-transcriptional gene silencing). Post-transcriptional gene silencing typically refers to the process of degradation or cleavage of messenger RNA (mRNA) molecules which decrease their activity by preventing translation. For example, a guide strand of an RNA silencing molecule pairs with a complementary sequence in a mRNA molecule and induces cleavage by e.g. Argonaute 2 (Ago2).

Co-transcriptional gene silencing typically refers to inactivation of gene activity (i.e. transcription repression) and typically occurs in the cell nucleus. Such gene activity repression is mediated by epigenetic-related factors, such as e.g. methyl-transferases, that methylate target DNA and histones. Thus, in co-transcriptional gene silencing, the association of a small RNA with a target RNA (small RNA-transcript interaction) destabilizes the target nascent transcript and recruits DNA- and histone-modifying enzymes (i.e. epigenetic factors) that induce chromatin remodelling into a structure that repress gene activity and transcription. Also, in co-transcriptional gene silencing, chromatin-associated long non-coding RNA scaffolds may recruit chromatin-modifying complexes independently of small RNAs. These co-transcriptional silencing mechanisms form RNA surveillance systems that detect and silence inappropriate transcription events, and provide a memory of these events via self-reinforcing epigenetic loops [as described in D. Hoch and D. Moazed, RNA-mediated epigenetic regulation of gene expression, Nat Rev Genet. (2015) 16(2): 71-84].

In some embodiments, the silencing RNA is processed by the RNAi biogenesis/processing machinery. In certain embodiments, the silencing RNA is a capable of inducing RNA interference (RNAi). In certain embodiments, the silencing RNA expressed by the silencing insertion is a precursor that is processed into a smaller silencing RNA molecule. In certain embodiments, the silencing RNA is a single stranded RNA (ssRNA) precursor. In certain embodiments, the silencing RNA is a duplex-structured single-stranded RNA precursor. In certain embodiments, the silencing RNA is a dsRNA precursor (e.g. comprising perfect and imperfect base pairing).

Perfect and imperfect based paired RNA (i.e. double stranded RNA; dsRNA), siRNA and shRNA—The presence of long dsRNAs in cells stimulates the activity of a ribonuclease III enzyme referred to as dicer. Dicer (also known as endoribonuclease Dicer or helicase with RNase motif) is an enzyme that in plants is typically referred to as Dicer-like (DCL) protein. Different plants have different numbers of DCL genes, thus for example, Arabidopsis genome typically has four DCL genes, rice has eight DCL genes, and maize genome has five DCL genes. Dicer is involved in the processing of the dsRNA into short pieces of dsRNA known as short interfering RNAs (siRNAs). siRNAs derived from dicer activity are typically about 21 to about 24 nucleotides in length and comprise about 19 base pair duplexes with two 3′ nucleotides overhangs.

Accordingly, in some embodiments of the invention an endogenous gene encoding a dsRNA is isolated, modified and used to prepare a silencing insertion to redirect silencing activity towards a new gene.

In certain embodiments, a dsRNA precursor longer than 21 bp is used. Various studies demonstrate that long dsRNAs can be used to silence gene expression without inducing the stress response or causing significant off-target effects-see for example [Strat et al., Nucleic Acids Research, 2006, Vol. 34, No. 13 3803-3810; Bhargava A et al. Brain Res. Protoc. 2004; 13:115-125; Diallo M., et al., Oligonucleotides. 2003; 13:381-392; Paddison P. J., et al., Proc. Natl Acad. Sci. USA. 2002; 99:1443-1448; Tran N., et al., FEBS Lett. 2004; 573:127-134].

The term “siRNA” refers to small inhibitory RNA duplexes (generally between 18-30 base pairs) that induce the RNA interference (RNAi) pathway. Typically, siRNAs are chemically synthesized as 21 mers with a central 19 bp duplex region and symmetric 2-base 3′-overhangs on the termini, although it has been recently described that chemically synthesized RNA duplexes of 25-30 base length can have as much as a 100-fold increase in potency compared with 21 mers at the same location. The observed increased potency obtained using longer RNAs in triggering RNAi is suggested to result from providing Dicer with a substrate (27 mer) instead of a product (21 mer) and that this improves the rate or efficiency of entry of the siRNA duplex into RISC.

It has been found that position, but not the composition, of the 3′-overhang influences potency of a siRNA and asymmetric duplexes having a 3′-overhang on the antisense strand are generally more potent than those with the 3′-overhang on the sense strand (Rose et al., 2005).

The strands of a double-stranded interfering RNA (e.g., a siRNA) may be connected to form a hairpin or stem-loop structure (e.g., a shRNA). Thus, as mentioned, the silencing RNA of some embodiments of the invention may also be a short hairpin RNA (shRNA).

The term short hairpin RNA, “shRNA”, as used herein, refers to an RNA molecule having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogues) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop are provided in WO2013126963 and WO2014107763. It will be recognized by one of skill in the art that the resulting single chain oligonucleotide forms a stem-loop or hairpin structure comprising a double-stranded region capable of interacting with the RNAi machinery.

Various types of siRNAs are contemplated by the present invention, including trans-acting siRNAs (Ta-siRNAs or TasiRNA), repeat-associated siRNAs (Ra-siRNAs) and natural-antisense transcript-derived siRNAs (Nat-siRNAs).

According to one embodiment, silencing RNA includes “piRNA” which is a class of Piwi-interacting RNAs of about 26 and 31 nucleotides in length. piRNAs typically form RNA-protein complexes through interactions with Piwi proteins, i.e. antisense piRNAs are typically loaded into Piwi proteins (e.g. Piwi, Ago3 and Aubergine (Aub)).

miRNA—According to another embodiment the silencing RNA may be a miRNA.

The term “microRNA”, “miRNA”, and “miR” are synonymous and refer to a collection of non-coding single-stranded RNA molecules of about 19-24 nucleotides in length, which regulate gene expression. miRNAs are found in a wide range of organisms (e.g. insects, mammals, plants, nematodes) and have been shown to play a role in development, homeostasis, and disease etiology.

Initially the pre-miRNA is present as a long non-perfect double-stranded stem loop RNA that is further processed by Dicer into a siRNA-like duplex, comprising the mature guide strand (miRNA) and a similar-sized fragment known as the passenger strand (miRNA*). The miRNA and miRNA* may be derived from opposing arms of the pri-miRNA and pre-miRNA. miRNA* sequences may be found in libraries of cloned miRNAs but typically at lower frequency than the miRNAs.

Although initially present as a double-stranded species with miRNA*, the miRNA eventually becomes incorporated as a single-stranded RNA into a ribonucleoprotein complex known as the RNA-induced silencing complex (RISC). Various proteins can form the RISC, which can lead to variability in specificity for miRNA/miRNA* duplexes, binding site of the target gene, activity of miRNA (repress or activate), and which strand of the miRNA/miRNA* duplex is loaded into the RISC.

The RISC identifies target nucleic acids based on high levels of complementarity between the miRNA and the mRNA, especially by nucleotides 2-8 of the miRNA (referred as “seed sequence”).

A number of studies have looked at the base-pairing requirement between miRNA and its mRNA target for achieving efficient inhibition of translation (reviewed by Bartel 2004, Cell 116-281). Computational studies, analysing miRNA binding on whole genomes have suggested a specific role for bases 2-8 at the 5′ of the miRNA (also referred to as “seed sequence”) in target binding but the role of the first nucleotide, found usually to be “A” was also recognized (Lewis et al. 2005 Cell 120-15). Similarly, nucleotides 1-7 or 2-8 were used to identify and validate targets by Krek et al. (2005, Nat Genet 37-495). The target sites in the mRNA may be in the 5′ UTR, the 3′ UTR or in the coding region. Interestingly, multiple miRNAs may regulate the same mRNA target by recognizing the same or multiple sites. The presence of multiple miRNA binding sites in most genetically identified targets may indicate that the cooperative action of multiple RISCs provides the most efficient translational inhibition.

miRNAs may direct the RISC to downregulate gene expression by either of two mechanisms: mRNA cleavage or translational repression. The miRNA may specify cleavage of the mRNA if the mRNA has a certain degree of complementarity to the miRNA. When a miRNA guides cleavage, the cut is typically between the nucleotides pairing to residues 10 and 11 of the miRNA. Alternatively, the miRNA may repress translation if the miRNA does not have the requisite degree of complementarity to the miRNA. Translational repression may be more prevalent in animals since animals may have a lower degree of complementarity between the miRNA and binding site.

It should be noted that there may be variability in the 5′ and 3′ ends of any pair of miRNA and miRNA*. This variability may be due to variability in the enzymatic processing of Drosha and Dicer with respect to the site of cleavage. Variability at the 5′ and 3′ ends of miRNA and miRNA* may also be due to mismatches in the stem structures of the pri-miRNA and pre-miRNA. The mismatches of the stem strands may lead to a population of different hairpin structures. Variability in the stem structures may also lead to variability in the products of cleavage by Drosha and Dicer.

According to one embodiment, miRNAs can be processed independently of Dicer, e.g. by Argonaute 2.

It will be appreciated that the pre-miRNA sequence may comprise from 45-90, 60-80, 60-70, 80-120, 100-120 or 120-150 nucleotides while the pri-miRNA sequence may comprise from 45-30,000, 50-25,000, 100-20,000, 1,000-1,500 or 80-100 nucleotides. In certain embodiments, the silencing RNA is a pri-miRNA, a pre-miRNA, or a single stranded mature miRNA.

Antisense—Antisense is a single stranded RNA designed to prevent or inhibit expression of a gene by specifically hybridizing to its mRNA. Downregulation of a target RNA can be effected using an antisense polynucleotide capable of specifically hybridizing with an mRNA transcript encoding the target RNA.

### Transposable Element RNA

Transposable genetic elements (TEs) comprise a vast array of DNA sequences, all having the ability to move to new sites in genomes either directly by a cut-and-paste mechanism (transposons) or indirectly through an RNA intermediate (retrotransposons). TEs are divided into autonomous and non-autonomous classes depending on whether they have ORFs that encode proteins required for transposition. RNA-mediated gene silencing is one of the mechanisms in which the genome control TEs activity and deleterious effects derived from genome genetic and epigenetic instability.

The endogenous silencing sequence may not comprise a canonical (intrinsic) RNAi activity and/or may not be an active silencing sequence (e.g. is not a canonical RNA silencing molecule). Such endogenous silencing sequences include the following:

According to one embodiment, the endogenous silencing sequence is a transfer RNA (tRNA). The term “tRNA” refers to an RNA molecule that serves as the physical link between nucleotide sequence of nucleic acids and the amino acid sequence of proteins, formerly referred to as soluble RNA or sRNA. tRNA is typically about 76 to 90 nucleotides in length.

According to one embodiment, the endogenous silencing sequence is a ribosomal RNA (rRNA). The term “rRNA” refers to the RNA component of the ribosome i.e. of either the small ribosomal subunit or the large ribosomal subunit.

According to one embodiment, the endogenous silencing sequence is a small nuclear RNA (snRNA or U-RNA). The terms “sRNA” or “U-RNA” refer to the small RNA molecules found within the splicing speckles and Cajal bodies of the cell nucleus in eukaryotic cells. snRNA is typically about 150 nucleotides in length.

According to one embodiment, the endogenous silencing sequence is a small nucleolar RNA (snoRNA). The term “snoRNA” refers to the class of small RNA molecules that primarily guide chemical modifications of other RNAs, e.g. rRNAs, tRNAs and snRNAs. snoRNA is typically classified into one of two classes: the C/D box snoRNAs are typically about 70-120 nucleotides in length and are associated with methylation, and the H/ACA box snoRNAs are typically about 100-200 nucleotides in length and are associated with pseudouridylation.

Similar to snoRNAs are the scaRNAs (i.e. Small Cajal body RNA genes) which perform a similar role in RNA maturation to snoRNAs, but their targets are spliceosomal snRNAs and they perform site-specific modifications of spliceosomal snRNA precursors (in the Cajal bodies of the nucleus).

According to one embodiment, the endogenous silencing sequence is an extracellular RNA (exRNA). The term “exRNA” refers to RNA species present outside of the cells from which they were transcribed (e.g. exosomal RNA).

According to one embodiment, the endogenous silencing sequence is a long non-coding RNA (lncRNA). The term “lncRNA” or “long ncRNA” refers to non-protein coding transcripts typically longer than 200 nucleotides.

According to a specific embodiment, endogenous silencing sequences may include, microRNA (miRNA), piwi-interacting RNA (piRNA), short interfering RNA (siRNA), short-hairpin RNA (shRNA), phased small interfering RNA (phasiRNA), trans-acting siRNA (tasiRNA), small nuclear RNA (snRNA or URNA), transposable element RNA (e.g. autonomous and non-autonomous transposable RNA), transfer RNA (IRNA), small nucleolar RNA (snoRNA), Small Cajal body RNA (scaRNA), ribosomal RNA (rRNA), extracellular RNA (exRNA), repeat-derived RNA, and long non-coding RNA (lncRNA).

According to one embodiment, the endogenous silencing sequence is non-coding gene. Exemplary non-coding parts of the genome include, but are not limited to, genes of non-coding RNAs, enhancers and locus control regions, insulators, S/MAR sequences, non-coding pseudogenes, non-autonomous transposons and retrotransposons, and non-coding simple repeats of centromeric and telomeric regions of chromosomes.

According to one embodiment, the endogenous silencing sequence is positioned between genes, i.e. intergenic region. According to one embodiment, the endogenous silencing sequence is a coding gene (e.g. protein-coding gene).

The silencing insertions of the invention target different genes from the endogenous silencing sequences from which they are derived. In other words, the silencing insertion comprises a sequence encoding a silencing RNA that silences the expression of a target gene and the endogenous silencing sequence comprises a sequence encoding a silencing RNA that silences the expression of a second target gene that is different from the first target gene. The first and second target genes generally encode different proteins or RNA molecules. In certain embodiments, the first and second target genes have less than 80%, such as less than 70%, 60% or 50%, sequence identity.

### Promoters and Terminators

The silencing insertion of the invention comprises a promoter and a terminator to drive desired expression of the silencing RNA. As detailed above, the design of the silencing insertion includes analysis of the endogenous silencing sequence, its promoter, its terminator, and its expression profile to identify a sequence that is appropriate for modifying to target the desired target gene.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Examples 1 and 2 provides exemplary processes and databases useful for identifying appropriate promoter and terminator sequences.

In certain embodiments, the silencing insertion comprises a part of a promoter and/or a part of a terminator. It is not essential to precisely determine the limits of promoter and terminator sequences and effective expression of the silencing RNA may be achieved using part of the promoter and or part of the terminator.

In certain embodiments, the promoter is a constitutive promoter, a tissue specific promoter, an inducible promoter, a chimeric promoter, a developmentally regulated promoter, a biotic-condition specific promoter or an abiotic-condition specific promoter. The promoter may be a strong promoter or a weak promoter. Tissue specific promoters may drive expression in any appropriate tissue, such as seed, endosperm, embryo, flowers, anther, roots, young flowers, calli, shoot, leaves or meristem, or more than one of said tissues. Enhancer elements can stimulate transcription up to 1,000 fold from linked homologous or heterologous promoters. Enhancers are active when placed downstream or upstream from the transcription initiation site. In certain embodiments, the silencing insertion may comprise an enhancer from the endogenous silencing sequence. The silencing insertion may also comprise other sequences that control expression such as 5′UTR and 3′UTR sequences.

### Methods of Reducing the Expression of a Target Gene

The invention provides methods for reducing the expression of target genes comprising inserting silencing insertions into eukaryotic cells. Advantageously, the methods of the invention and the silencing insertions of the invention provide specific silencing whilst using endogenous sequence in its endogenous arrangement with minimal nucleotide modifications. The silencing insertions can be inserted into the eukaryotic cells using a variety of techniques available to the skilled person.

In preferred embodiments, the silencing insertion is inserted into the genome of a regenerating plant cell in a single delivery, thereby allowing generation of a plant with the insertion in every cell, and silencing of the target gene in every cell or selected cells, depending on the promoter, terminator or other regulatory sequence that is chosen.

In preferred embodiments, the silencing insertion is inserted by particle bombardment, protoplast transfection, electroporation or nanoparticle-mediated transfection. These techniques do not introduce exogenous transgenic (e.g. exogenous) sequence into the target cells, and are efficient.

In preferred embodiments, the silencing insertion expression cassette is introduced into the cells as linear DNA by DNA bombardment. In such embodiments the silencing insertion may be integrated by nonhomologous end joining (NHEJ).

In certain embodiments, the silencing insertion is introduced as a linear molecule with blunt ends. Such molecules can be produced using PCR with standard oligonucleotide primers and no further processing.

In certain embodiments, the silencing insertion is introduced as a linear molecule with sticky ends. Such molecules can be produced using PCR with oligonucleotide primers which contain unique restriction sites at their 5′ ends. Restriction digestion of PCR products with the corresponding high-fidelity restriction enzymes produces sticky ends.

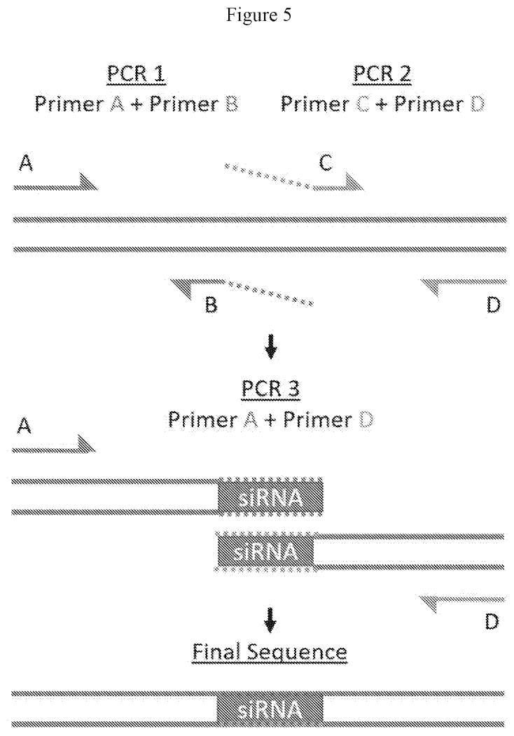

The silencing insertion expression cassette is preferably generated by chemical synthesis and amplified as a whole by high-fidelity PCR. Alternatively, the silencing insertion cassette may be generated by overlapping PCR reactions to introduce the relevant modifications into the sequence on which the insertion is based using genomic DNA as template (FIG. 5). Both these approaches constitute plasmid-free methods. After PCR amplification and before restriction digestion DNA might need to be concentrated by using centrifugal filters available as commercial kits. In certain embodiments, chemically stabilised molecules are used, such oligonucleotides containing stabilising 5′-phosphorothioate linkages to reduce exonuclease degradation.

In certain embodiments, the silencing insertion is introduced into the eukaryotic cell in a T-DNA cassette using Agrobacterium-mediated transformation, or the silencing insertion is within an inserted T-DNA cassette sequence, or the isolated silencing construct is a T-DNA cassette. Use of a T-DNA cassette achieves integration of the silencing insertion at high frequency in plant cells, and allows additional sequences to be integrated alongside the silencing insertion, such as selectable markers and endonucleases and guide RNAs, as discussed below. Such a T-DNA cassette may integrate into the genome in complete form, or the silencing insertion alone may integrate.

In certain such embodiments, the T-DNA cassette comprises a selectable marker, which can assist in selection of successful transformants. Exemplary selectable markers are discussed in the next section.

Various other methods can also be used to introduce the silencing insertion of the invention into eukaryotic cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation, microinjection, microparticle bombardment, infection with recombinant viral vectors. In addition, see U.S. Pat. Nos. 5,464,764 and 5,487,992 for positive-negative selection methods.

Thus, the delivery of nucleic acids may be introduced into a cell in embodiments of the invention by any method known to those of skill in the art, including, for example and without limitation: by transformation of protoplasts (Sec, e.g., U.S. Pat. No. 5,508,184); by desiccation/inhibition-mediated DNA uptake (See, e.g., Potrykus et al. (1985) Mol. Gen. Genet. 199:183-8); by electroporation (See, e.g., U.S. Pat. No. 5,384,253); by agitation with silicon carbide fibers (Sec, e.g., U.S. Pat. Nos. 5,302,523 and 5,464,765); by Agrobacterium-mediated transformation (See, e.g., U.S. Pat. Nos. 5,563,055, 5,591,616, 5,693,512, 5,824,877, 5,981,840, and 6,384,301); by acceleration of DNA-coated particles (Sec, e.g., U.S. Pat. Nos. 5,015,580, 5,550,318, 5,538,880, 6,160,208, 6,399,861, and 6,403,865) and by Nanoparticles, nanocarriers and cell penetrating peptides (WO201126644A2; WO2009046384A1; WO2008148223A1) in the methods to deliver DNA, RNA, Peptides and/or proteins or combinations of nucleic acids and peptides into cells. Preferably the method of insertion does not introduce any sequence in addition to the silencing insertion (and optional selectable marker), or does not introduce any exogenous sequence.

Other methods of transfection include the use of transfection reagents (e.g. Lipofectin, ThermoFisher), dendrimers (Kukowska-Latallo, J. F. et al., 1996, Proc. Natl. Acad. Sci. USA 93, 4897-902), cell penetrating peptides (Mãe et al., 2005, Internalisation of cell-penetrating peptides into tobacco protoplasts, Biochimica et Biophysica Acta 1669(2):101-7) or polyamines (Zhang and Vinogradov, 2010, Short biodegradable polyamines for gene delivery and transfection of brain capillary endothelial cells, J Control Release, 143(3):359-366).

According to a specific embodiment, for introducing DNA into cells (e.g. plant cells e.g. protoplasts) the method comprises polyethylene glycol (PEG)-mediated DNA uptake. For further details see Karesch et al. (1991) Plant Cell Rep. 9:575-578; Mathur et al. (1995) Plant Cell Rep. 14:221-226; Negrutiu et al. (1987) Plant Cell Mol. Biol. 8:363-373.

Introduction of nucleic acids to cells (e.g. eukaryotic cells) by viral infection offers several advantages over other methods such as lipofection and electroporation, since higher transfection efficiency can be obtained due to the infectious nature of viruses.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. For gene therapy, the preferred constructs are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers. Other than containing the necessary elements for the transcription and translation of the inserted coding sequence, the expression construct of some embodiments of the invention can also include sequences engineered to enhance stability, production, purification, yield or toxicity of the expressed peptide.

According to a specific embodiment, a bombardment method is used to introduce the silencing insertion into eukaryotic cells. According to one embodiment, the method is transient. Bombardment of eukaryotic cells is also taught by Uchida M et al., Biochim Biophys Acta. (2009) 1790(8):754-64, incorporated herein by reference.

According to one embodiment, plant cells may be transformed stably or transiently with the nucleic acid constructs of some embodiments of the invention. In stable transformation, the nucleic acid molecule of some embodiments of the invention is integrated into the plant genome and as such it represents a stable and inherited trait. In transient transformation, the nucleic acid molecule is expressed by the cell transformed but it is not integrated into the genome and as such it represents a transient trait.

There are various methods of introducing foreign genes into both monocotyledonous and dicotyledonous plants (Potrykus, I., Annu. Rev. Plant. Physiol., Plant. Mol. Biol. (1991) 42:205-225; Shimamoto et al., Nature (1989) 338:274-276).

The principal methods of causing stable integration of exogenous DNA into plant genomic DNA include two main approaches:
(i) Agrobacterium-mediated gene transfer: Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S. and Arntzen, C. J., Butterworth Publishers, Boston, Mass. (1989) p. 93-112.
(ii) direct DNA uptake: Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217; glass fibers or silicon carbide whisker transformation of cell cultures, embryos or callus tissue, U.S. Pat. No. 5,464,765 or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719.

The Agrobacterium system includes the use of plasmid vectors that contain defined DNA segments that integrate into the plant genomic DNA. Methods of inoculation of the plant tissue vary depending upon the plant species and the Agrobacterium delivery system. A widely used approach is the leaf disc procedure which can be performed with any tissue explant that provides a good source for initiation of whole plant differentiation. Horsch et al. in Plant Molecular Biology Manual A5, Kluwer Academic Publishers, Dordrecht (1988) p. 1-9. A supplementary approach employs the Agrobacterium delivery system in combination with vacuum infiltration. The Agrobacterium system is especially viable in the creation of transgenic dicotyledonous plants.

According to one embodiment, an Agrobacterium-free expression method is used to introduce foreign genes into plant cells. According to one embodiment, the Agrobacterium-free expression method is transient. According to a specific embodiment, a bombardment method is used to introduce foreign genes into plant cells. According to another specific embodiment, bombardment of a plant root is used to introduce foreign genes into plant cells.

The methods of the invention may comprise measuring the silencing specificity or efficiency of the silencing insertion, which may be determined by measuring an RNA or protein level of the target gene, or may be determined phenotypically.

Phenotypic determination may be affected by determination of at least one phenotype selected from the group consisting of a cell size, a growth rate/inhibition, a cell shape, a cell membrane integrity, a tumour size, a tumour shape, a tumour vascularization, a pigmentation of an organism, a size of an organism, a crop yield, metabolic profile, a fruit trait, a biotic stress resistance, an abiotic stress resistance, an infection parameter, and an inflammation parameter.

According to some embodiments of the invention, the silencing specificity or efficiency of the silencing insertion is determined genotypically.

According to some embodiments of the invention, the phenotype is determined prior to a genotype. According to some embodiments of the invention, the genotype is determined prior to a phenotype.

As used herein, reducing the expression of a target gene and silencing the expression of a target gene refer to the absence or observable reduction in the level of protein and/or mRNA product from the target gene. Thus, silencing of a target gene can be by 5%, 10%, 20%, 30%, 40%, 50%, 60%, 70%, 80%, 90%, 95% or 100% as compared to a target gene not targeted by the designed silencing insertion of the invention.

Successful insertion may be detected and modified cells selected at the phenotypic level, by detection of a molecular event, by detection of a fluorescent reporter, or by growth in the presence of selection (when a selectable marker is used). Example 5 provides exemplary identification and selection protocols.

According to one embodiment, selection of modified cells is performed by analysing the expression of the newly edited silencing RNA molecule (e.g. the presence of novel edited miRNA, siRNAs, piRNAs, tasiRNAs, etc).

According to one embodiment, selection of modified cells is performed by analysing a phenotypic trait influenced by the target gene, such as cell size, growth rate/inhibition, cell shape, cell membrane integrity, tumour size, tumour shape, tumour vascularization, pigmentation, size, infection parameters in an organism (such as viral load or bacterial load), plant leaf colouring, e.g. partial or complete loss of chlorophyll in leaves and other organs (bleaching), presence/absence of necrotic patterns, flower colouring, fruit traits (such as shelf life, firmness and flavour), growth rate, plant size (e.g. dwarfism), crop yield, biotic stress resistance (e.g. disease resistance, nematode mortality, beetle's egg laying rate, or other resistant phenotypes associated with any of bacteria, viruses, fungi, parasites, insects, weeds, and cultivated or native plants), crop yield, metabolic profile, fruit trait, biotic stress resistance, abiotic stress resistance (e.g. heat/cold resistance, drought resistance, salt resistance, resistance to allyl alcohol, or resistant to lack of nutrients e.g. Phosphorus (P)).

According to one embodiment, selection of modified cells is performed by analysing the silencing activity and/or specificity of the silencing insertion by measuring an RNA level of the target gene. This can be effected using any method known in the art, e.g. by Northern blotting, Nuclease Protection Assays, In Situ hybridization, quantitative RT-PCR or immunoblotting.

According to one embodiment, selection of modified cells is performed by analysing cells for the presence of the silencing insertion, which differs in sequence from the endogenous silencing sequence. Methods for detecting sequence alteration are well known in the art and include, but not limited to, DNA and RNA sequencing (e.g., next generation sequencing), electrophoresis, an enzyme-based mismatch detection assay and a hybridization assay such as PCR, RT-PCR, RNase protection, in-situ hybridization, primer extension, Southern blot, Northern Blot and dot blot analysis. Various methods used for detection of single nucleotide polymorphisms (SNPs) can also be used, such as PCR based T7 endonuclease, Heteroduplex and Sanger sequencing, or PCR followed by restriction digest to detect appearance or disappearance of unique restriction site/s.

Another method of validating the presence of a DNA editing event e.g., Indels comprises a mismatch cleavage assay that makes use of a structure selective enzyme (e.g. endonuclease) that recognizes and cleaves mismatched DNA.

According to one embodiment, selection of transformed cells is effected by flow cytometry (FACS) selecting transformed cells exhibiting fluorescence emitted by the fluorescent reporter. Following FACS sorting, positively selected pools of transformed eukaryotic cells, displaying the fluorescent marker are collected and an aliquot can be used for testing the DNA editing event as discussed above.

In cases where antibiotic selection marker was used, following transformation eukaryotic cells are cultivated in the presence of selection (e.g., antibiotic), e.g. in a cell culture or until the plant cells develop into colonies i.e., clones and micro-calli. A portion of the cells of the cell culture or of the calli is then analysed (validated) for the DNA editing event, as discussed above.

Positive eukaryotic cell clones may be stored (e.g., cryopreserved).

In preferred embodiments of the method of the invention, the silencing insertion is inserted into the genome of the eukaryotic cell in a construct that additionally comprises sequence encoding an RNA-guided DNA endonuclease and sequence encoding a guide RNA targeted to the endogenous silencing sequence. Similarly, the eukaryotic cell of the invention may preferably comprise in its genome a construct that comprises sequence encoding an RNA-guided DNA endonuclease, sequence encoding a guide RNA targeted to the endogenous silencing sequence, and a silencing insertion of the invention. Similarly, the silencing construct of the invention may comprise the silencing insertion and sequence encoding an RNA-guided DNA endonuclease and sequence encoding a guide RNA targeted to the endogenous silencing sequence. Preferably, the silencing construct is a T-DNA cassette. The advantages of such embodiments of the invention and those set out in the following paragraphs are further demonstrated in Example 6.

Use of RNA-guided DNA endonucleases and guide RNAs targeted to the endogenous silencing sequence as set out in the embodiments above allows additional modifications to be made to the eukaryotic cell. For example, the RNA-guided DNA endonuclease and guide RNA can introduce a modification into the endogenous silencing sequence, such as an insertion, deletion or insertion and deletion, which reduces or abolishes expression or activity of the endogenous silencing sequence. Therefore, the new silencing specificity of the silencing insertion is provided whilst the endogenous silencing sequence is knocked out, in a single transformation. Accordingly, in certain embodiments of the method of the invention, the RNA-guided DNA endonuclease and guide RNA introduce a modification into the endogenous silencing sequence, such as an insertion, deletion or insertion and deletion, which reduces or abolishes expression or activity of the endogenous silencing sequence. Also, in certain embodiments, the endogenous silencing sequence in the eukaryotic cell of the invention comprises a modification introduced by the RNA-guided DNA endonuclease and guide RNA, such as an insertion, deletion or insertion and deletion, which reduces or abolishes expression or activity of the endogenous silencing sequence. In certain embodiments, every endogenous silencing sequence in the cell, plant or animal is knocked out by an insertion, deletion or insertion and deletion.

Furthermore, use of RNA-guided DNA endonucleases and guide RNAs targeted to the endogenous silencing sequence as set out in the embodiments above allows the silencing insertion to be substituted for the endogenous silencing sequence by homology directed repair (HDR). This provides the new silencing specificity at the endogenous locus. The silencing insertion with the endonuclease and guide RNA could then be bred out to provide a plant or livestock animal that comprises the silencing insertion substituted at the location of the endogenous silencing sequence and that does not comprise the construct that was inserted into the genome of the eukaryotic cell. Such a plant or livestock animal will have minimal genetic changes, which is highly desirable, for example for consumers and regulators. Accordingly, in certain embodiments of the method of the invention, the RNA-guided DNA endonuclease and guide RNA mediate substitution of the endogenous silencing sequence with the silencing insertion in the eukaryotic cell, and optionally the method additionally comprises generating a plant or a livestock animal and breeding out the construct that was inserted into the genome, thereby generating a plant or livestock animal that comprises the silencing insertion substituted at the location of the endogenous silencing sequence and that does not comprise the construct that was inserted into the genome of the eukaryotic cell. In certain embodiments, every endogenous silencing sequence in the cell, plant or animal is substituted.

In certain embodiments, one or more endogenous silencing sequences in the cell plant or animal is knocked out by an insertion, deletion or insertion and deletion and one or more nous silencing sequences in the cell plant or animal is substituted for the silencing insertion.

Consistent with the embodiment described above, in a separate aspect of the invention, which may be combined with any embodiment described herein, there is provided a eukaryotic cell comprising in its genome:
(a) an inserted construct that comprises sequence encoding an RNA-guided DNA endonuclease, sequence encoding a guide RNA targeted to an endogenous silencing sequence, and a silencing insertion comprising a promoter or a part thereof, a sequence encoding a silencing RNA that silences the expression of a first target gene, and a terminator or a part thereof,
(b) an endogenous genome sequence that has been separated by the inserted construct, and
(c) an endogenous silencing sequence comprising a promoter, a sequence encoding a silencing RNA and a terminator, that has been substituted by the silencing insertion, wherein the silencing insertion and the endogenous silencing sequence prior to its substitution have over 95% sequence identity across the length of the insertion, and wherein the silencing RNA encoded by the endogenous silencing sequence is not active or silences the expression of a second target gene that is different from the first target gene.

In certain embodiments, the construct comprising the silencing insertion and the sequence encoding an RNA-guided DNA endonuclease and sequence encoding a guide RNA targeted to the endogenous silencing sequence may be integrated without modifying the endogenous silencing sequence. The method of the invention may then additionally comprise generating a plant or a livestock animal and breeding the plant or livestock animal for at least one generation until the RNA-guided DNA endonuclease and guide RNA mediate substitution of the endogenous silencing sequence with the silencing insertion.

The above embodiments provide an efficient product development approach, because integration of the silencing insertion with an endonuclease and guide RNA allows high-frequency generation of plants or livestock animals expressing the silencing insertion, whilst also providing the possibility of swapping the silencing insertion for the endogenous silencing locus, in a single transformation. The silencing insertion with the endonuclease and guide RNA can then be bred out to generate plants or animals with minimal genetic changes.

In additional aspects of the invention, which may be combined with any embodiment disclosed herein, there is provided a method of reducing the expression of a first target gene comprising introducing a silencing cassette into the genome of a eukaryotic cell,
wherein the silencing cassette comprises a promoter or a part thereof, a sequence encoding a silencing RNA that silences the expression of the first target gene, and a terminator or a part thereof,
wherein the silencing cassette has over 95% sequence identity across its length to an endogenous silencing sequence in the eukaryotic cell that comprises a promoter, a sequence encoding a silencing RNA and a terminator, and
wherein the silencing RNA encoded by the endogenous silencing sequence is not active or silences the expression of a second target gene that is different from the first target gene. In certain embodiments, the silencing cassette is substituted for the endogenous silencing sequence via the homology directed repair pathway. In certain embodiments of this aspect of the invention, the silencing cassette is introduced on a sequence that also encodes an RNA-guided DNA endonuclease and sequence encoding a guide RNA that are suitable for mediating introduction of the silencing cassette at the location of the endogenous silencing sequence via the homology directed repair pathway.

In certain embodiments, the silencing insertion or silencing cassette comprises homology arms 5′ of the promoter and 3′ of the terminator to aid HDR at the endogenous silencing sequence locus. Homology arms may be 100-2000 nucleotides in length, such as 100-1500, 100-1000, 200-1000, 200-750, 300-750, 350-750, 400-600 nucleotides in length, In certain such embodiments, the T-DNA cassette comprises a selectable marker, which can assist in selection of successful transformants. Exemplary selectable markers are discussed in the next section.

Suitable RNA-guided DNA endonucleases suitable for use in the above embodiments of the invention are generally “CRISPR-associated endonucleases” (or “Cas”), which refers to an endonuclease having an RNA-guided polynucleotide-editing activity. Such endonucleases are one of the components of the CRISPR/Cas system for genome editing, which uses at least one additional component, a “guide RNA” (gRNA). In some embodiments of the invention, the RNA-guided DNA endonucleases is a “Cas9 endonuclease” (or “Cas9”). According to some embodiments, the RNA-guided DNA endonuclease may be any Cas9 known in the art, such as, but not limited to, SpCas9, SaCas9, FnCas9, NmCas9, St1Cas9, BlatCas9 (Shota Nakade, Takashi Yamamoto & Tetsushi Sakuma (2017), Bioengineered 8:3, 265-273, and references therein). In other embodiments, the RNA-guided DNA endonuclease may be Cpf1, such as, but not limited to, AsCpf1 or LbCpf1 (Shota Nakade, Takashi Yamamoto & Tetsushi Sakuma (2017), Bioengineered, 8:3, 265-273, and references therein).

According to some embodiments, an RNA-guided DNA endonuclease suitable for use in the above embodiments of the invention is a “modified CRISPR-associated endonuclease” (or “modified Cas”). As used, herein a modified Cas refers to a Cas in which the catalytic domain has been altered and/or which are fused to additional domain. According to some embodiments, a “modified Cas” refers to a Cas which contains inactive catalytic domains (dead Cas, or dCas) and has no nuclease activity while still being able to bind to DNA based on gRNA specificity. According to some embodiments, a “modified Cas” refers to a Cas which has a nickase activity (“nCas9”), thus inducing a single strand break. In some embodiments, the modified CRISPR-associated endonuclease is a “modified Cas9 endonuclease”, possibly a catalytically inactive Cas9 (or “dCas9”) or a nickase Cas9 (“nCas9”). The dCas can be utilized as a platform for DNA transcriptional regulators to activate or repress gene expression by fusing the inactive enzyme to known regulatory domains. For example, the binding of dCas alone to a target sequence in genomic DNA can interfere with gene transcription. There are a number of publicly available tools available to help choose and/or design target sequences as well as lists of bioinformatically determined unique gRNAs for different genes in different species such as the Feng Zhang lab's Target Finder, the Michael Boutros lab's Target Finder “E-CRISP”, the RGEN Tools: “Cas-OFFinder”, the CasFinder: Flexible algorithm for identifying specific Cas9 targets in genomes and the CRISPR Optimal Target Finder.

In the context of the invention, modified Cas, such as dCas or nCas9, can also be used according to some embodiments together with other enzymes (possibly as a fusion protein) for base-editing. Base editing is a genome editing approach that uses components from CRISPR systems together with other enzymes to directly install point mutations into cellular DNA or RNA without making double-stranded DNA breaks. DNA base editors comprise a catalytically disabled nuclease fused to a nucleobase deaminase enzyme and, in some cases, a DNA glycosylase inhibitor. RNA base editors achieve analogous changes using components that target RNA. Base editors directly convert one base or base pair into another, enabling the efficient installation of point mutations in non-dividing cells without generating excess undesired editing byproducts (Rees and Liu (2018), “Base Editing: Precision Chemistry on the Genome and Transcriptome of Living Cells”, Nature Reviews Genetics, 19(12): 770-788). According to some embodiments, the modified Cas9 is an nCas fused to a base editor enzyme such as an adenosine or cytidine deaminase. Particular base editors contemplated include APOBEC, BE1, BE2, BE3, HF-BE3, BE4, BE4max, BE4-GAM, YE1-BE3, EE-BE3, YE-BE3, YEE-BE3, VQR-BE3, VRER-BE3, Sa-BE3, Sa-BE4, SaBE4-Gam, SaKKH-BE3, Cas12a-BE, Target-AID, Target-AID-NG, xBE3, cA3A-BE3, A3A-BE3, BE-PLUS, TAM, CRISPR-X, ABE7.9, ABE7.10, ABE7.10*, xABE, ABESa, VQR-ABE, VRER-ABE, SaKKH-ABE (Rees and Liu (2018), “Base Editing: Precision Chemistry on the Genome and Transcriptome of Living Cells”, Nature Reviews Genetics, 19(12): 770-788, and references therein).

The terms “guide RNA” or “gRNA” as used herein may be used interchangeably and refer to a polynucleotide which facilitates the specific targeting of an RNA-guided DNA endonuclease or CRISPR-associated endonuclease or a modified CRISPR-associated endonuclease to a target sequence. Therefore, the guide RNA comprises sequence suitable for targeting the endogenous silencing sequence. According to some embodiments, gRNAs can be chimeric/uni-molecular (comprising a single RNA molecule, also referred to as single guide RNA or sgRNA) or modular (comprising more than one separate RNA molecule, typically a crRNA and tracrRNA which may be linked, for example by duplexing). According to some embodiments, a gRNA is an sgRNA. The sgRNA is an RNA molecule which includes both the tracrRNA and crRNA (and a connecting loop). The sgRNA comprises a nucleotide sequence encoding the target homologous sequence (crRNA) and the endogenous bacterial RNA that links the crRNA to the Cas nuclease (tracrRNA) in a single chimeric transcript. This region of the crRNA, known as the variable region, confers the cutting specificity of the associated endonuclease, and is typically 20 nucleotides in length.

### Selectable Markers

In certain embodiments, the silencing insertion is adjacent to an additional sequence that is a selectable marker sequence. Such use of a selectable marker aids identification of successful transformants and increases the efficiency of generating cells with silenced genes.

Generally, the selectable marker will not be derived from the same endogenous sequence as the endogenous silencing sequence, and so will not be highly identical to sequence at or adjacent to the endogenous silencing sequence. Accordingly, the sequence identity between the silencing insertion and the endogenous sequence is calculated by excluding the adjacent selectable marker. In certain such embodiments, the selectable marker sequence has at least 90%, such as at least 92%, 94%, 96%, 98%, 99%, 99.5% or 99.9% sequence identity to a different endogenous sequence.

Generally, the selectable marker will include its own promoter and terminator. According to some embodiments, the selectable marker will include its naturally occurring promoter and terminator, or parts thereof. According to some embodiments, the selectable marker is an endogenous sequence of the eukaryotic cell, or a mutated version thereof (e.g. a dominant mutation), wherein the selectable marker includes its naturally occurring promotor and terminator, or parts thereof. In certain embodiments, the selectable marker and the silencing insertion will be arranged in a “head to head” conformation with their promoters transcribed in opposite directions. In alternative embodiments, the promoters will be transcribed in the same direction.

In preferred embodiments, the selectable marker sequence is endogenous to the eukaryotic cell. In especially preferred embodiments, the selectable marker is a mutant version of a sequence endogenous to the eukaryotic cell. In further especially preferred embodiments, the mutant version of the marker is the same as the marker in another eukaryotic cell of the same species. In other words, the mutant version of the marker occurs naturally in certain individuals. Any of such markers ensure that minimal exogenous genetic material is used to achieve gene silencing.

A preferred selectable marker for use in the invention is a mutated ALS gene that confers herbicide resistance to ALS inhibitors, such as, but not limited to chlorsulfuron. According to some embodiments, the mutated ALS gene occurs naturally, so can be introduced as a selectable marker linked with the silencing insertion without introducing exogenous genetic material. Exemplary such methods are provided in Example 3. A preferred ALS mutation is P197T.

In alternative embodiments, the marker is a selectable excisable element that can be excised out of the genome after successful transformants have been identified. In certain such embodiments the element comprises a selectable marker, such as antibiotic resistance gene, that can by excised by a gene editing agent. The selectable marker cassette is flanked by endogenous target sequences that are recognised and cleaved by gene editing factors, such as CRISPR/CAS9 or TALEN. Genomic excision of the selectable marker is achieved by introducing sequence-specific gene editing factor that cleaves the inserted flanking endogenous sequences.

In alternative embodiments, the marker, optionally the excisable marker, is an antibiotic selection marker. Examples of antibiotic selection markers that can be used are, neomycin phosphotransferase II (nptII) and hygromycin phosphotransferase (hpt). Additional marker genes which can be used in accordance with the present teachings include, but are not limited to, gentamycin acetyltransferase (accC3) resistance and bleomycin and phleomycin resistance genes. Further preferred markers include: mutant psbA that provides triazine-resistance, in particular G264S and I219V; and mutant enolpyruvylshikimate-3-phosphate synthase (EPSPS) that confer resistance to EPSP synthase inhibitors, in particular tryptophan 102 mutations, alanine 103 mutations and proline 106 mutations.

It will be appreciated that the enzyme NPTII inactivates by phosphorylation a number of aminoglycoside antibiotics such as kanamycin, neomycin, geneticin (or G418) and paromomycin. Of these, kanamycin, neomycin and paromomycin are used in a diverse range of plant species.

### Plants and Methods of Generating Plants and Plant Products

The methods and products of the invention are useful for silencing genes in eukaryotic cells, including plant cells. Plant cells with silenced expression of specific genes may be used to generate improved plants and plant products, which may have improved traits or be produced more efficiently.

Accordingly, the invention provides a plant or a part of plant, such as a seed, comprising a eukaryotic cell of the invention, and provides a method of growing a plant cell of the invention into a plant and optionally propagating the plant. The invention also provides methods of harvesting fruit or other plant products.

In certain embodiments, the methods of the invention may comprise additional breeding, which may comprise crossing or selfing.

In certain embodiments, the eukaryotic cell of the invention is a protoplast, such as a protoplast derived from any plant tissue e.g., fruit, flowers, roots, leaves, embryos, embryonic cell suspension, calli or seedling tissue. In certain embodiments, the plant cell is an embryogenic cell, such as a somatic embryogenic cell.

Following stable transformation with a silencing insertion of the invention, plant propagation may be exercised. The most common method of plant propagation is by seed. Regeneration by seed propagation, however, has the deficiency that due to heterozygosity there is a lack of uniformity in the crop, since seeds are produced by plants according to the genetic variances governed by Mendelian rules. Basically, each seed is genetically different and each will grow with its own specific traits. Therefore, it is preferred that the transformed plant be produced such that the regenerated plant has the identical traits and characteristics of the parent transgenic plant. Therefore, it is preferred that the transformed plant be regenerated by micropropagation which provides a rapid, consistent reproduction of the genetically identical transformed plants.

Micropropagation is a process of growing new generation plants from a single piece of tissue that has been excised from a selected parent plant or cultivar. This process permits the mass reproduction of plants having the desired trait. The new generated plants are genetically identical to, and have all of the characteristics of, the original plant. Micropropagation (or cloning) allows mass production of quality plant material in a short period of time and offers a rapid multiplication of selected cultivars in the preservation of the characteristics of the original transgenic or transformed plant. The advantages of cloning plants are the speed of plant multiplication and the quality and uniformity of plants produced.

Micropropagation is a multi-stage procedure that requires alteration of culture medium or growth conditions between stages. Thus, the micropropagation process involves four basic stages: Stage one, initial tissue culturing; stage two, tissue culture multiplication; stage three, differentiation and plant formation; and stage four, greenhouse culturing and hardening. During stage one, initial tissue culturing, the tissue culture is established and certified contaminant-free. During stage two, the initial tissue culture is multiplied until a sufficient number of tissue samples are produced to meet production goals. During stage three, the tissue samples grown in stage two are divided and grown into individual plantlets. At stage four, the transformed plantlets are transferred to a greenhouse for hardening where the plants' tolerance to light is gradually increased so that they can be grown in the natural environment.

Although stable transformation is presently preferred, transient transformation of leaf cells, meristematic cells or the whole plant is also envisaged by some embodiments of the invention.

Preferred plant products of the invention include seeds, particularly coffee beans and rice, and fruits, particularly bananas.

The eukaryotic cells of the invention may be further cultured and maintained, for example, in an undifferentiated state for extended periods of time or may be induced to differentiate into other cell types, tissues, organs or organisms as required.

Plant cells (e.g., protoplasts or cells in an Embryonic Cell Suspension, ECS) may be regenerated into whole plants first by growing into a group of plant cells that develops into a callus and then by regeneration of shoots (callogenesis) from the callus using plant tissue culture methods. Growth of protoplasts into callus and regeneration of shoots requires the proper balance of plant growth regulators in the tissue culture medium that must be customized for each species of plant.

Protoplasts may also be used for plant breeding, using a technique called protoplast fusion. Protoplasts from different species are induced to fuse by using an electric field or a solution of polyethylene glycol. This technique may be used to generate somatic hybrids in tissue culture.

Methods of protoplast regeneration are well known in the art. Several factors affect the isolation, culture, and regeneration of protoplasts, namely the genotype, the donor tissue and its pre-treatment, the enzyme treatment for protoplast isolation, the method of protoplast culture, the culture, the culture medium, and the physical environment. For a thorough review see Maheshwari et al. 1986 Differentiation of Protoplasts and of Transformed Plant Cells: 3-36. Springer-Verlag, Berlin.

The regenerated plants can be subjected to further breeding and selection as the skilled artisan sees fit.

Thus, embodiments of the invention further relate to plants, plant cells and processed product of plants comprising the silencing insertion of the invention.

According to one aspect of the invention, there is provided a method producing a plant or plant cell of some embodiments of the invention, comprising growing the plant or plant cell under conditions which allow propagation.

The term “plant” as used herein encompasses whole plants, a grafted plant, ancestors and progeny of the plants and plant parts, including seeds, shoots, stems, roots (including tubers), rootstock, scion, and plant cells, tissues and organs. The plant may be in any form including suspension cultures, embryos, meristematic regions, callus tissue, leaves, gametophytes, sporophytes, pollen, and microspores. Plants that may be useful in the methods of the invention include all plants which belong to the superfamily Viridiplantae, in particular monocotyledonous and dicotyledonous plants including a fodder or forage legume, ornamental plant, food crop, tree, or shrub selected from the list comprising Acacia spp., Acer spp., Actinidia spp., Aesculus spp., Agathis australis, Albizia amara, Alsophila tricolor, Andropogon spp., Arachis spp, Areca catechu, Astelia fragrans, Astragalus cicer, Baikiaca plurijuga, Betula spp., Brassica spp., Bruguiera gymnorrhiza, Burkea africana, Butca frondosa, Cadaba farinosa, Calliandra spp, Camellia sinensis, Cannabaceae, Cannabis indica, Cannabis, Cannabis sativa, Hemp, industrial Hemp, Capsicum spp., Cassia spp., Centroema pubescens, Chacoomeles spp., Cinnamomum cassia, Coffea arabica, Colophospermum mopane, Coronillia varia, Cotoneaster serotina, Crataegus spp., Cucumis spp., Cupressus spp., Cyathca dealbata, Cydonia oblonga, Cryptomeria japonica, Cymbopogon spp., Cynthea dealbata, Cydonia oblonga, Dalbergia monetaria, Davallia divaricata, Desmodium spp., Dicksonia squarosa, Dibeteropogon amplectens, Dioclea spp, Dolichos spp., Doryenium rectum, Echinochloa pyramidalis, Ehraffia spp., Eleusine coracana, Eragrestis spp., Erythrina spp., Eucalypfus spp., Euclea schimperi, Eulalia vi/losa, Pagopyrum spp., Feijoa sellowlana, Fragaria spp., Flemingia spp, Freycinetia banksli, Geranium thunbergii, GinAgo biloba, Glycine javanica, Gliricidia spp, Gossypium hirsutum, Grevillea spp., Guibourtia coleosperma, Hedysarum spp., Hemaffhia altissima, Heteropogon contoffus, Hordeum vulgare, Hyparrhenia rufa, Hypericum erectum, Hypeffhelia dissolute, Indigo incamata, Iris spp., Leptarrhena pyrolifolia, Lespediza spp., Lettuca spp., Leucaena leucocephala, Loudetia simplex, Lotonus bainesli, Lotus spp., Macrotyloma axillare, Malus spp., Manihot esculenta, Medicago saliva, Metasequoia glyptostroboides, Musa sapientum, banana, Nicotianum spp., Onobrychis spp., Ornithopus spp., Oryza spp., Peltophorum africanum, Pennisetum spp., Persea gratissima, Petunia spp., Phaseolus spp., Phoenix canariensis, Phormium cookianum, Photinia spp., Picca glauca, Pinus spp., Pisum sativam, Podocarpus totara, Pogonarthria fleckii, Pogonaffhria squarrosa, Populus spp., Prosopis cineraria, Pseudotsuga menziesii, Pterolobium stellatum, Pyrus communis, Quercus spp., Rhaphiolepsis umbellata, Rhopalostylis sapida, Rhus natalensis, Ribes grossularia, Ribes spp., Robinia pseudoacacia, Rosa spp., Rubus spp., Salix spp., Schyzachyrium sanguineum, Sciadopitys vefficillata, Sequoia sempervirens, Sequoiadendron giganteum, Sorghum bicolor, Spinacia spp., Sporobolus fimbriatus, Stiburus alopecuroides, Stylosanthos humilis, Tadchagi spp, Taxodium distichum, Themeda triandra, Trifolium spp., Triticum spp., Tsuga heterophylla, Vaccinium spp., Vicia spp., Vitis vinifera, Watsonia pyramidata, Zantedeschia aethiopica, Zea mays, amaranth, artichoke, asparagus, broccoli, Brussels sprouts, cabbage, canola, carrot, cauliflower, celery, collard greens, flax, kale, lentil, oilseed rape, okra, onion, potato, rice, soybean, straw, sugar beet, sugar cane, sunflower, tomato, squash tea, trees. Alternatively, algae and other non-Viridiplantae can be used for the methods of some embodiments of the invention.

According to a specific embodiment, the plant is a crop, a flower or a tree.

According to a specific embodiment, the plant is a woody plant species e.g., Actinidia chinensis (Actinidiaceae), Manihotesculenta (Euphorbiaceae), Firiodendron tulipifera (Magnoliaceae), Populus (Salicaceae), Santalum album (Santalaceae), Ulmus (Ulmaceae) and different species of the Rosaceae (Malus, Prunus, Pyrus) and the Rutaceae (Citrus, Microcitrus), Gymnospermae e.g., Picea glauca and Pinus taeda, forest trees (e.g., Betulaceae, Fagaceae, Gymnospermae and tropical tree species), fruit trees, shrubs or herbs, e.g., (banana, cocoa, coconut, coffee, date, grape and tea) and oil palm.

According to preferred embodiments, the plant is of a tropical crop e.g., coffee, macadamia, banana, pineapple, taro, papaya, mango, barley, beans, cassava, chickpea, cocoa (chocolate), cowpea, maize (corn), millet, rice, sorghum, sugarcane, sweet potato, tobacco, taro, tea, yam. In especially preferred embodiments, the crop is coffee, rice or banana.

“Grain,” “seed,” or “bean,” refers to a flowering plant's unit of reproduction, capable of developing into another such plant. As used herein, the terms are used synonymously and interchangeably.

According to a specific embodiment, the plant is a plant cell e.g., plant cell in an embryonic cell suspension.

According to a specific embodiment, the plant comprises a plant cell generated by the method of some embodiments of the invention.

The term “crossing” as used herein refers to the fertilization of female plants (or gametes) by male plants (or gametes). The term “gamete” refers to the haploid reproductive cell (egg or sperm) produced in plants by mitosis from a gametophyte and involved in sexual reproduction, during which two gametes of opposite sex fuse to form a diploid zygote. The term generally includes reference to a pollen (including the sperm cell) and an ovule (including the ovum). “Crossing” therefore generally refers to the fertilization of ovules of one individual with pollen from another individual, whereas “selfing” refers to the fertilization of ovules of an individual with pollen from the same individual. Crossing is widely used in plant breeding and results in a mix of genomic information between the two plants crossed, one chromosome set from the mother and one chromosome set from the father. This will result in a new combination of genetically inherited traits.

As mentioned above, the plant may be crossed in order to obtain a plant devoid of undesired factors e.g. DNA editing agent (e.g. endonuclease).

According to one embodiment, the plant is non-genetically modified (non-GMO) plant.

According to one embodiment, the plant is a genetically modified (GMO) plant.

According to one embodiment, there is provided a seed of the plant generated according to the method of some embodiments of the invention.

### Livestock Animals and Methods of Generating Animals and Animal Products

The methods and products of the invention are useful for silencing genes in eukaryotic cells, including animal cells and particularly livestock animal cells. Animal cells with silenced expression of specific genes may be used to generate improved animals and animal products, which may have improved traits or be produced more efficiently.

Accordingly, the invention provides an animal or an animal product, such as meat, milk, eggs, hide or wool, comprising a eukaryotic cell of the invention, and provides a method of growing an animal cell of the invention into an animal and optionally breeding the animal. The invention also provides methods of generating animal products from said animal.

In certain embodiments, the methods of the invention may comprise additional breeding, which may comprise crossing or selfing.

As mentioned above, the animal may be crossed in order to obtain an animal devoid of undesired factors e.g. DNA editing agent (e.g. endonuclease).

According to one embodiment, the animal is non-genetically modified (non-GMO) animal.

According to one embodiment, the animal is a genetically modified (GMO) animal.

### Target Genes and Traits

The present invention can be used to silence a great range of different genes to achieve various effects. The invention is particularly useful for modulation of endogenous gene expression to provide improved traits and to protect organisms against different biotic and abiotic stresses such as e.g. cancer, viruses, insects, fungi, nematodes, heat, drought, starvation etc. This section provides preferred target genes that are silenced by the silencing RNA encoded by the silencing insertion.

In certain embodiments, silencing a gene according to the present invention provides a plant with increased stress tolerance, increased yield, increased growth rate or increased yield quality.

In certain embodiments, silencing a gene according to the present invention provides an animal with increased yield, increased growth rate, or increased quality.

In certain embodiments, the target gene silenced by the silencing insertion is exogenous to the eukaryotic cell. In such a case, the gene is not naturally part of the eukaryotic cell genome (i.e. which expresses the silencing insert). Exemplary exogenous target genes include, genes associated with an infectious disease agent, such as a gene of a pathogen (e.g. an insect, a virus, a bacteria, a fungi, a nematode), as further discussed herein below.

According to some embodiments of the invention, the target gene silenced by the silencing insertion is selected from the group consisting of a housekeeping gene, a dominant gene, a gene comprising a high copy number and a gene associated with cell apoptosis.

In certain embodiments, the first target gene targeted by the silencing insert is a homeolog of the second target gene targeted by the endogenous silencing sequence.

The phrase “stress tolerance” as used herein refers to the ability of a plant to endure a biotic or abiotic stress without suffering a substantial alteration in metabolism, growth, productivity and/or viability.

The phrase “abiotic stress” as used herein refers to the exposure of a plant, plant cell, or the like, to a non-living (“abiotic”) environmental, physical or chemical agent that has an adverse effect on metabolism, growth, development, propagation, or survival of the plant (collectively, “growth”). An abiotic stress can be imposed on a plant due, for example, to an environmental factor such as water (e.g., flooding, drought, or dehydration), anaerobic conditions (e.g., a lower level of oxygen or high level of CO₂), abnormal osmotic conditions (e.g. osmotic stress), salinity, or temperature (e.g., hot/heat, cold, freezing, or frost), an exposure to pollutants (e.g. heavy metal toxicity), anaerobiosis, nutrient deficiency (e.g., nitrogen deficiency or limited nitrogen), atmospheric pollution or UV irradiation.

The phrase “biotic stress” as used herein refers to the exposure of a plant, plant cell, or the like, to a living (“biotic”) organism, yet including viruses, that has an adverse effect on metabolism, growth, development, propagation, yield or survival of the plant (collectively, “growth”). Biotic stress can be caused by, for example, bacteria, viruses, fungi, parasites, beneficial and harmful insects, weeds, and cultivated or native plants.

The phrase “yield” or “plant yield” as used herein refers to increased plant growth (growth rate), increased crop growth, increased biomass, and/or increased plant product production (including grain, fruit, seeds, etc.).

According to one embodiment, in order to generate a plant with increased stress tolerance, increased yield, increased growth rate or increased yield quality, the silencing insertion is designed to target a gene of the plant conferring sensitivity to stress, decreased yield, decreased growth rate or decreased yield quality.

According to one embodiment, exemplary susceptibility plant genes to be targeted (e.g. knocked down) include, but are not limited to, the susceptibility S-genes, such as those residing at genetic loci known as MLO (Mildew Locus O).

According to one embodiment, the plants generated by the present method comprise increased stress tolerance, increased yield, increased yield quality, increased growth rate, by at least about 10%, 20%, 30%, 40%, 50%, 60%, 70%, 80%, 90%, 95% or 100% as compared to plants not generated by the present methods.

Any method known in the art for assessing increased stress tolerance may be used in accordance with the present invention. Exemplary methods of assessing increased stress tolerance include, but are not limited to, downregulation of PagSAP1 in poplar for increased salt stress tolerance as described in Yoon, SK., Bac, EK., Lee, H. et al. Trees (2018) 32: 823.), and increased drought tolerance in tomato by downregulation of SlbZIP38 (Pan Y et al. Genes 2017, 8, 402; doi: 10.3390/genes8120402, incorporated herein by reference.

Any method known in the art for assessing increased yield may be used in accordance with the present invention. Exemplary methods of assessing increased yield include, but are not limited to, reduced DST expression in rice as described in Ar-Rafi Md. Faisal, et al, AJPS>Vol. 8 No. 9, August 2017 DOI: 10.4236/ajps.2017.89149; and downregulation of BnFTA in canola resulted in increased yield as described in Wang Y et al., Mol Plant. 2009 January; 2(1): 191-200. doi: 10.1093/mp/ssn088), both incorporated herein by reference.

Any method known in the art for assessing increased growth rate may be used in accordance with the present invention. Exemplary methods of assessing increased growth rate include, but are not limited to, reduced expression of BIG BROTHER in Arabidopsis or GA2-OXIDASE results in enhance growth and biomass as described in Marcelo de Freitas Lima et al. Biotechnology Research and Innovation (2017)1,14--25, incorporated herein by reference.

Any method known in the art for assessing increased yield quality may be used in accordance with the present invention. Exemplary methods of assessing increased yield quality include, but are not limited to, down regulation of OsCKX2 in rice results in production of more tillers, more grains, and the grains were heavier as described in Yeh S_Y et al. Rice (N Y). 2015; 8: 36; and reduce OMT levels in many plants, which result in altered lignin accumulation, increase the digestibility of the material for industry purposes as described in Verma S R and Dwivedi U N, South African Journal of Botany Volume 91, March 2014, Pages 107-125, both incorporated herein by reference.

According to one embodiment, the method further enables generation of a plant comprising increased sweetness, increased sugar content, increased flavour, improved ripening control, increased water stress tolerance, increased heat stress tolerance, and increased salt tolerance. One of skill in the art will know how to utilize the methods described herein to choose target gene sequences for silencing.

According to one embodiment, there is provided a method of generating a pathogen or pest tolerant or resistant plant, the method comprising: (a) breeding the plant of some embodiments of the invention, and (b) selecting for progeny plants that are pathogen or pest tolerant or resistant.

According to one embodiment, the target gene confers sensitivity to a pathogen or a pest. According to one embodiment, the target gene is a gene of a pathogen. According to one embodiment, the target gene a gene of a pest.

As used herein the term “pathogen” refers to an organism that negatively affect plants by colonizing, damaging, attacking, or infecting them. Thus, pathogen may affect the growth, development, reproduction, harvest or yield of a plant. This includes organisms that spread disease and/or damage the host and/or compete for host nutrients. Plant pathogens include, but are not limited to, fungi, oomycetes, bacteria, viruses, viroids, virus-like organisms, phytoplasmas, protozoa, nematodes, insects and parasitic plants. According to some embodiments, the first target gene targeted by the silencing insertion is a pathogen gene.

Non-limiting examples of pathogens include, but are not limited to, Roundheaded Borer such as long horned borers; psyllids such as red gum lerp psyllids (Glycaspis brimblecombei), blue gum psyllid, spotted gum lerp psyllids, lemon gum lep psyllids; tortoise beetles; snout beetles; leaf beetles; honey fungus; Thaumastocoris peregrinus; sessile gall wasps (Cynipidae) such as Leptocybe invasa, Ophelimus maskelli and Selitrichodes globules; Foliage-feeding caterpillars such as Omnivorous looper and Orange tortrix; Glassy-winged sharpshooter; and Whiteflies such as Giant whitefly. Other non-limiting examples of pathogens include Aphids such as Chaitophorus spp., Cloudywinged cottonwood and Periphyllus spp.; Armored scales such as Oystershell scale and San Jose scale; Carpenterworm; Clearwing moth borers such as American hornet moth and Western poplar clearwing; Flatheaded borers such as Bronze birch borer and Bronze poplar borer; Foliage-feeding caterpillars such as Fall webworm, Fruit-tree leafroller, Redhumped caterpillar, Satin moth caterpillar, Spiny elm caterpillar, Tent caterpillar, Tussock moths and Western tiger swallowtail; Foliage miners such as Poplar shield bearer; Gall and blister mites such as Cottonwood gall mite; Gall aphids such as Poplar petiolegall aphid; Glassy-winged sharpshooter; Leaf beetles and flea beetles; Mealybugs; Poplar and willow borer; Roundheaded borers; Sawflies; Soft scales such as Black scale, Brown soft scale, Cottony maple scale and European fruit lecanium; Treehoppers such as Buffalo treehopper; and True bugs such as Lace bugs and Lygus bugs.

Other non-limiting examples of viral plant pathogens include, but are not limited to Species: Pea early-browning virus (PEBV), Genus: Tobravirus. Species: Pepper ringspot virus (PepRSV), Genus: Tobravirus. Species: Watermelon mosaic virus (WMV), Genus: Potyvirus and other viruses from the Potyvirus Genus. Species: Tobacco mosaic virus Genus (TMV), Tobamovirus and other viruses from the Tobamovirus Genus. Species: Potato virus X Genus (PVX), Potexvirus and other viruses from the Potexvirus Genus. Thus the present teachings envisage targeting of RNA as well as DNA viruses (e.g. Geminivirus or Bigeminivirus). Geminiviridae viruses which may be targeted include, but are not limited to, Abutilon mosaic bigeminivirus, Ageratum yellow vein bigeminivirus, Bean calico mosaic bigeminivirus, Bean golden mosaic bigeminivirus, Bhendi yellow vein mosaic bigeminivirus, Cassava African mosaic bigeminivirus, Cassava Indian mosaic bigeminivirus, Chino del tomaté bigeminivirus, Cotton leaf crumple bigeminivirus, Cotton leaf curl bigeminivirus, Croton yellow vein mosaic bigeminivirus, Dolichos yellow mosaic bigeminivirus, Euphorbia mosaic bigeminivirus, Horsegram yellow mosaic bigeminivirus, Jatropha mosaic bigeminivirus, Lima bean golden mosaic bigeminivirus, Melon leaf curl bigeminivirus, Mung bean yellow mosaic bigeminivirus, Okra leaf-curl bigeminivirus, Pepper hausteco bigeminivirus, Pepper Texas bigeminivirus, Potato yellow mosaic bigeminivirus, Rhynchosia mosaic bigeminivirus, Serrano golden mosaic bigeminivirus, Squash leaf curl bigeminivirus, Tobacco leaf curl bigeminivirus, Tomato Australian leafcurl bigeminivirus, Tomato golden mosaic bigeminivirus, Tomato Indian leafcurl bigeminivirus, Tomato leaf crumple bigeminivirus, Tomato mottle bigeminivirus, Tomato yellow leaf curl bigeminivirus, Tomato yellow mosaic bigeminivirus, Watermelon chlorotic stunt bigeminivirus and Watermelon curly mottle bigeminivirus.

In preferred embodiments, the viral plant pathogen is Turnip Mosaic Virus (TuMV). In preferred such embodiments, the silencing RNA is a miRNA, for example a 22 nt miRNA.

As used herein the term “pest” refers to an organism which directly or indirectly harms the plant. A direct effect includes, for example, feeding on the plant leaves. Indirect effect includes, for example, transmission of a disease agent (e.g. a virus, bacteria, etc.) to the plant. In the latter case the pest serves as a vector for pathogen transmission.

According to one embodiment, the pest is an invertebrate organism.

Exemplary pests include, but are not limited to, insects, nematodes, snails, slugs, spiders, caterpillars, scorpions, mites, ticks, fungi, and the like.

Insect pests include, but are not limited to, insects selected from the orders Coleoptera (e.g. beetles), Diptera (e.g. flies, mosquitoes), Hymenoptera (e.g. sawflies, wasps, bees, and ants), Lepidoptera (e.g. butterflies and moths), Mallophaga (e.g. lice, e.g. chewing lice, biting lice and bird lice), Hemiptera (e.g. true bugs), Homoptera including suborders Sternorrhyncha (e.g. aphids, whiteflies, and scale insects), Auchenorrhyncha (e.g. cicadas, leafhoppers, treehoppers, planthoppers, and spittlebugs), and Colcorrhyncha (e.g. moss bugs and beetle bugs), Orthroptera (e.g. grasshoppers, locusts and crickets, including katydids and wetas), Thysanoptera (e.g. Thrips), Dermaptera (e.g. Earwigs), Isoptera (e.g. Termites), Anoplura (e.g. Sucking lice), Siphonaptera (e.g. Flea), Trichoptera (e.g. caddisflies), etc.

Insect pests of the invention include, but are not limited to, Maize: Ostrinia nubilalis, European corn borer; Agrotis ipsilon, black cutworm; Helicoverpa zea, corn carworm; Spodoptera frugiperda, fall armyworm; Diatraea grandiosella, southwestern corn borer; Elasmopalpus lignosellus, lesser cornstalk borer; Diatraea saccharalis, surgarcane borer; Diabrotica virgifera, western corn rootworm; Diabrotica longicornis barberi, northern corn rootworm; Diabrotica undecimpunctata howardi, southern corn rootworm; Melanotus spp., wireworms; Cyclocephala borealis, northern masked chafer (white grub); Cyclocephala immaculata, southern masked chafer (white grub); Popillia japonica, Japanese beetle; Chaetocnema pulicaria, corn flea beetle; Sphenophorus maidis, maize billbug; Rhopalosiphum maidis, corn leaf aphid; Anuraphis maidiradicis, corn root aphid; Blissus leucopterus leucopterus, chinch bug; Melanoplus femurrubrum, redlegged grasshopper; Melanoplus sanguinipes, migratory grasshopper; Hylemya platura, seedcorn maggot; Agromyza parvicornis, corn blot leafminer; Anaphothrips obscrurus, grass thrips; Solenopsis milesta, thief ant; Tetranychus urticae, twospotted spider mite; Sorghum: Chilo partellus, sorghum borer; Spodoptera frugiperda, fall armyworm; Helicoverpa zea, corn carworm; Elasmopalpus lignosellus, lesser cornstalk borer; Feltia subterranea, granulate cutworm; Phyllophaga crinita, white grub; Eleodes, Conoderus, and Aeolus spp., wireworms; Oulema melanopus, cereal leaf beetle; Chaetocnema pulicaria, corn flea beetle; Sphenophorus maidis, maize billbug; Rhopalosiphum maidis; corn leaf aphid; Sipha flava, yellow sugarcane aphid; Blissus leucopterus leucopterus, chinch bug; Contarinia sorghicola, sorghum midge; Tetranychus cinnabarinus, carmine spider mite; Tetranychus urticae, twospotted spider mite; Wheat: Pseudaletia unipunctata, army worm; Spodoptera frugiperda, fall armyworm; Elasmopalpus lignosellus, lesser cornstalk borer; Agrotis orthogonia, western cutworm; Elasmopalpus lignosellus, lesser cornstalk borer; Oulema melanopus, cereal leaf beetle; Hypera punctata, clover leaf weevil; Diabrotica undecimpunctata howardi, southern corn rootworm; Russian wheat aphid; Schizaphis graminum, greenbug; Macrosiphum avenae, English grain aphid; Melanoplus femurrubrum, redlegged grasshopper; Melanoplus differentialis, differential grasshopper; Melanoplus sanguinipes, migratory grasshopper; Mayetiola destructor, Hessian fly; Sitodiplosis mosellana, wheat midge; Meromyza americana, wheat stem maggot; Hylemya coarctate, wheat bulb fly; Frankliniella fusca, tobacco thrips; Cephus cinctus, wheat stem sawfly; Aceria tulipae, wheat curl mite; Sunflower: Suleima helianthana, sunflower bud moth; Homoeosoma electellum, sunflower moth; zygogramma exclamationis, sunflower beetle; Bothyrus gibbosus, carrot beetle; Neolasioptera murtfeldtiana, sunflower seed midge; Cotton: Heliothis virescens, cotton budworm; Helicoverpa zea, cotton bollworm; Spodoptera exigua, beet armyworm; Pectinophora gossypiella, pink bollworm; Anthonomus grandis, boll weevil; Aphis gossypii, cotton aphid; Pseudatomoscelis seriatus, cotton fleahopper; Trialeurodes abutilonca, bandedwinged whitefly; Lygus lincolaris, tarnished plant bug; Melanoplus femurrubrum, redlegged grasshopper; Melanoplus differentialis, differential grasshopper; Thrips tabaci, onion thrips; Franklinkiella fusca, tobacco thrips; Tetranychus cinnabarinus, carmine spider mite; Tetranychus urticae, twospotted spider mite; Rice: Diatraea saccharalis, sugarcane borer; Spodoptera frugiperda, fall armyworm; Helicoverpa zea, corn carworm; Colaspis brunnea, grape Colaspis; Lissorhoptrus oryzophilus, rice water weevil; Sitophilus oryzae, rice weevil; Nephotettix nigropictus, rice leafhopper; Blissus leucopterus leucopterus, chinch bug; Acrosternum hilare, green stink bug; Soybean: Pseudoplusia includens, soybean looper; Anticarsia gemmatalis, velvetbean caterpillar; Plathypena scabs, green cloverworm; Ostrinia nubilalis, European corn borer; Agrotis ipsilon, black cutworm; Spodoptera exigua, beet armyworm; Heliothis virescens, cotton budworm; Helicoverpa zea, cotton bollworm; Epilachna varivestis, Mexican bean beetle; Myzus persicae, green peach aphid; Empoasca fabae, potato leafhopper; Acrosternum hilare, green stink bug; Melanoplus femurrubrum, redlegged grasshopper; Melanoplus differentialis, differential grasshopper; Hylemya platura, seedcorn maggot; Sericothrips variabilis, soybean thrips; Thrips tabaci, onion thrips; Tetranychus turkestani, strawberry spider mite; Tetranychus urticae, twospotted spider mite; Barley: Ostrinia nubilalis, European corn borer; Agrotis ipsilon, black cutworm; Schizaphis graminum, greenbug; Blissus leucopterus leucopterus, chinch bug; Acrosternum hilare, green stink bug; Euschistus servus, brown stink bug; Delia platura, seedcorn maggot; Mayetiola destructor, Hessian fly; Petrobia latens, brown wheat mite; Oil Seed Rape: Brevicoryne brassicae, cabbage aphid; Phyllotreta cruciferae, Flea beetle; Mamestra configurata, Bertha armyworm; Plutella xylostella, Diamond-back moth; Delia ssp., Root maggots. According to one embodiment, the pathogen is a nematode. Exemplary nematodes include, but are not limited to, the burrowing nematode (Radopholus similis), Caenorhabditis elegans, Radopholus arabocoffeae, Pratylenchus coffeae, root-knot nematode (Meloidogyne spp.), cyst nematode (Heterodera and Globodera spp.), root lesion nematode (Pratylenchus spp.), the stem nematode (Ditylenchus dipsaci), the pine wilt nematode (Bursaphelenchus xylophilus), the reniform nematode (Rotylenchulus reniformis), Xiphinema index, Nacobbus aberrans and Aphelenchoides besseyi.

According to one embodiment, the pathogen is a fungus. Exemplary fungi include, but are not limited to, Fusarium oxysporum, Leptosphaeria maculans (Phoma lingam), Sclerotinia sclerotiorum, Pyricularia grisea, Gibberella fujikuroi (Fusarium moniliforme), Magnaporthe oryzae, Botrytis cinereal, Puccinia spp., Fusarium graminearum, Blumeria graminis, Mycosphaerella graminicola, Colletotrichum spp., Ustilago maydis, Melampsora lini, Phakopsora pachyrhizi and Rhizoctonia solani.

According to a specific embodiment, the pest is an ant, a bee, a wasp, a caterpillar, a beetle, a snail, a slug, a nematode, a bug, a fly, a whitefly, a mosquito, a grasshopper, an earwig, an aphid, a scale, a thrip, a spider, a mite, a psyllid, and a scorpion.

Preferably, silencing of the pathogen or pest gene results in the suppression, control, and/or killing of the pathogen or pest which results in limiting the damage that the pathogen or pest causes to the plant. Controlling a pest includes, but is not limited to, killing the pest, inhibiting development of the pest, altering fertility or growth of the pest in such a manner that the pest provides less damage to the plant, decreasing the number of offspring produced, producing less fit pests, producing pests more susceptible to predator attack, or deterring the pests from eating the plant.

According to one embodiment, an exemplary plant gene to be targeted includes the gene eIF4E which confers sensitivity to viral infection in cucumber.

Identification of plant or pathogen target genes to be silenced may be achieved using any method known in the art such as by routine bioinformatics analysis.

According to one embodiment, the silencing insertion targets the nematode pathogen Radopholus similis genes Calreticulin13 (CRT) or collagen 5 (col-5).

According to one embodiment, the silencing insertion targets the fungi pathogen Fusarium oxysporum genes FOW2, FRP1, and OPR.

According to one embodiment, the pathogen gene includes, for example, vacuolar ATPase (vATPase), dvssj1 and dvssj2, α-tubulin or snf7.

According to a specific embodiment, when the plant is a Brassica napus (rapeseed), the target gene is a gene of Leptosphaeria maculans (Phoma lingam) (causing e.g. Phoma stem canker) (e.g. as set forth in GenBank Accession No: AM933613.1); a gene of Flea beetle (Phyllotreta vittula or Chrysomelidae, e.g. as set forth in GenBank Accession No: KT959245.1); or a gene of by Sclerotinia sclerotiorum (causing e.g. Sclerotinia stem rot) (e.g. as set forth in GenBank Accession No: NW_001820833.1).

According to a specific embodiment, when the plant is a Citrus x sinensis (Orange), the target gene is a gene of Citrus Canker (CCK) (e.g. as set forth in GenBank Accession No: AE008925); a gene of Candidatus Liberibacter spp. (causing e.g. Citrus greening disease) (e.g. as set forth in GenBank Accession No: CP001677.5); or a gene of Armillaria root rot (e.g. as set forth in GenBank Accession No: KY389267.1).

According to a specific embodiment, when the plant is a Elaeis guineensis (Oil palm), the target gene is a gene of Ganoderma spp. (causing e.g. Basal stem rot (BSR) also known as Ganoderma butt rot) (e.g. as set forth in GenBank Accession No: U56128.1), a gene of Nettle Caterpillar or a gene of any one of Fusarium spp., Phytophthora spp., Pythium spp., Rhizoctonia solani (causing e.g. Root rot).

According to a specific embodiment, when the plant is a Fragaria vesca (Wild strawberry), the target gene is a gene of Verticillium dahlia (causing e.g. Verticillium Wilt) (e.g. as set forth in GenBank Accession No: DS572713.1); or a gene of Fusarium oxysporum f. sp. fragariae (causing e.g. Fusarium wilt) (e.g. as set forth in GenBank Accession No: KR855868.1);

According to a specific embodiment, when the plant is a Glycine max (Soybean), the target gene is a gene of P. pachyrhizi (causing e.g. Soybean rust, also known as Asian rust) (e.g. as set forth in GenBank Accession No: DQ026061.1); a gene of Soybean Aphid (e.g. as set forth in GenBank Accession No: KJ451424.1); a gene of Soybean Dwarf Virus (SbDV) (e.g. as set forth in GenBank Accession No: NC_003056.1); or a gene of Green Stink Bug (Acrosternum hilare) (e.g. as set forth in GenBank Accession No: NW_020110722.1).

According to a specific embodiment, when the plant is a Gossypium raimondii (Cotton), the target gene is a gene of Fusarium oxysporum f. sp. vasinfectum (causing e.g. Fusarium wilt) (e.g. as set forth in GenBank Accession No: JN416614.1); a gene of Soybean Aphid (e.g. as set forth in GenBank Accession No: KJ451424.1); or a gene of Pink bollworm (Pectinophora gossypiella) (e.g. as set forth in GenBank Accession No: KU550964.1).

According to a specific embodiment, when the plant is a Oryza sativa (Rice), the target gene is a gene of Pyricularia grisea (causing e.g. Rice Blast) (e.g. as set forth in GenBank Accession No: AF027979.1); a gene of Gibberella fujikuroi (Fusarium moniliforme) (causing e.g. Bakanae Disease) (e.g. as set forth in GenBank Accession No: AY862192.1); or a gene of a Stem borer, e.g. Scirpophaga incertulas Walker-Yellow Stem Borer, S. innota Walker—White Stem Borer, Chilo suppressalis Walker—Striped Stem Borer, Sesa-mia inferens Walker—Pink Stem Borer (e.g. as set forth in GenBank Accession No: KF290773.1).

According to a specific embodiment, when the plant is a Solanum lycopersicum (Tomato), the target gene is a gene of Phytophthora infestans (causing e.g. Late blight) (e.g. as set forth in GenBank Accession No: AY855210.1); a gene of a whitefly Bemisia tabaci (e.g. Gennadius, e.g. as set forth in GenBank Accession No: KX390870.1); or a gene of Tomato yellow leaf curl geminivirus (TYLCV) (e.g. as set forth in GenBank Accession No: LN846610.1).

According to a specific embodiment, when the plant is a Solanum tuberosum (Potato), the target gene is a gene of Phytophthora infestans (causing e.g. Late Blight) (e.g., as set forth in GenBank Accession No: AY050538.3); a gene of Erwinia spp. (causing e.g. Blackleg and Soft Rot) (e.g. as set forth in GenBank Accession No: CP001654.1); or a gene of Cyst Nematodes (e.g. Globodera pallida and G. rostochiensis) (e.g. as set forth in GenBank Accession No: KF963519.1).

According to a specific embodiment, when the plant is a Theobroma cacao (Cacao), the target is a gene of basidiomycete Moniliophthora roreri (causing e.g. Frosty Pod Rot) (e.g. as set forth in GenBank Accession No: LATX01001521.1); a gene of Moniliophthora perniciosa (causing e.g. Witches' Broom disease); or a gene of Mirids e.g. Distantiella theobroma and Sahlbergella singularis, Helopeltis spp, Monalonion species.

According to a specific embodiment, when the plant is a Vitis vinifera (Grape or Grapevine), the target gene is a gene of closterovirus GVA (causing e.g. Rugose wood disease) (e.g. as set forth in GenBank Accession No: AF007415.2); a gene of Grapevine leafroll virus (e.g. as set forth in GenBank Accession No: FJ436234.1); a gene of Grapevine fanleaf degeneration disease virus (GFLV) (e.g. as set forth in GenBank Accession No: NC_003203.1); or a gene of Grapevine fleck disease (GFkV) (e.g. as set forth in GenBank Accession No: NC_003347.1).

According to a specific embodiment, when the plant is a Zea mays (Maize also referred to as corn), the target gene is a gene of a Fall Armyworm (e.g. Spodoptera frugiperda) (e.g. as set forth in GenBank Accession No: AJ488181.3); a gene of European corn borer (e.g. as set forth in GenBank Accession No: GU329524.1); or a gene of Northern and western corn rootworms (e.g. as set forth in GenBank Accession No: NM_001039403.1).

According to a specific embodiment, when the plant is a sugarcane, the target gene a gene of an Internode Borer (e.g. Chilo Saccharifagus indicus), a gene of a Xanthomonas albileneans (causing e.g. Leaf Scald) or a gene of a Sugarcane Yellow Leaf Virus (SCYLV).

According to a specific embodiment, when the plant is a wheat, the target gene is a gene of a Puccinia striiformis (causing e.g. stripe rust) or a gene of an Aphid.

According to a specific embodiment, when the plant is a barley, the target gene is a gene of a Puccinia hordei (causing e.g. Leaf rust), a gene of Puccinia striiformis f. sp. Hordei (causing e.g. stripe rust), or a gene of an Aphid.

According to a specific embodiment, when the plant is a sunflower, the target gene is a gene of a Puccinia helianthi (causing e.g. Rust disease); a gene of Boerema macdonaldii (causing e.g. Phoma black stem); a gene of a Seed weevil (e.g. red and gray), e.g. Smicronyx fulvus (red); Smicronyx sordidus (gray); or a gene of Sclerotinia sclerotiorum (causing e.g. Sclerotinia stalk and head rot disease).

According to a specific embodiment, when the plant is a rubber plant, the target gene is a gene of a Microcyclus ulei (causing e.g. South American leaf blight (SALB)); a gene of Rigidoporus microporus (causing e.g. White root disease); a gene of Ganoderma pseudoferreum (causing e.g. Red root disease).

According to a specific embodiment, when the plant is an apple plant, the target gene is a gene of Neonectria ditissima (causing e.g. Apple Canker), a gene of Podosphaera leucotricha (causing e.g. Apple Powdery Mildew), or a gene of Venturia inaequalis (causing e.g. Apple Scab).

According to one embodiment, the plants generated by the present method are more resistant or tolerant to pathogens by at least about 10%, 20%, 30%, 40%, 50%, 60%, 70%, 80%, 90%, 95% or 100% as compared to plants not generated by the present methods (i.e. as compared to wild type plants).

Any method known in the art for assessing tolerance or resistance to a pathogen of a plant may be used in accordance with the present invention. Exemplary methods include, but are not limited to, reducing MYB46 expression in Arabidopsis which results in enhanced resistance to Botrytis cinerea as described in Ramírez V1, García-Andrade J, Vera P., Plant Signal Behav. 2011 June; 6(6):911-3. Epub 2011 Jun. 1; or downregulation of HCT in alfalfa promotes activation of defense response in the plant as described in Gallego-Giraldo L. et al. New Phytologist (2011) 190: 627-639 doi: 10.1111/j.1469-8137.2010.03621.x), both incorporated herein by reference.

According to one embodiment, there is provided a method of generating a herbicide resistant plant, the method comprising: (a) breeding the plant of some embodiments of the invention, and (b) selecting for progeny plants that are herbicide resistant.

According to one embodiment, the herbicides target pathways that reside within plastids (e.g. within the chloroplast).

Thus to generate herbicide resistant plants, the silencing insertion is designed to target a gene such as the chloroplast gene psbA (which codes for the photosynthetic quinone-binding membrane protein Q_{B}, the target of the herbicide atrazine) or the gene for EPSP synthase (a nuclear protein, however, its overexpression or accumulation in the chloroplast enables plant resistance to the herbicide glyphosate as it increases the rate of transcription of EPSPs as well as by a reduced turnover of the enzyme).

According to one embodiment, the virus targeted by the invention is an arbovirus (e.g. Vesicular stomatitis Indiana virus-VSV). According to one embodiment, the target gene is a VSV gene, e.g. G protein (G), large protein (L), phosphoprotein, matrix protein (M) or nucleoprotein.

According to one embodiment, eukaryotic cell is human and the target gene is gag and/or vif (i.e. conserved sequences in HIV-1); P protein (i.e. an essential subunit of the viral RNA-dependent RNA polymerase in RSV); P mRNA (i.e. in PIV); core, NS3, NS4B and NS5B (i.e. in HCV); VAMP-associated protein (hVAP-A), La antigen and polypyrimidine tract binding protein (PTB) (i.e. for HCV).

According to a specific embodiment, when the eukaryotic cell is human, the target gene is a gene of a pathogen causing Malaria, a gene of HIV virus (e.g. as set forth in GenBank Accession No: NC_001802.1); a gene of HCV virus (e.g. as set forth in GenBank Accession No: NC_004102.1); or a gene of Parasitic worms (e.g. as set forth in GenBank Accession No: XM_003371604.1).

According to a specific embodiment, when the eukaryotic cell is human, the target gene is a gene related to a cancerous disease (e.g. Homo sapiens mRNA for ber/abl e8a2 fusion protein, as set forth in GenBank Accession No: AB069693.1) or a gene related to a myelodysplastic syndrome (MDS) or to vascular diseases (e.g. Human heparin-binding vascular endothelial growth factor (VEGF) mRNA, as set forth in GenBank Accession No: M32977.1)

According to a specific embodiment, when the eukaryotic cell is a cattle cell, the target gene is a gene of Infectious bovine rhinotracheitis virus (e.g. as set forth in GenBank Accession No: AJ004801.1), a type 1 bovine herpesvirus (BHV1), causing e.g. BRD (Bovine Respiratory Disease complex); a gene of Bluetongue disease (BTV virus) (e.g. as set forth in GenBank Accession No: KP821170.1); a gene of Bovine Virus Diarrhhoea (BVD) (e.g. as set forth in GenBank Accession No: NC_001461.1); a gene of picornavirus (e.g. as set forth in GenBank Accession No: NC_004004.1), causing e.g. Foot & Mouth disease; a gene of Parainfluenza virus type 3 (PI3) (e.g. as set forth in GenBank Accession No: NC_028362.1), causing e.g. BRD; or a gene of Mycobacterium bovis (M. bovis) (e.g. as set forth in GenBank Accession No: NC_037343.1), causing e.g. Bovine Tuberculosis (bTB).

According to a specific embodiment, when the eukaryotic cell is a sheep cell, the target gene is a gene of a pathogen causing Tapeworms disease (E. granulosus life cycle, Echinococcus granulosus, Taenia ovis, Taenia hydatigena, Moniezia species) (e.g. as set forth in GenBank Accession No: AJ012663.1); a gene of a pathogen causing Flatworms disease (Fasciola hepatica, Fasciola gigantica, Fascioloides magna, Dicrocoelium dendriticum, Schistosoma bovis) (e.g. as set forth in GenBank Accession No: AY644459.1); a gene of a pathogen causing Bluetongue disease (BTV virus, e.g. as set forth in GenBank Accession No: KP821170.1); or a gene of a pathogen causing Roundworms disease (Parasitic bronchitis, also known as ““hoose””, Elaeophora schneideri, Haemonchus contortus, Trichostrongylus species, Teladorsagia circumcincta, Cooperia species, Nematodirus species, Dictyocaulus filaria, Protostrongylus refescens, Muellerius capillaris, Oesophagostomum species, Neostrongylus linearis, Chabertia ovina, Trichuris ovis) (e.g. as set forth in GenBank Accession No: NC_003283.11).

According to a specific embodiment, when the eukaryotic cell is a pig cell, the target gene is a gene of African swine fever virus (ASFV) (causing e.g. African Swine Fever) (e.g. as set forth in GenBank Accession No: NC_001659.2); a gene of Classical swine fever virus (causing e.g. Classical Swine Fever) (e.g. as set forth in GenBank Accession No: NC_002657.1); or a gene of a picornavirus (causing e.g. Foot & Mouth disease) (e.g. as set forth in GenBank Accession No: NC_004004.1).

According to a specific embodiment, when the eukaryotic cell is a chicken cell, the target gene is a gene of Bird flu (or Avian influenza), a gene of a variant of avian paramyxovirus 1 (APMV-1) (causing e.g. Newcastle disease), or a gene of a pathogen causing Marek's disease.

According to a specific embodiment, when the eukaryotic cell is a tadpole shrimp cell, the target gene is a gene of White Spot Syndrome Virus (WSSV), a gene of Yellow Head Virus (YHV), or a gene of Taura Syndrome Virus (TSV).

According to a specific embodiment, when the eukaryotic cell is a salmon cell, the target gene is a gene of Infectious Salmon Anaemia (ISA), a gene of Infectious Hematopoietic Necrosis (IHN), or a gene of Sea lice (e.g. ectoparasitic copepods of the genera Lepeophtheirus and Caligus).

The invention also provides methods of treating or preventing the diseases listed above, comprising administering a silencing insertion or cell of the invention. The invention also provides silencing insertions and cells of the invention for use in treating or preventing the diseases listed above.

According to one embodiment, the animals generated by the present method are more resistant or tolerant to pathogens by at least about 10%, 20%, 30%, 40%, 50%, 60%, 70%, 80%, 90%, 95% or 100% as compared to animals not generated by the present methods (i.e. as compared to wild type animals).

### Alternative Aspects

In alternative aspects of any embodiment of the invention, the silencing insertion targets the same gene as the endogenous silencing sequence, but at a different sequence location in the target gene. Accordingly, in such alternative aspects, the invention provides a method of reducing the expression of a target gene comprising inserting a silencing insertion into the genome of a eukaryotic cell,
wherein the silencing insertion comprises a promoter or a part thereof, a sequence encoding a silencing RNA that silences the expression of the target gene by targeting a first target sequence, and a terminator or a part thereof,
wherein the silencing insertion has over 95% sequence identity across its length to an endogenous silencing sequence in the eukaryotic cell that comprises a promoter, a sequence encoding a silencing RNA and a terminator, and
wherein the silencing RNA encoded by the endogenous silencing sequence silences the expression of the target gene by targeting a second target sequence that is different from the first target sequence.

In these alternative aspects, the invention also provides a eukaryotic cell comprising in its genome:
a silencing insertion comprising a promoter or a part thereof, a sequence encoding a silencing RNA that silences the expression of a target gene by targeting a first target sequence, and a terminator or a part thereof,
an endogenous genome sequence that has been separated by the insertion, and
an endogenous silencing sequence comprising a promoter, a sequence encoding a silencing RNA and a terminator,
wherein the silencing insertion and the endogenous silencing sequence have over 95% sequence identity across the length of the insertion, and
wherein the silencing RNA encoded by the endogenous silencing sequence silences the expression of the target gene by targeting a second target sequence that is different from the first target sequence.

In these alternative aspects, the invention also provides an isolated silencing construct comprising a silencing insertion that comprises comprising a promoter or a part thereof, a sequence encoding a silencing RNA that silences the expression of a target gene in a eukaryotic cell, and a terminator or a part thereof,
wherein the silencing insertion has over 95% sequence identity across its length to an endogenous silencing sequence in the eukaryotic cell that comprises a promoter, a sequence encoding a silencing RNA and a terminator, and
wherein the silencing RNA encoded by the endogenous silencing sequence silences the expression of the target gene by targeting a second target sequence that is different from the first target sequence.

### GENERAL

It is to be understood that different applications of the disclosed products and methods may be tailored to the specific needs in the art. It is also to be understood that the terminology used herein is for the purpose of describing particular embodiments of the invention only, and is not intended to be limiting.

In addition, as used in this specification and the appended claims, the singular forms “a”, “an”, and “the” include plural referents unless the content clearly dictates otherwise. Thus, for example, reference to “a polypeptide” includes “polypeptides”, and the like.

Unless specifically prohibited, the steps of a method disclosed herein may be performed in any appropriate order and the order in which the steps are listed should not be considered limiting.

All publications, patents and patent applications cited herein are hereby incorporated by reference in their entirety.

### EXAMPLES

### Example 1—Design Considerations for a Silencing Insertion According to the Invention

The following describes various considerations that may be taken into account when designing a silencing insertion to be introduced into the genome according to embodiments of the method of the present invention.

The silencing insertion sequence which is introduced into a eukaryotic cell according to some embodiments of the invention comprises a promoter, a sequence encoding a silencing RNA and a terminator. This sequence encoding the silencing RNA has over 95% sequence identity across its length to an endogenous silencing sequence which exists in the eukaryotic cell (e.g. comprising a promoter, a gene encoding a silencing ncRNA and a terminator). There may be various considerations for selecting the endogenous silencing sequence (the “scaffold”) on which the silencing insertion is based, including, but not limited to, the following:
1. Expression profile—Public or proprietary ncRNA datasets (e.g. small RNA sequencing, RNA sequencing, genomic sequences, microarrays etc.) are searched so as to filter (i.e. elect) relevant ncRNA that match input criteria (e.g. a desired spatio-temporal expression patterns). For example, suitable endogenous sequences may be selected based on whether the silencing RNA encoded by the silencing insertion is meant to be expressed ubiquitously (e.g. constitutively) or specifically (e.g. expression specific to a certain tissue, developmental stage, stress, heat/cold shock etc.). This may also be determined by the location/timing of expression of the endogenous promoter selected. Possible databases may be:
   miRbase (Kozomara, A & Griffiths-Jones, S 2011, ‘MiRBase: Integrating microRNA annotation and deep-sequencing data’, Nucleic acids research., vol. 39, no. 1, pp. D152-D157. https://doi.org/10.1093/nar/gkq1027)
   tasiRNAdb (Changqing Zhang, Guangping Li, Shinong Zhu, Shuo Zhang, Jinggui Fang, tasiRNAdb: a database of ta-siRNA regulatory pathways, Bioinformatics, Volume 30, Issue 7, 1 Apr. 2014, Pages 1045-1046, https://doi.org/10.1093/bioinformatics/btt746)
   mirEx 2.0 (Zielezinski, Andrzej et al. “mirEX 2.0—an Integrated Environment for Expression Profiling of Plant microRNAs.” BMC Plant Biology 15 (2015): 144. PMC. Web. 15 Sep. 2018).
   Plant Non-coding RNA database (Xin Yi, Zhenhai Zhang, Yi Ling, Wenying Xu, Zhen Su, PNRD: a plant non-coding RNA database, Nucleic Acids Research, Volume 43, Issue D1, 28 Jan. 2015, Pages D982-D989, https://doi.org/10.1093/nar/gku1162)
   Depending on the desired silencing using the silencing insertion, a suitable ncRNA expression is selected (e.g. low constitutive expression, highly expressed, induced in stress etc.). For high silencing activity, highly expressed ncRNA may be desired, which can be determined from high read numbers present in sRNA-Seq databases. Small RNA expression data can be aligned to a genome to establish the amount of reads that map to each genomic location (e.g. reads 19-24 bp long, are used to plot the distribution of the reads that match the corresponding endogenous sequence).
2. Silencing efficiency—The silencing efficiency of the silencing insertion may be affected by the identity of the endogenous ncRNA on which it is based (as well as by factors such as the identity of the gene targeted by the silencing insertion). Thus, based on the desired degree of silencing of the silencing insertion, the endogenous ncRNA selected will have a certain silencing activity (which causes an absence or observable reduction in the protein and/or mRNA product levels of its target gene). Silencing efficiency of ncRNA can be extracted from databases such as those noted above or empirically determined as known in the art.
3. Degree of homology between the target gene of choice (i.e. the gene to be targeted by the silencing insertion) and the ncRNA encoded by the endogenous silencing sequence on which the insertion is based—When selecting an endogenous sequence to be the basis (“scaffold”) for the silencing insertion design, it is preferable to select a sequence encoding a ncRNA that has a high degree of homology to the silencing RNA to be encoded by the silencing insertion. Thus, according to some embodiments, the endogenous sequence to be used, I.e., the endogenous “ncRNA scaffold”, is chosen once the silencing RNA to be encoded by the silencing insertion is selected. The silencing RNA to be encoded by the silencing insertion may be chosen from databases of known silencing RNAs. Preferably, the plant genome is also checked for lack of highly homologous sequences to the endogenous/exogenous target gene that could act as unintended targets (I.e. “off targets”) of the silencing RNA encoded by the silencing insertion.

Once the endogenous sequence on which the silencing insertion is to be based has been selected based on expression criteria as described above, the silencing insertion is designed. The silencing insertion comprises a promoter or a part thereof, a sequence encoding a silencing RNA precursor that is processed into a small silencing RNA (mature sRNA) that silences the expression of the first target gene, and a terminator or a part thereof. The sequence encoding the mature sRNA within the silencing insertion may be selected based on the sequence of the target gene and on sequences of silencing RNAs that are known in the art (e.g. listed in databases such as those noted above), empirically identified or identified using bioinformatic tools known in the art. Preferably, the mature sRNA perfectly matches the target's sequence. The precursor silencing RNA within the silencing insertion is therefore highly similar to the endogenous precursor silencing RNA on which it is based, with minimal changes which enable it to target the target gene of choice. Optionally, the sequence changes between the mature silencing sRNA processed from the silencing RNA encoded by the silencing insertion and the endogenous silencing RNA on which it is based depend on whether the type of silencing RNA used is affected by secondary structure. For example, when the type of silencing RNA within the silencing insertion is such that a secondary structure does not play a role in its proper biogenesis and/or function (e.g. tasiRNA) it might be sufficient for the silencing RNA within the silencing insertion to differ from its corresponding endogenous sequence only by a few modifications (e.g. by 20-30, 1-10 or 5 nucleotides). When the silencing RNA is of a type that has an essential secondary structure (i.e. the proper biogenesis and/or activity of the RNA silencing molecule is dependent on its secondary structure; for example miRNAs), the silencing RNA within the silencing insertion may differ from its corresponding endogenous sequence by a higher number of nucleotides in order to achieve the silencing specificity while maintaining secondary structure (e.g. 10-200 nucleotides, 50-150 nucleotides, more than 30 nucleotides, not exceeding 200 nucleotides, 30-200 nucleotides, 35-200 nucleotides, 35-150 nucleotides or 35-100 nucleotides).

The silencing insertion also includes promoter and terminator regions which are identical to those of the endogenous sequence on which the insertion is based.

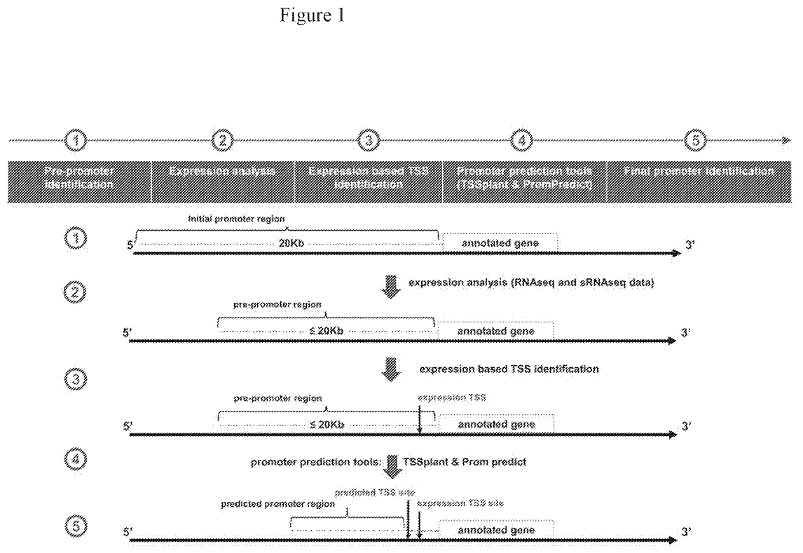

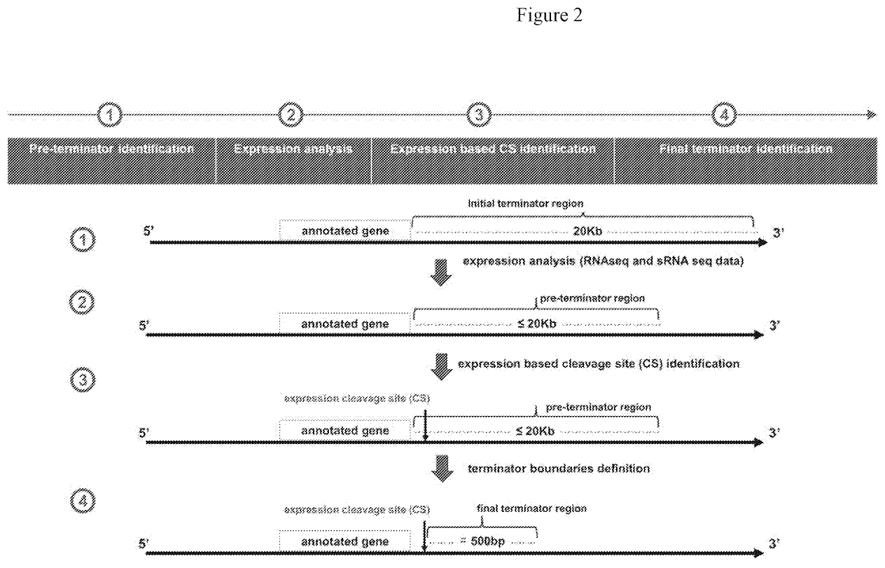

The promoter region which is included in the silencing insertion can be selected in various ways, for example:
1. Using the known promoter-Promoter databases contain annotated promoters with experimentally verified transcription start sites (TSS) and experimental evidence can further be found published literature. For example, the average promoter length in Arabidopsis thaliana is about 0.5 kb (Korkuc P, Schippers J H, Walther D. Characterization and identification of cis-regulatory elements in Arabidopsis based on single-nucleotide polymorphism information. Plant Physiol. 2014 January; 164(1):181-200. doi: 10.1104/pp. 113.229716. Epub 2013 Nov. 7. PMID: 24204023; PMCID: PMC3875800).
2. Using promoters based on bioinformatic prediction-If the genome of the species from which the promoter is to be taken is annotated, sequences can be searched against publicly available databases and bioinformatic tools can be used for identifying/predicting promoters. Known tools/databases for predicting promoters in different plant species include:
   TSSP (Tool based on PlantProm DB and Ppdb databases) (http://linux1.softberry.com/berry.phtml?topic=tssp&group-programs&subgroup-promoter)
   PlantProm DB (Database: Annotated, non-redundant collection of proximal promoter sequences for RNA polymerase II with experimentally determined transcription start site(s), TSS, from various plant species) (http://www.softberry.com/berry.phtml?topic=plantprom&group-data&subgroup=plantprom)
   Prompredict (Tool to identify promoter regions in genomic DNA sequence, based on difference in stability between neighboring regions) (http://nucleix.mbu.jisc.ernet.in/prompredict/prompredict.html).
   Ppdb (Database) (http://ppdb.agr.pifu-u.ac.jp/ppdb/cgi-bin/index.cgi)
   NSITE-PL (Tool: search for consensus patterns of plant regulatory sequences) (http://www.softberry.com/berry.phtml?topic==nsitep&group=help&subgroup=promoter)
   PLACE (Database of motifs found in plant cis-acting regulatory DNA elements) (https://www.dna.affrc.go.jp/PLACE/?action=newplace)
   PlantCARE (Database of plant cis-acting regulatory elements) (http://bioinformatics.psb.ugent.be/webtools/plantcare/html/)
   MBMEDA (Tool) (https://github.com/JmonkG/MBMEDA)
   CoVote (Common query Voting) (Tool to predict unknown core promoter of miRNA)
   Methods of MicroRNA Promoter Prediction and Transcription Factor Mediated Regulatory Network Zhao Y, Wang F, Chen S, Wan J, Wang G. Methods of MicroRNA Promoter Prediction and Transcription Factor Mediated Regulatory Network. Biomed Res Int. 2017; 2017:7049406. doi: 10.1155/2017/7049406. Epub 2017 Jun. 5. PMID: 28656148; PMCID: PMC5474535.
   AtmiRNET (Database built from NGS data) (http://AtmiRNET.itps.ncku.edu.tw/)
   PlantPAN 3.0 (Tool for for detecting transcription factor binding sites and regulatory elements) (http://plantpan.itps.ncku.edu.tw/)
   Moreover, open reading frames (ORFs) and ncRNA sequences can be searched against annotated genomes and sequences in the 5′ region of such ORFs or ncRNA can be retrieved.
   If the species genome is not annotated, then gene expression data can be used instead. Transcription start sites can be identified by applying full-length cDNA/5;-5′ESTs mapping, CAGE (Cap Analysis Gene Expression) and SAGE (Serial Analysis of Gene Expression) approaches. Since TSS selection depends on cell/tissue type, development stage and environmental conditions NGS data enables to identify promoter sequences in different contexts. RT-qPCR, Northern blot, ChIP-seq and microarray analysis can also be used to assess promoter expression profile. Promoter activity and their expression profile can also be identified by fusing sequences at the 5′ region of reporter genes (e.g: uidA, gfp, luc). When a sequence that contains a promoter is identified progressively smaller gene fragments derived from such sequence can be fused at the 5′ region of the reporter gene to identify the minimal promoter region.
   Promoter analysis in banana was demonstrated, for example in, Santos E. et al. (2016) Promoter Analysis in Banana. In: Mohandas S., Ravishankar K. (eds) Banana: Genomics and Transgenic Approaches for Genetic Improvement. Springer, Singapore. https://doi.org/10.1007/978-981-10-1585-4_11.
3. Identifying sufficient promoter fragments based on expression data—In order to identify promoter regions, we tested a bioinformatic identification pipeline in A. thaliana and M. acuminata according to the following process, which is summarised in FIGS. 1 and 2 and set out below:

### Genomes and Genomic Annotations

The genome and genomic annotation of A. thaliana were obtained from TAIR (version 10). In addition, all known miRNA precursors and mature sequences for A. thaliana were obtained from miRBase (version 22) (The microRNA Registry. Griffiths-Jones S. Nucleic Acids Res 2004 32:D109-D111). The banana genome was obtained from the banana genome hub (Musa acuminata DH Pahang v2) with its corresponding gene structure and function information in gff format. To identify phased RNAs and miRNAs, two in-house prediction pipelines were utilized.

### Small RNA-Seq Datasets

A total of 33 A. thaliana small RNA-seq sequencing samples were extracted from publicly available resources. The datasets samples from various tissues: 7 seedlings samples, 5 root samples, 2 shoot samples, 5 leaf samples, 6 inflorescence samples 3 flowers samples and 4 other different tissues. A total of 14 M. acuminata small RNA samples from several developmental stages/tissues were derived and sequenced internally-green flesh (2 samples), green peel (2 samples), rolled leaf (2 samples), top leaf (2 samples), root (2 samples), yellow peel (2 samples) and two samples from yellow flesh.

### RNA-Seq Datasets

A total of 35 A. thaliana RNA-seq sequencing samples were extracted from publicly available resources. The datasets samples from various tissues: 10 leaf samples, 8 root samples, 7 seedlings samples, 3 young seedlings samples, 2 shoot samples, 3 ovules samples and 2 silique samples. A total of 14 M. acuminata mRNA samples from several developmental stages/tissues were derived and was sequenced internally-green flesh (2 samples), green peel (2 samples), rolled leaf (2 samples), top leaf (2 samples), root (2 samples), yellow peel (2 samples) and two samples from yellow flesh.

The RNA-seq samples were subjected to quality trimming using Cutadapt. In order to identify the adapter sequences in the small RNA-seq samples, a QC analysis was performed for each sample and the adapters trimmed using Cutadapt. All the samples were then aligned to the genome of the corresponding species using STAR and the output alignment files were sorted using Samtools.

### Detection of Expression-Based Transcription Start Site and Cleavage Site

For each annotated or predicted gene, we identified the genomic coordinates of the transcription start site and cleavage site of genes based on the sRNA-seq and RNA-seq expression data. We used the small RNA-seq and RNA-seq data to record the reads that matched the genomic location of the gene and 20 Kb positions upstream and downstream to the gene. Raw read counts were normalized to RPM using the formula below:

$${RPM}_{i} = \frac{{X}_{i}}{N} \cdot {10}^{6}$$

where Xᵢ=number of reads mapping to the genomic coordinates of Xᵢ N=total number of mapped reads and 106 is the normalizing factor

Positions for which their normalized read count (RPM) is greater than or equal to a predefined threshold are considered expressed. For any given gene, its expression-based transcription start site is the most upstream position that is considered expressed given the aforementioned definition. If no expression is detected upstream to the start position of the gene then its expression-based transcription start site is, essentially, the start position of the annotated gene.

In a similar manner, the expression based cut site or end site is defined as the most downstream position to the gene for which that is expressed. If no expression is detected downstream to the gene end position, expression cut site or end site is defined as the end position of the gene.

### In-Silico Prediction of Promoter Regions

Experimental methods for promoter identification are costly, time-consuming and labor intensive. Hence, in silico methods are an attractive alternative. Promoter sequences contain multiple short DNA motifs that serve as binding sites for transcription factors (TFs) involved in specific regulation of transcription, and each promoter has a unique composition of TF binding sites. In essence, a promoter prediction algorithm identifies promoter regions based on the idea that, promoter regions are different from other genomic regions in their features (e.g. sequence, context, structure etc.). Promoters span, most commonly, a few hundred base pairs immediately upstream to the location of the transcription start site (TSS). To predict the promoter region of a gene of interest, we employed two prediction algorithms: TSSPlant and PromPredict. TSSPlant utilizes compositional and signal features of plant promoter sequences that feed an artificial neural network-based model, while PromPredict is based on the difference in DNA stability of neighboring upstream and downstream regions relative to experimentally determined TSSs. The output of each promoter prediction method is parsed using an in-house program and the most upstream position of the predicted promoter region is then recorded for each method. The final promoter region is defined as the region spanned between the most upstream position between the two locations that were recorded in the previous step and the expression-based transcription start site.

Plant terminators for RNA pol II generally require two elements downstream the Stop codon site, at the 3′UTR, for binding of a termination complex. This complex binds an AAUAAA polyadenylation (Poly(A)) motif and a U- or GC-rich sequence. The transcript will be cleaved at a site between these two elements. The processing site consists of a CA or UA sequence contained in a U-rich region and is usually 10 to 30 nt downstream the AAUAAA site in Arabidopsis and 10 to 35 nt in rice. The Poly(A) Polymerase is part of the termination complex and will add a poly-A tail at the 3′ end of the cleaved transcript. The average length of a 3′UTR is 242 bp in Arabidopsis thaliana and 469 bp in Oryza sativa.

The terminator region which is included in the silencing insertion can be selected in various ways, for example:
1. Terminator in an annotated genome-If a genome is annotated then it can be scanned for termination signals downstream a specific ORF or ncRNA sequence of interest. RNA-seq can be searched against existing databases containing authenticated Poly(A) motifs.
2. Terminator in a non-annotated genome-If a genome has not been annotated terminators can be identified by NGS. PolyA tails can be identified by subjecting RNA samples to a 3′-RACE (Rapid Amplification of cDNA Ends) assay followed by sequencing. Terminator regions are found immediately upstream of these PolyA regions. Additionally, RHAPA (RNase H alternative polyadenylation assay) can also be used for direct validation of polyadenylation sites. Terminator activity can also be identified by fusing sequences at the 3′ region of reporter genes (e.g: uidA, gfp, luc) under control of a promoter. When a sequence that contains a terminator is identified progressively smaller gene fragments derived from such sequence can be fused at the 3′ region of the reporter gene to identify the minimal terminator region.
3. Predication of poly adenylation sites—There are several available online tools/databases for predicting polyadenylation sites in different plant species:
   SignalSleuth2 (http://www.g3journal.org/lookup/suppl/doi: 10.1534/23.114.010249/-(DC1/FileS2.zip)
   PASS (Poly(A) Site Sleuth) (Tool) (https://static-content.springer.com/esm/art %3A10.1186%2F1471-2105-8-43/MediaObjects/12859_2006_1415_MOESM1_ESM.pdf)
   PASPA (Poly(A) Site Prediction in Plants and Algae) (Tool) (http://bmi.xmu.edu.cn/paspa)
   PlantAPAdb (Database for Alternative Poly(A) sites in plants)(http://bmi.xmu.edu.cn/plantAPAdb/index.php)(http://www.bmibig.cn/plantAPAdb/)

Alternatively, a terminator region of a given gene may be defined as the stretch of about 500 bp located downstream to its expression cut site or end site (transcript '3 terminus determined by the sRNA-seq and RNA-seq analysis).

### Example 2—Silencing Insertion Designs Targeting Genes in A. thaliana and in the Nematode Globodera rostochiensis

Below is a description of two silencing insertions which have been designed (also referred to herein as “GEiGS™-Insertions”). One of the silencing insertions encodes a miRNA which targets a phytoene desaturase (PDS) gene in A. thaliana and the other silencing insertion encodes a tasiRNA which targets a gene in the nematode Globodera rostochiensis.

### Computational Pipeline to Generate Silencing Insertion Designs

The non-coding RNAs (ncRNA) which are encoded by the silencing insertions described below have been designed using a computational pipeline. This pipeline applies biological metadata to find a ncRNA design that can silence the target gene of choice whilst only minimally changing the endogenous silencing gene on which the silencing insertion is based.

Briefly, the pipeline is fed input which may include: a) the target sequence to be silenced; b) the host organism to express the silencing insertion; c) the type of ncRNA to be encoded by the silencing insertion; and d) the desired expression pattern of the silencing insertion. The computational process then searches ncRNA datasets (e.g. small RNA sequencing, microarray etc.) for ncRNA that match the input criteria and have a high complementary level with the target's sequence. The sequences of the ncRNAs are then modified to perfectly match the target's sequence and the modified mature ncRNA sequences are run through an algorithm that predicts their silencing potency. The ncRNA to be included in the silencing insertion is selected out of the ncRNA that are predicted to have the highest silencing potency.

### Design of Silencing Insertion Targeting PDS3 in Arabidopsis

The miR173 from Arabidopsis thaliana (ath-miR173) has been selected as basis/scaffold for this silencing insertion. Ath-miR173 is ubiquitously highly expressed. It is listed in publications and databases, its stem loop structure is described and experimental evidence and sequence is available for mature miRNA. The ath-miR173 sequence was obtained from the miRBase database [Kozomara, A. and Griffiths-Jones, S., Nucleic Acids Res (2014) 42: D68, ÄiD73]. The sequence of the wild-type Ath-miR173 is:

(SEQ ID NO: 1)
5′-taagtactttcgcttgcagagagaaatcacagtggtcaaaaa
agttgtagttttcttaaagtctctttcctctgtgattctctgtgt
aagcgaaagagcttg-3′

The sequence of the ncRNA within the silencing insertion (based on ath-miR173) is:

(SEQ ID NO: 2)
5′taagtactttgacaatccagccaatccagcagtggtcaaaaaa
gttgtagttttcttaaagtctctttcctctgctgattggcaggat
tgtcaaagagcttg-3′

The sequence of the siRNA encoded by the silencing insertion is:
5′-tgacaatccagccaatccagc-3′ (21 nt) (SEQ ID NO: 3)

The mature processed small RNA will include an additional “t” in the 5′ that does not match the target sequence. Hence, mature modified siRNA would be 22 nt.

The target gene (named AT4G14210.1, having Tair Accession Sequence: 2129517 and GenBank Accession NM_117498) is a phytoene desaturase (PDS) gene which is involved in pigment accumulation (and thus its silencing causes photobleaching). Its sequence is as follows:

(SEQ ID NO: 4)
5′-ATGGTTGTGTTTGGGAATGTTTCTGCGGCGAATTTGCCTTAT
CAAAACGGGTTTTTGGAGGCACTTTCATCTGGAGGTTGTGAACTA
ATGGGACATAGCTTTAGGGTTCCCACTTCTCAAGCGCTTAAGACA
AGAACAAGGAGGAGGAGTACTGCTGGTCCTTTGCAGGTAGTTTGT
GTGGATATTCCAAGGCCAGAGCTAGAGAACACTGTCAATTTCTTG
GAAGCTGCTAGTTTATCTGCATCCTTCCGTAGTGCTCCTCGTCCT
GCTAAGCCTTTGAAAGTTGTAATTGCTGGTGCTGGATTGGCTGGA
TTGTCAACTGCAAAGTACCTGGCTGATGCAGGCCACAAACCTCTG
TTGCTTGAAGCAAGAGATGTTCTTGGTGGAAAGATAGCTGCATGG
AAGGATGAAGATGGGGACTGGTATGAGACTGGTTTACATATTTTC
TTCGGTGCTTATCCGAATGTGCAGAATTTATTTGGAGAACTTGGG
ATCAATGATCGGTTGCAGTGGAAGGAACACTCCATGATTTTTGCT
ATGCCAAGTAAACCTGGAGAATTTAGTAGATTTGACTTCCCAGAT
GTCCTACCAGCACCCTTAAATGGTATTTGGGCTATTTTGCGGAAC
AACGAGATGCTGACATGGCCAGAGAAAATAAAGTTTGCTATTGGA
CTTTTGCCAGCCATGGTCGGCGGTCAGGCTTATGTTGAGGCCCAA
GATGGTTTATCAGTCAAAGAATGGATGGAAAAGCAGGGAGTACCT
GAGCGCGTGACCGACGAGGTGTTTATTGCCATGTCAAAGGCGCTA
AACTTTATAAACCCTGATGAACTGTCAATGCAATGCATTTTGATA
GCTTTGAACCGGTTTCTTCAGGAAAAACATGGTTCCAAGATGGCA
TTCTTGGATGGTAATCCTCCGGAAAGGCTTTGTATGCCAGTAGTG
GATCATATTCGATCACTAGGTGGGGAAGTGCAACTTAATTCTAGG
ATAAAGAAAATTGAGCTCAATGACGATGGCACGGTTAAGAGTTTC
TTACTCACTAATGGAAGCACTGTCGAAGGAGACGCTTATGTGTTT
GCCGCTCCAGTCGATATCCTGAAGCTCCTTTTACCAGATCCCTGG
AAAGAAATACCGTACTTCAAGAAATTGGATAAATTAGTTGGAGTA
CCAGTTATTAATGTTCATATATGGTTTGATCGAAAACTGAAGAAC
ACATATGATCACCTACTCTTTAGCAGAAGTAACCTTCTGAGCGTG
TATGCCGACATGTCCTTAACTTGTAAGGAATATTACGATCCTAAC
CGGTCAATGCTGGAGCTAGTATTTGCACCAGCAGAGGAATGGATA
TCACGGACTGATTCTGACATCATAGATGCAACAATGAAAGAACTT
GAGAAACTCTTCCCTGATGAAATCTCAGCTGACCAAAGCAAAGCT
AAAATTCTGAAGTACCATGTCGTTAAGACTCCAAGATCTGTGTAC
AAGACCATCCCAAACTGTGAACCATGTCGTCCTCTACAAAGATCA
CCTATTGAAGGATTCTACTTAGCTGGAGATTACACAAAACAGAAG
TACTTAGCTTCCATGGAAGGCGCTGTCCTCTCTGGCAAATTCTGC
TCTCAGTCTATTGTTCAGGATTACGAGCTACTGGCTGCGTCTGGA
CCAAGAAAGTTGTCGGAGGCAACAGTATCATCATCATGA-3′

The sequence of the siRNA target site is as follows: 5′-GCTGGATTGGCTGGATTGTCA-3′ (SEQ ID NO: 5)

The estimation of miR173 promoter, terminator and gene regions have been determined as follows and used in the design of the silencing insertion:
Promoter—AtmiRNET database built from NGS data was used to estimate the promoter region. Two transcription sites were predicted 64 bp and 104 bp upstream from the miR173 ncRNA transcript sequence. The TSSP tool (based on PlantProm DB and Ppdb databases) predicted the presence of a promoter and TATA box less than 100 nt upstream of the miR173 ncRNA transcript sequence. Taking these predictions into consideration and the average promoter length in Arabidopsis thaliana (500 bp), 500 bp immediately upstream of the miR173 ncRNA were selected as promoter region.
Terminator—For the terminator region the average 3′UTR length in Arabidopsis thaliana was considered (242 bp) and 400 bp immediately downstream of the miR173 ncRNA transcript sequence were selected to act as terminator.
Gene—The miR173 sequence was obtained from the miRBase database.

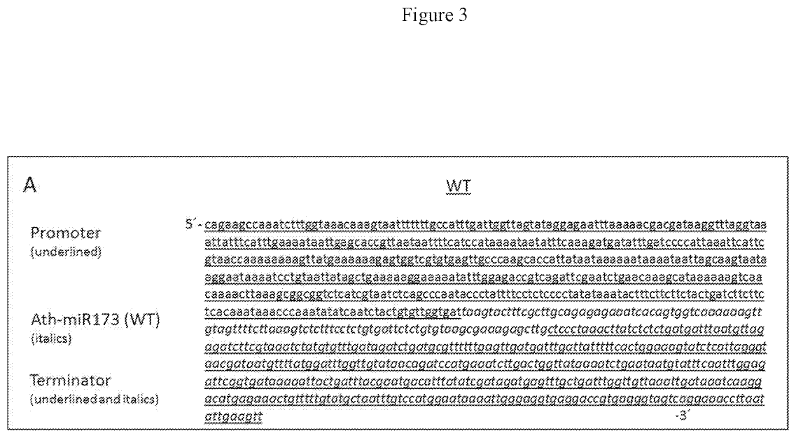

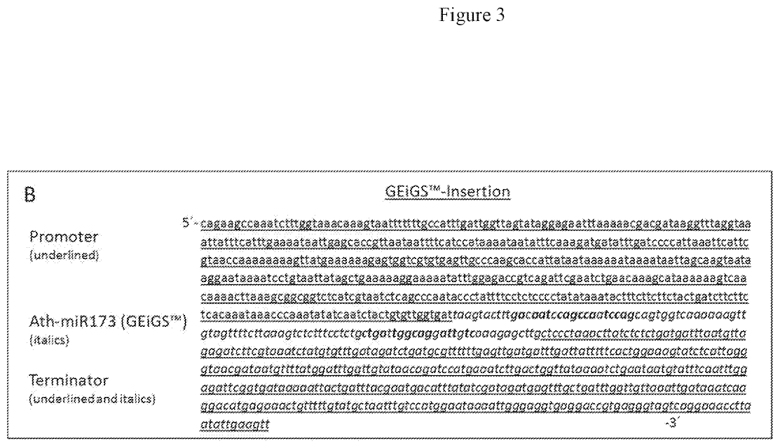

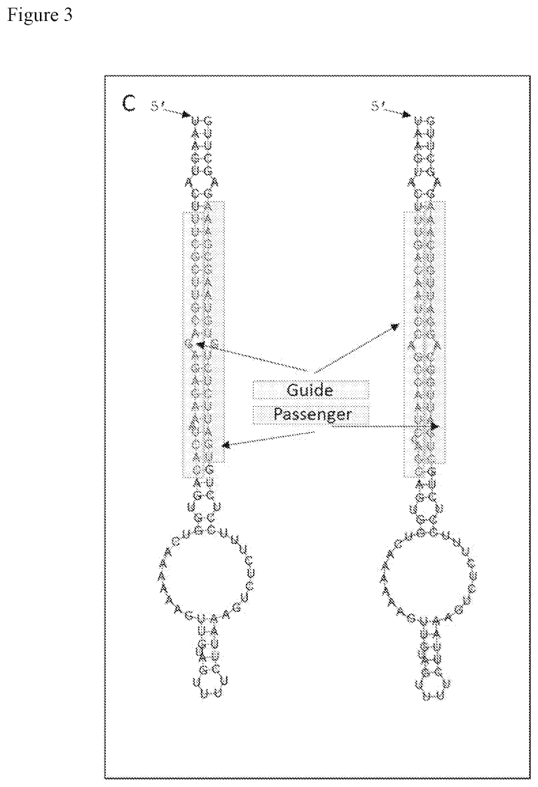

The fully designed silencing insertion vs the wild-type miR173 sequence are depicted in FIGS. 3A-C.

Design of Silencing Insertion Targeting Ribosomal Protein 3a in Globodera rostochiensis

As basis/scaffold for this silencing insertion gene, the Tas3a from Arabidopsis thaliana (ath-Tas3a) was selected, which encodes a trans-acting-siRNA-producing (TAS) molecule. The sequence of the silencing insertion was designed to include the Tas3a sequence with specific nucleotide changes such that it gives rise to long dsRNA and small secondary tasiRNA that would target and silence an essential gene in the nematode Globodera rostochiensis. At3g17185 has been shown to be a ta-siRNA-generating locus. It is listed in publications and databases and experimental evidence and sequence is available for siRNA. There is no secondary structure involved in the silencing caused by dsRNA. The tasiRNA precursor and mature sequences were obtained from the tasiRNAdb database [Zhang, C. et al, Bioinformatics (2014) 30: 1045,Äì1046].

WT ath-Tas3a ncRNA Sequence:

(SEQ ID NO: 6)
5′-ATCCCACCGTTTCTTAAGACTCTCTCTCTTTCTGTTTTCTAT
TTCTCTCTCTCTCAAATGAAAGAGAGAGAAGAGCTCCCATGGATG
AAATTAGCGAGACCGAAGTTTCTCCAAGGTGATATGTCTATCTGT
ATATGTGATACGAAGAGTTAGGGTTTTGTCATTTCGAAGTCAATT
TTTGTTTGTTTGTCAATAATGATATCTGAATGATGAAGAACACGT
AACTAAGATATGTTACTGAACTATATAATACATATGTGTGTTTTT
CTGTATCTATTTCTATATATATGTAGATGTAGTGTAAGTCTGTTA
TATAGACATTATTCATGTGTACATGCATTATACCAACATAAATTT
GTATCAATACTACTTTTGATTTACGATGATGGATGTTCTTAGATA
TCTTCATACGTTTGTTTCCACATGTATTTACAACTACATATATAT
TTGGAATCACATATATACTTGATTATTATAGTTGTAAAGAGTAAC
AAGTTCTTTTTTCAGGCATTAAGGAAAACATAACCTCCGTGATGC
ATAGAGATTATTGGATCCGCTGTGCTGAGACATTGAGTTTTTCTT
CGGCATTCCAGTTTCAATGATAAAGCGGTGTTATCCTATCTGAGC
TTTTAGTCGGATTTTTTCTTTTCAATTATTGTGTTTTATCTAGAT
GATGCATTTCATTATTCTCTTTTTCTTGACCTTGTAAGGCCTTTT
CTTGACCTTGTAAGACCCCATCTCTTTCTAAACGTTTTATTATTT
TCTCGTTTTACAGATTCTATTCTATCTCTTCTCAATATAGAATAG
ATATCTATCTCTACCTCTAATTCGTTCGAGTCATTTTCTCCTACC
TTGTCTATCCCTCCTGAGCTAATCTCCACATATATCTTTTGTTTG
TTATTGATGTATGGTTGACATAAATTCAATAAAGAAGTTGACGTT
TTTCT-3′

GEiGS™-Insertion ath-Tas3a ncRNA Sequence:

(SEQ ID NO: 7)
5′-ATCCCACCGTTTCTTAAGACTCTCTCTCTTTCTGTTTTCTAT
TTCTCTCTCTCTCAAATGAAAGAGAGAGAAGAGCTCCCATGGATG
AAATTAGCGAGACCGAAGTTTCTCCAAGGTGATATGTCTATCTGT
ATATGTGATACGAAGAGTTAGGGTTTTGTCATTTCGAAGTCAATT
TTTGTTTGTTTGTCAATAATGATATCTGAATGATGAAGAACACGT
AACTAAGATATGTTACTGAACTATATAATACATATGTGTGTTTTT
CTGTATCTATTTCTATATATATGTAGATGTAGTGTAAGTCTGTTA
TATAGACATTATTCATGTGTACATGCATTATACCAACATAAATTT
GTATCAATACTACTTTTGATTTACGATGATGGATGTTCTTAGATA
TCTTCATACGTTTGTTTCCACATGTATTTACAACTACATATATAT
TTGGAATCACATATATACTTGATTATTATAGTTGTAAAGAGTAAC
AAGTTCTTTTTTCAGGCATTAAGGAAAACATAACCTCCGTGATGC
ATAGAGATTATTGGATCCGCTGTGCTGAGACATTGAGTTTTTCTT
CGGCATTCCAGTTTCAATGATAAAGCGGTGTTATCCTATCTGAGC
TTTTAGTCGGATTTTTTCTTTTCAATTATTGTGTTTTATCTAGAT
GATGCATTTCATTATTCTCTTTTTCTTGACCTTGTAAGGCCTTTT
CTTGACCTTGTAAGACCCCATCTCTTTCTAAACGTTTTATTATTT
TCTCGTTTTACAGATTCTATTCTATCTCTTCTCAATATAGAATAG
ATATCGGCTTCTTCAGCACCTTCACCTTACGAATTTTCTCCTACC
TTGTCTATCCCTCCTGAGCTAATCTCCACATATATCTTTTGTTTG
TTATTGATGTATGGTTGACATAAATTCAATAAAGAAGTTGACGTT
TTTCT-3′

The Ribosomal protein 3a target gene of Globodera rostochiensis was chosen based on previous publications that discussed negative effects in a nematode when genes were targeted using an RNAi technology. Since this gene was identified in a different strain of nematodes its homologue was identified through a BLAST search in the Globodera rostochiensis publicly available database (https://parasite.wormbase.org/Globodera_rostochiensis_prjeb13504/Info/Index/), using the chosen gene as query.

| Target | | | | | | Homologue in |
|---|---|---|---|---|---|---|
| gene | Gene | | Host | | | Globodera |
| Nematode | symbol | Species | plant | Phenotype | Reference | rostochiensis |
| Ribosomal | CB379877 | H. glycines | Soybean | 87% reduction | Klink et | GROS_g04462 |
| protein 3a | | | | in number of | al., 2009 | |
| | | | | female cysts | | |

### Nematode Target Gene Sequence (Target Sequence is Shown Underlined):

(SEQ ID NO: 8)
5′-ATGGCAGTCGGAAAGAATAAGAAAATGGGCAAAAAGGGAGCCAA
GAAGAAGGCTGTCGATCCGTTCACACGCAAAGAATGGTACGACATCA
AAGCGCCGGCGATGTTCACACATCGAAACGTCGGCAAAACGTTGGTC
AACCGTACTCAGGGAACGCGCATTTCGAGCGACTTTCTAAAAGGCCG
CGTTTACGAAGTGTCACTGGGTGACCTTAACAGCACTGACGCCGACT
TTCGAAAGTTCCGCCTGATCTGTGAAGAGGTACAGGGCAAGATTTGC
CTGACCAACTTTCACGGAATGTCGTTCACTCGGGACAAACTGTGCTC
TATTGTCAAGAAGTGGCACACGCTCATTGAGGCGAATGTGGCAGTGA
AGACTACCGACGGTTTCATGCTCCGACTCTTTTGTATCGGCTTTACC
AAGCGAAATGCCAATCAAATTAAGAAGACGAGCTATGCAAAAGCCTC
TCAGGTGCGGATGATTCGTGCCAAAATGGTGGAGATCATGCAGAAAG
AGGTCTCTTCCGGCGATCTGAAGGAAGTAGTCAACAAGCTGATCCCG
GATTCGATCGGCAAAGACATAGAGAAGGCGTGCTCCTTCTACTACCC
TCTGCAGGACGTTTACATTCGTAAGGTGAAGGTGCTGAAGAAGCCGA
AGTTCGAGCTGGGCAAACTATTGGAGATGCATGGGGAGGGTGCCGGA
ACGGTCGCTACGATTACGACGGCCGCCGGTGAAAAAATTGAGAGCCG
TCCGGATGCGTACGAACCGCCTGTTCAACAGAGCGTTTGA-3′

The siRNA Target Site Sequence within the Target Gene:

(SEQ ID NO: 9)
5′-ATTCGTAAGGTGAAGGTGCTGAAGAAGCCG-3′

### The Homologous Region in the Silencing Insertion Design:

(SEQ ID NO: 10)
5′-CGGCTTCTTCAGCACCTTCACCTTACGAAT-3′

Following is the siRNA sequence encoded within the silencing insertion which indicates the region of homology to the target gene. The length of the designed siRNA is 30 nt to facilitate generation of siRNA in nematodes:

(SEQ ID NO: 10)
5′-cggcttcttcagcaccttcaccttacgaat-3′

The estimation of Tas3a promoter, terminator and gene regions have been determined as follows and used in the design of the silencing insertion:
Promoter—Here, not only the average promoter length in Arabidopsis thaliana (500 bp) was considered but also the Prompredict tool was applied. This tool identifies promoter regions in genomic DNA sequence based on difference in stability between neighboring regions. 1200 bp immediately upstream of Tas3a ncRNA transcript sequence were then selected as promoter.
Terminator—PlantAPAdb database was used for estimating the terminator length. This database is derived from a large volume of data obtained 3′-seq. It predicted the presence of a poly(A) site approximately 300 bp downstream of the annotated Tas3a ncRNA transcript sequence. Taking this into consideration 500 bp were selected to act as terminator.
Gene—The Tas3 sequence was obtained from the tasiRNAdb database.

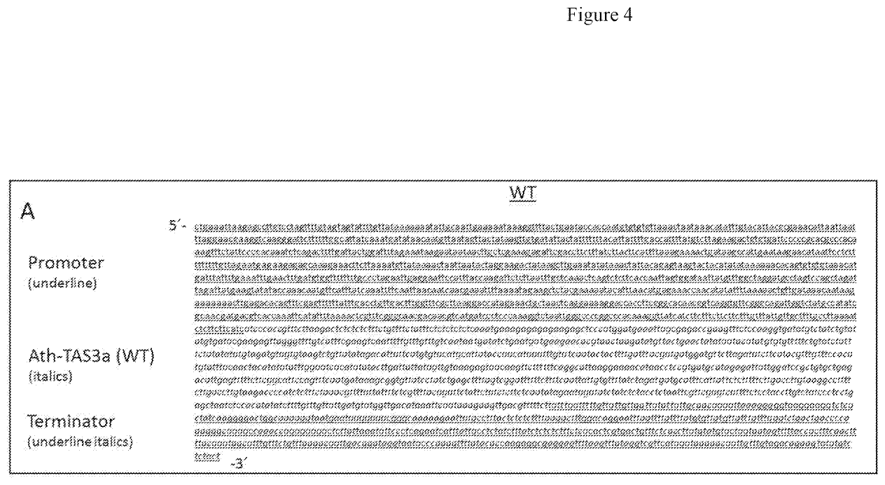

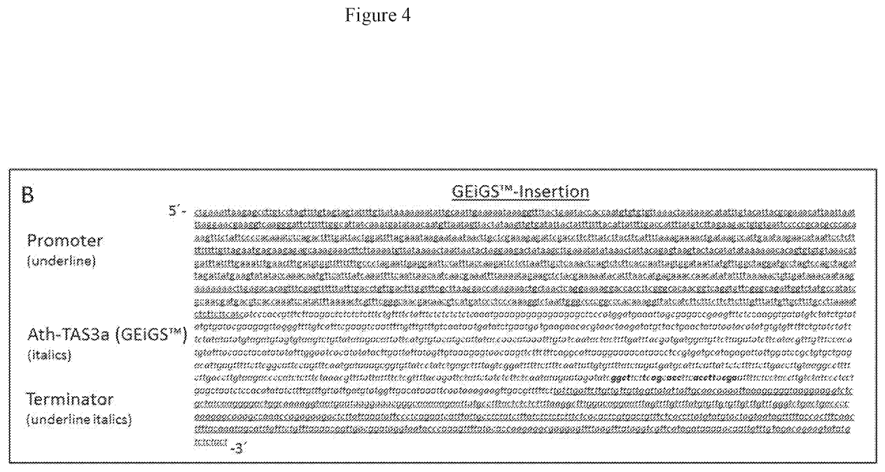

The fully designed silencing insertion vs the wild-type miR173 sequence are depicted in FIGS. 4A-B.

### Synthesis and Transformation of the Designed Insertions

The silencing insertion expression cassette is introduced into the cells as linear DNA by DNA bombardment and randomly integrated. The silencing insertion expression cassette is generated by chemical synthesis and amplified as a whole by high-fidelity PCR. Alternatively, the silencing insertion cassette is generated by overlapping PCR reactions to introduce the relevant modifications into the sequence on which the insertion is based using genomic DNA as template (FIG. 5). After PCR amplification and before restriction digestion, which is required to generate sticky ends to enhance integration, DNA might need to be concentrated by using centrifugal filters available as commercial kits.

There are alternative configurations possible for the 5′ and 3′ termini of the linear DNA molecule:
Blunt-end molecules can be produced using standard oligonucleotide primers and no further processing. Once PCR amplified, the GEiGS™-Insertion cassettes are purified with clean-up concentrator commercial kits and yield and integrity are assessed through gel analysis and fluorometric quantitation.
Sticky-end molecules can be produced using oligonucleotide primers which contain different restriction sites at their 5′ ends. Restriction digestion of PCR products with the corresponding high-fidelity restriction enzymes produces sticky ends. Once restricted, GEiGS™-Insertion cassettes are purified with clean-up concentrator commercial kits. These kits allow to clean up DNA from enzymes and primers. Yield and integrity are assessed through gel analysis and fluorometric quantitation.
Chemically stabilised molecules can be produced using oligonucleotides which contain stabilising 5′-phosphorothioate linkages to reduce exonuclease degradation. Once PCR amplified, the GEiGS™-Insertion cassettes are purified with clean-up concentrator commercial kits and yield and integrity are assessed through gel analysis and fluorometric quantitation.

### Primers for Blunt-End GEiGS™ Cassette Production

Primers for PCR Amplification of Whole ath-miR173 GEiGS™-Insertion Example Cassette:

Primer Fw:
(SEQ ID NO: 11)
5′-cagaagccaaatctttggtaaacaaagtaatttttttg-3′
Primer Rev:
(SEQ ID NO: 12)
5′-aacttcaatattaaggtttcctgactaccctcac-3′

Primers for PCR Amplification of Whole ath-Tas3a GEiGS™-Insertion Example Cassette:

Primer Fw:
(SEQ ID NO: 13)
5′-atgaaattaagagccttgtcctagttttgtagtag-3′
Primer Fw:
(SEQ ID NO: 14)
5′-agtagagacatatacttctgtctacaaacaattgtttttatc-3′

### Primers for Sticky-End GEiGS™ Cassette Production

Primers for PCR Amplification of Whole ath-miR173 GEiGS™-Insertion Example Cassette:

Primer Fw:
(SEQ ID NO: 15)
5′-nnxxxxxxcagaagccaaatctttggtaaacaaagtaa
tttttttg-3′
Primer Rev:
(SEQ ID NO: 16)
5′-nnxxxxxxaacttcaatattaaggtttcctgactaccc
tcac-3′

Primers for PCR Amplification of Whole ath-Tas3a GEiGS™-Insertion Example Cassette:

Primer Fw:
(SEQ ID NO: 17)
5′-nnxxxxxxatgaaattaagagccttgtcctagttttgtagtag-3′
Primer Rev:
(SEQ ID NO: 18)
5′-nnxxxxxxagtagagacatatacttctgtctacaaacaattgtttt
tatc-3′

Where:
n: any nucleotide
xxxxxx: restriction site for high-fidelity enzyme.

### Primers for Chemically Stabilised GEiGS™ Cassette Production

Primers for PCR Amplification of Whole ath-miR173 GEiGS™-Insertion Example Cassette:

Primer Fw:
(SEQ ID NO: 19)
5′-c*agaagccaaatctttggtaaacaaagtaatttttttg-3′
Primer Rev:
(SEQ ID NO: 20)
5′-a*acttcaatattaaggtttcctgactaccctcac-3′

Primers for PCR Amplification of Whole ath-Tas3a GEiGS™-Insertion Example Cassette:

Primer Fw:
(SEQ ID NO: 21)
5′-a*tgaaattaagagccttgtcctagttttgtagtag-3′
Primer Rev:
(SEQ ID NO: 22)
5′-a*gtagagacatatacttctgtctacaaacaattgtttttatc-3′

Where: * represents a phosphorothioate linkage

Linear silencing insertion expression cassettes are introduced to Arabidopsis root tissue using particle bombardment protocols (Sawasaki et al., 1994, Ruf et al., 2019). Briefly, Arabidopsis root sections are cultured on callus-inducing medium for 48 hours, then microcalli are bombarded with the GEiGS™-Insertion cassette. After 48 hours recovery on callus-inducing media, microcalli are transferred to shoot-inducing media containing an appropriate selective agent. Transformed plants can then be regenerated and genotype as detailed in the following examples (References: Ruf, S., Forner, J., Hasse, C., Kroop, X., Seeger, S., Schollbach, L., Bock, R. (2019). High-efficiency generation of fertile transplastomic Arabidopsis plants. Nature Plants, 5(3), 282-289. https://doi.org/10.1038/s41477-019-0359-2; Sawasaki, T., Seki, M., Anzai, H., Irifune, K., & Morikawa, H. (1994). Stable transformation of Arabidopsis with the bar gene using particle bombardment. Transgenic Research, 3, 279-286).

### Example 3—Methods for Selection of Cells Comprising a Silencing Insertion

Below is a description of methods for selection of cells comprising a silencing insertion using dominant cisgenic selection.

### Rationale for Dominant Cisgenic Selection

A dominant allele is a specific gene variant that results in a phenotype that prevails even in the presence of other recessive alleles for the same gene. While the phenotypic effect of recessive genes becomes evident only under homozygous condition, the effects for dominant alleles are seen under both homozygous and heterozygous conditions. Dominant selection markers that arise through a gain-of-function mutation are particularly useful for plant transformation and genomic editing. This mutated allele will be dominant over the susceptible non-mutated allele/s and the resistant phenotype will prevail even under heterozygous conditions. In some cases, as seen for the acetolactate synthase (ALS) gene, certain single point mutations are enough to produce a dominant herbicide-resistant phenotype. Strong dominant markers increase transformation efficiency since they can be selected for at low copy number.

### Herbicide Selection Using Naturally-Occurring Mutations

One such selection marker can be achieved by the alteration of the acetolactate synthase (ALS) gene. The nuclear ALS gene codes for an enzyme involved in the first step of biosynthesis of branched chain amino acids. Herbicides that inhibit the ALS enzyme interfere with branched amino acid synthesis, resulting in plant death. There are 5 main families of such herbicides, namely triazolopyrimidines (TP), sulfonylureas (SU), pyrimidinylthiobenzoates (PTB), imidazolinones (IMI) and sulfonylaminocarbonyltriazolinone (SCT).

Since herbicides targeting the ALS enzyme were widely adopted multiple naturally occurring non-synonymous ALS mutations have been described in weeds resistant to ALS-inhibiting herbicides. As of 2020, there are at least 22 monocot and 48 dicot weeds naturally resistant to ALS-inhibiting herbicides. Most of these cases consist of one or more single point mutations in the ALS gene.

The first mutation was described as early as 1990 (was P197T). Since then, and given the widespread use, the high selective pressure and the single point mutation nature, the appearance of herbicide-resistant ALS mutants is a frequent occurrence. As an example, mutation in proline 197 for serine (P197S) in the ALS1 gene in Arabidopsis thaliana confers chlorsulfuron (a SU variant) resistance. Mutated ALS alleles are dominant over susceptible non-mutated alleles. The resistance phenotype prevails even under heterozygous conditions (Yu Q, Han H, Vila-Aiub M M, Powles S B. AHAS herbicide resistance endowing mutations: effect on AHAS functionality and plant growth. J Exp Bot. 2010; 61(14):3925-3934. doi:10.1093/jxb/erq205; Tranel, P., & Wright, T. (2002). Resistance of weeds to ALS-inhibiting herbicides: What have we learned? <i>Weed Science, </i> <i>50</i>(6), 700-712. doi: 10.1614/0043-1745(2002)050[0700:RROWTA]2.0.CO;2).

Other possible gene/mutations that could be used have been found in naturally resistant weeds (Gaines, T. A., and Heap, I. M. Mutations in herbicide-resistant weeds to EPSP synthase inhibitors. Online http://www.weedscience.com. Dec. 1, 2020; Takano, Hudson Kagueyama, Ovejero, Ramiro Fernando Lopez, Belchior, Gustavo Gross, Maymone, Gizella Potrich Leal, & Dayan, Franck E . . . (2021). ACCase-inhibiting herbicides: mechanism of action, resistance evolution and stewardship. Scientia Agricola, 78(1), e20190102. Epub Mar. 13, 2020.https://dx.doi.org/10.1590/1678-992x-2019-0102):
psbA glycine for serine 264 mutation (G264S) results in a modified D1 protein that confers triazine-resistance
psbA isoleucine for valine 219 mutation (1219V) results in a modified D1 protein that confers triazine-resistance
enolpyruvylshikimate-3-phosphate synthase (EPSPS) tryptophan 102 mutations confer resistance to EPSP synthase inhibitors
enolpyruvylshikimate-3-phosphate synthase (EPSPS) alanine 103 mutations confer resistance to EPSP synthase inhibitors
enolpyruvylshikimate-3-phosphate synthase (EPSPS) proline 106 mutations confer resistance to EPSP synthase inhibitors

### Cisgenic Construct Design Consideration

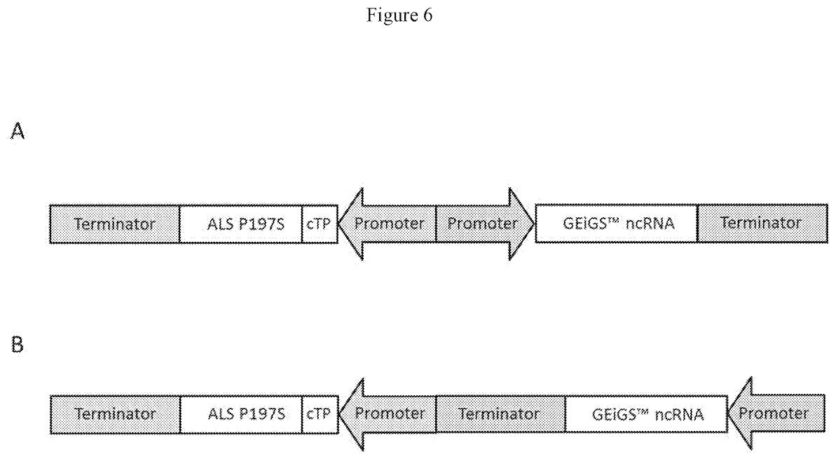

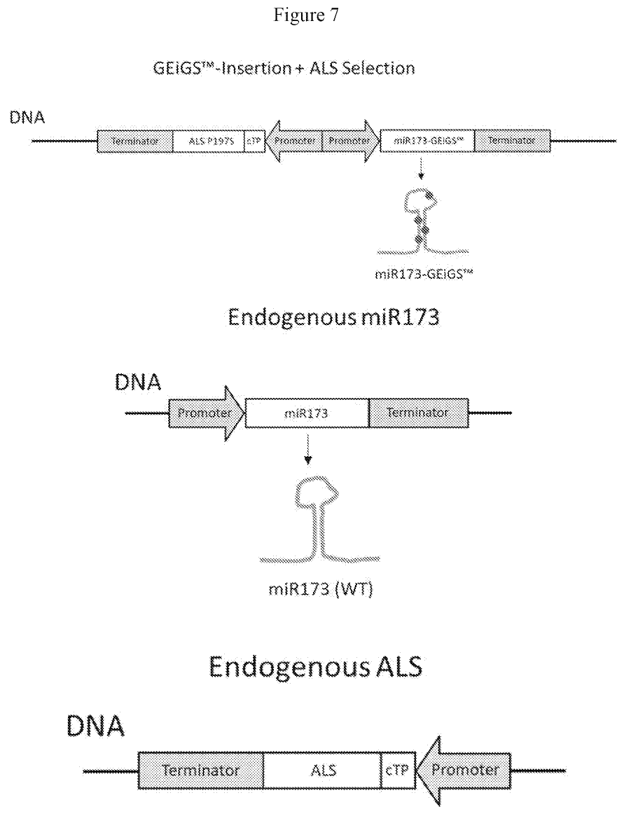

In order for a silencing-Insertion construct to be considered as cisgenic, the native promoter and terminator in the normal sense orientation have to be included. Any introns in the ncRNA sequence have to be included, if present in the native sequence (Telem R S, Wani S H, Singh N B, et al. Cisgenics—a sustainable approach for crop improvement. Curr Genomics. 2013; 14(7):468-476. doi: 10.2174/13892029113146660013). Possible ways to construct the insertion are depicted in FIG. 6. FIG. 7 depicts a possible design of a silencing insertion based on A. thaliana miR173 of Example 2 above, which has been combined with the P179S mutated form of ALS1 to generate a GEiGS™-Insertion construct suitable for selection using a herbicide.

### Estimation of Promoter, Terminator and Gene Regions for Mutated ALS Expression Cassette

Promoter: The TSSP tool (based on PlantProm DB and Ppdb databases) predicted the presence of a promoter and TATA box 120 nt upstream of the ALS open reading frame. Taking these predictions into consideration and the average promoter length in Arabidopsis thaliana (500 bp), 500 bp immediately the predicted transcription initiation site were selected as promoter region.

Terminator: PlantAPAdb database was used for estimating the terminator length. This database is derived from a large volume of data obtained 3′-seq. It predicted the presence of a poly(A) site 371 bp downstream of the termination codon. Taking this into consideration 500 bp immediately downstream the termination codon were selected to act as terminator. The promoter, terminator and gene regions for miR173 were found as described in Example 2.

### Gene (Acetolactate Synthase Small Subunit 1, Chloroplastic)

The ALS1 sequence was already annotated an available from online databases (https://www.uniprot.org/uniprot/Q9FFF4 and https://www.arabidopsis.org/servlets/TairObject?accession=locus:2114525). The open reading frame consists in 2013 nt (670 amino acids) of which 291 nt (97 amino acids) are annotated as a putative chloroplast transit peptide (cTP). The mature ALS protein is listed as 1719 nt (573 amino acids) long. The ChloroP 1.1 Server (which predicts the presence of cTP in protein sequences and the location of potential cTP cleavage sites) predicts a 157 nt cTP partly overlapping the annotated 291 nt. For mutation notation, amino acids are numbered from the beginning of the precursor ALS protein, which includes the transit peptide. The herbicide-resistant ALS version contains the single point mutation P197S in which the codon CCT is mutated to TCT.

Synthesis of Silencing-Insertion Construct with Cisgenic Selection

The silencing-Insertion constructs containing a mutated P179S ALS cassette for selection can be generated by chemical synthesis and amplified by high-fidelity PCR. Alternatively, the GEiGS™-Insertion cassette and the mutated P179S ALS cassette can be generated by overlapping PCR reactions, using genomic DNA as template, to introduce the relevant GEiGS™ modifications and single point ALS mutation using genomic DNA as template. Both constitute plasmid-free methods, as described in Example 2.

### Restriction Strategy

Primers for PCR Amplification of Whole GEiGS™-Insertion with Selection Cassette:

Primer Fw:
(SEQ ID NO: 23)
5′-nnxxxxxxcatatcaagctagattttgaattaccctatttcatcg
t-3′
Primer Rev:
(SEQ ID NO: 16)
5′-nnxxxxxxaacttcaatattaaggtttcctgactaccctcac-3′

Where:
n: any nucleotide
xxxxxx: Unique restriction site for high-fidelity enzyme. The restriction site should be unique so that there are no such sites in the GEiGS™ insertion cassette.

### Chemical Modification Strategy

Primers for PCR Amplification of Whole GEiGS™-Insertion with Selection Cassette:

Primer Fw:
(SEQ ID NO: 24)
5′-c*atatcaagctagattttgaattaccctatttcatcgt-3′
Primer Rev:
(SEQ ID NO: 20)
5′-a*acttcaatattaaggtttcctgactaccctcac-3′

Where:

A phosphorothioate bond is represented by *

### Example 4—Methods for Introducing a Silencing Insertion into Cells

Once designed, there are multiple methods for introducing the silencing insertion cassettes into cells. Common methods are biolistic bombardment, Agrobacterium-mediated transformation, and protoplast transfection. The method selected will depend on the host species and regulatory environment. Advantages of biolistic bombardment include transformation with minimal gene cassettes (no T-DNA borders) and a wider range of suitable host species. Large-scale (μg-mg) plasmid-free production of linear DNA through PCR can be used to avoid insertion of plasmid backbone sequences.

### Cloning of Silencing-Insertion Constructs

Silencing Insertion cassettes (native ncRNA promoter::modified ncRNA transcript:native ncRNA terminator) are produced by commercial gene synthesis, together with a mutated form of a native (including promoter and terminator) ALS gene cassette that will provide resistance to chlorsulfuron selection. The transcriptional units may be in the same direction or divergent, according to the genomic sequence. The construct will be provided in a plasmid backbone with antibiotic resistance, allowing selection of transformed E. coli cells containing the plasmid. The GEiGS™-Insertion plasmid can then be replicated and purified using commercial plasmid isolation kits. Below are two examples of introducing such silencing insertion into plant cells, one example using Agrobacterium in Arabidopsis and the other using biolistic bombardment in tomato.

### Agrobacterium-Mediated Transformation of Arabidopsis

The construct to be transformed includes a silencing insertion based on the miR173 sequence in Arabidopsis, in which the altered specificity of the encoded miRNA is towards the PDS3 gene. Transformation requires flowering Arabidopsis plants and Agrobacterium transformed with the insertion plasmid. The transformation can be performed essentially as described in: Clough S J & Bent, AF (1998) Floral dip: a simplified method for Agrobacterium-mediated transformation of Arabidopsis thaliana. Plant J 16:735-43. Transformed Arabidopsis seeds will be germinated on media with 100 nM chlorsulfuron as the selective agent. This will prevent growth in seedlings lacking the mutated ALS gene (and therefore lacking the silencing insertion). Plants with good growth can then be genotyped and transferred to soil for seed production.

Following germination, the transformed seedlings are genotyped to confirm the presence of the silencing insertion. Tissue from seedlings is sampled with tweezers and stored in Thermo Scientific Phire dilution buffer. PCR using Thermo Scientific Plant Phire polymerase will take place using the dilution buffer containing leaf tissue from germinated seedlings. The presence of the silencing Insertion will be confirmed using the primers below:

>At_GEiGS™_geno_F1
(SEQ ID NO: 25)
5′-acaaactacctgcaagtggtca-3′
>At_GEiGS™_geno_R1
(SEQ ID NO: 26)
5′-caagctctttatccacact-3′

Seedlings with positive genotyping PCR results (an 86 bp band is produced) are transferred to soil and grown to produce seed. To identify the location of the GEiGS™-Insertion in the Arabidopsis genome Illumina whole-genome sequencing is performed on genomic DNA. Genomic DNA is extracted using a modified CTAB method (Inglis et al., 2018). The Illumina DNA prep kit is used to prepare libraries for paired-end short-read sequencing, and libraries are sequenced using an Illumina sequencer such as Nextseq 550 (https://journals.plos.org/plosone/article?id=10.1371/journal.pone.0206085).

To identify the insertion site libraries are aligned to the Arabidopsis genome and silencing Insertion construct using bowtie2 (Langmead and Salzberg, 2012). Discordant read pairs (one read aligning to the Arabidopsis genome, one to the GEiGS™-Insertion construct) are extracted and used to design primers. These primers are then be used to amplify from the original sample genomic DNA, and sequence to identify the precise location of the insertion site (Langmead, B. & Salzberg, S. L. Fast gapped-read alignment with Bowtie 2. Nat. Methods 9, 357-359 (2012); Inglis, P. W., Marilia de Castro, R. P., Resende, L. V., & Grattapaglia, D. (2018). Fast and inexpensive protocols for consistent extraction of high-quality DNA and RNA from challenging plant and fungal samples for high-throughput SNP genotyping and sequencing applications. PLOS ONE, 13(10), 1-14. https://doi.org/10.1371/journal.pone.0206085; Lambirth, K. C., Whaley, A. M., Schlueter, J. A., Bost, K. L., & Piller, K. J. (2015). CONTRAILS: A tool for rapid identification of transgene integration sites in complex, repetitive genomes using low-coverage paired-end sequencing. Genomics Data, 6, 175-181. https://doi.org/10.1016/j.gdata.2015.09.001).

### Biolistic Bombardment-Mediated Transformation of Tomato

Biolistic bombardment allows cells to be transformed with non-transgenic DNA that does not include heterologous DNA, for example from another species. The construct to be transformed includes a silencing insertion based on the miRNA gene sly-MIR164b (GROS_g04462) in tomato, in which the altered specificity of the encoded miRNA is towards the ribosomal protein 3a gene in the nematode Globodera rostochiensis. The TSSP tool (based on PlantProm DB and Ppdb databases) predicted the presence of a promoter and TATA box with a transcription start approximately 120 nt upstream of the miR164 ncRNA transcript sequence. Taking this into consideration, 500 bp immediately upstream of the miR164 ncRNA were selected as promoter region to include in the silencing insertion. For the terminator region the average 3′UTR length in Solanum lycopersicum was considered (257 bp) and 500 bp immediately downstream of the miR164b ncRNA transcript sequence were selected to act as terminator in the silencing insertion.

Linear DNA containing only the silencing insertion (including the ALS selection cassette) is amplified from the silencing insertion plasmid using the following primers:

>TomGEiGS™_linear_F1
(SEQ ID NO: 27)
5′-A*ATTCAAAGCCAATGGAACT-3′
>TomGEiGS™_linear_R1
(SEQ ID NO: 28)
5′-A*GTACAAGATGTCAAATGCC-3′

Both primers contain a 5′-phosphorothioate residue (indicated by asterisk). NEB LongAmp DNA polymerase is used to amplify according to manufacturer's instructions. The expected length and specific production of linear DNA should be confirmed visually using gel electrophoresis of a small sample of the PCR reaction. The linear DNA can then be purified using commercial silica-column kits from the remaining PCR reaction(s).

Next, gold nanoparticles are coated with the linear silencing insertion DNA and these will be used for bombardment. In order to prepare tomato explants for bombardment, sterilised tomato seeds are vernalised at 4° C. for 48 hours then germinated on sterile media at 25° C. with a 16:8 hour light:dark cycle. After 8-10 days cotyledons are cut into approximate 0.5 cm squares under sterile conditions and arranged tightly abaxial side-up. These explants can then be used for bombardment essentially as described in: Vishnevetsky, J., White, T. L., Palmateer, A. J., Flaishman, M., Cohen, Y., Elad, Y., Perl, A. (2011). Improved tolerance toward fungal diseases in transgenic Cavendish banana (Musa spp. AAA group) cv. Grand Nain. Transgenic Research, 20(1), 61-72. https://doi.org/10.1007/s11248-010-9392-7.

To select for transformed tomato plantlets, the explants are transferred to media with 40 μg/L chlorsulfuron two weeks after bombardment. After two further weeks explants are transferred to fresh media+40 μg/L chlorsulfuron in tissue culture plates. Thereafter explants are refreshed every 4 weeks onto media+40 μg/L chlorsulfuron in Phytatrays (Sigma-Aldrich, USA). Healthy plantlets containing meristems are sampled. To confirm the insertion, tissue from plantlets is sampled with tweezers and stored in Thermo Scientific Phire dilution buffer. PCR using Thermo Scientific Plant Phire polymerase will take place using this dilution buffer. The presence of the insertion is confirmed using the primers below:

>Sl_GEiGS™_geno_F1
(SEQ ID NO: 29)
5′-GGTAACTCATTTGGGATGGTA-3′
>Sl_GEiGS™_geno_R1
(SEQ ID NO: 30)
5′-GAACGGTCGCTACGATTAC-3′

Plantlets with positive genotyping PCR results (a 103 bp band is produced) are acclimatised to greenhouse conditions, transferred to soil and grown to produce seed. To identify the location of the insertion in the tomato genome Illumina whole-genome sequencing is performed as described above.

### Example 5—Confirmation of Genome-Integrated Silencing Insertion In-Planta

To confirm an in-planta effect of a genome-integrated silencing insertion, this example tests (1) the genome integration of the silencing insertion of Example 2; and (2) a reduction in expression level of the gene targeted by the silencing RNA expressed from the silencing insertion.

To illustrate this, Arabidopsis is transformed with an insertion construct containing the native miR173 gene cassette modified to target the phytoene desaturase gene (PDS3), as in Example 2. PDS3 is an important enzyme for carotenoid biosynthesis, and carotenoids are required for chloroplast membrane stabilisation and chlorophyll accumulation [Qin et al., Cell Res (2007) 17:471]. PDS3 is an established visual reporter for gene silencing: loss of PDS function results in an albino phenotype [Fan et al., Sci Rep (2015) 5:12217]. Therefore, downregulation of the PDS3 transcript by the insertion construct is indicated by the bleaching phenotype. An identical control plasmid is used, but containing wild-type miR173, rather than the modified version of the insertion or containing a modified miR173 version with no specific target in Arabidopsis (e.g targeting GFP). The plasmids are transformed using Agrobacterium, essentially as described in Example 4 (Prior to Agrobacterium transformation the insertions must be inserted in between the left and right borders of a binary vector and this can be cloned using any method known in the art).

Transformed Arabidopsis seeds are germinated on media with 100 nM chlorsulfuron as the selective agent. This will prevent growth in seedlings lacking the mutated ALS gene (and therefore lacking the insertion). Plants with good growth can then be genotyped and transferred to soil for seed production. For genotyping of the seedlings, tissue is sampled with tweezers and stored in Thermo Scientific Phire dilution buffer. PCR using Thermo Scientific Plant Phire polymerase will take place using the dilution buffer containing leaf tissue from germinated seedlings. The presence of the experimental plasmids will be confirmed using the primers below:

>At_GEiGS ™_geno_F2
(SEQ ID NO: 31)
5′-AATCCAGCAGTGGTCAAAA-3′
>At_GEiGS ™_geno_R2
(SEQ ID NO: 32)
5′-CAAGCTCTTTGACAATCCT-3′

The above primer pair will produce a band of 80 bp in plants containing the insertion.

>At_GEiGS ™_dummy_geno_F1
(SEQ ID NO: 33)
5′-GAGCTTCCTCAAAGGGCTGA-3′
>At GEiGS ™_dummy_geno_R1
(SEQ ID NO: 34)
5′-TGGTTACGAATGAATTTAATGGGGA-3′

The above primer pair will produce a band of 312 bp in plants containing the “dummy” (control) plasmid and the tested insertion plasmid. Seedlings with positive genotyping PCR results will be transferred to soil and grown to produce seeds.

To identify the location of the insertion in the Arabidopsis genome, the integration site is identified by sequencing the genomic flanking regions of the construct. This is achieved using thermal asymmetric interlaced (TAIL) PCR, which generates amplification between a known and unknown DNA sequence. TAIL PCR uses arbitrary degenerate (AD) primers with a relatively low Tm (e.g. 45° C.) and nested specific primers with a higher Tm (e.g. 67° C.) within the known sequence, and several PCR runs (Table 1). The first PCR reaction uses the AD primers and the outermost specific primer, and interleaved PCR cycles of high and low Tm to favour specific or non-specific amplification, respectively. The desired products (those between the construct and the flanking region) can be enriched using two subsequent PCR reactions with the nested specific primers (also including the AD primers). Higher efficiency of specific amplification can be achieved by multiple rounds of linear amplification using the specific primer at the start of the PCR, and complementary regions in the AD and specific primers (this biases amplification away from short products, as complementary ends interact to form hairpin structures) (Liu and Chen, 2007). Primers required are shown below:

>AC1
(SEQ ID NO: 35)
5′-ACGATGGACTCCAGAG-3′
>LAD1-1
(SEQ ID NO: 36)
5′-ACGATGGACTCCAGAGCGGCCGC(G/C/A)N(G/C/A)
NNNGGAA-3′
>LAD1-2
(SEQ ID NO: 37)
5′-ACGATGGACTCCAGAGCGGCCGC(G/C/T)N(G/C/T)
NNNGGTT-3′
>LAD1-3
(SEQ ID NO: 38)
5′-ACGATGGACTCCAGAGCGGCCGC(G/C/A)(G/C/A)N
(G/C/A)NNNCCAA-3′
>LAD1-4
(SEQ ID NO: 39)
5′-ACGATGGACTCCAGAGCGGCCGC(G/C/T)(G/A/T)N
(G/C/T)NNNCGGT-3′
>At_GEiGS ™_TAIL_F1
(SEQ ID NO: 40)
5′-TAAGTACTTTGACAATCCAGCCAATCCAGCAGTGGT-3′
>At_GEiGS ™_TAIL_F2
(SEQ ID NO: 41)
5′-GACAATCCAGCCAATCCAGCAGTGGTCAAAAAAG-3′
>At_GEiGS ™_TAIL_F3
(SEQ ID NO: 42)
5′-CTCTGCTGATTGGCAGGATTGTCAAAGAGCTTGCTC-3′

TABLE 1
Primer and template requirements for different PCRs in the hiTAIL-PCR

| HiTAIL-PCR | | |
|---|---|---|
| reaction | Primers | Template |
| Pre-amplification | One or two of [LAD1-1, LAD1-2, | Arabidopsis genomic DNA |
| | LAD1-3, LAD1-4], and | |
| | At_GEiGS ™_TAIL_F1 | |
| Primary TAIL | AC1 and At_GEiGS ™_TAIL_F2 | Product of pre-amplification |
| | | reaction (40x dilution) |
| Secondary TAIL | AC1 and At_GEiGS ™_TAIL_F3 | Product of primary TAIL |
| | | reaction (10x dilution) |

To identify the insertion site, PCR products from the secondary TAIL-PCR will be blunt-end cloned using commercial kits and Sanger sequenced (Liu, Y. G., & Chen, Y. (2007). High efficiency thermal asymmetric interlaced PCR for amplification of unknown flanking sequences. BioTechniques, 43(5), 649-656. https://doi.org/10.2144/000112601).

To assay the phenotypic effect of the silencing insertion, Arabidopsis plants transformed with the insertion construct are compared to plants transformed with the dummy construct. Comparison demonstrates that only plants transformed with the silencing insertion construct display bleaching. Quantitative comparison between the groups is achieved by imaging the plants and using software such as ImageJ to quantify the size of bleached areas between groups. RNA-seq or qRT-PCR are also performed on plants of both groups to compare the expression level of PDS3. PDS3 expression is lower in plants transformed with the silencing insertion construct.

### Example 6—Exemplary Uses of Constructs that Additionally Comprise Sequence Encoding an RNA-Guided DNA Endonuclease and Sequence Encoding a Guide RNA Targeted to the Endogenous Silencing Sequence

In preferred embodiments of the invention, the silencing insertion is introduced into the eukaryotic cell in a construct that additionally comprises sequence encoding an RNA-guided DNA endonuclease and sequence encoding a guide RNA targeted to the endogenous silencing sequence. Transformation using such constructs can provide various potential scenarios, as discussed below.

Scenario 1: Construct Integration, NHEJ Editing of all Endogenous ncRNA Alleles

In this scenario, the transgenic silencing insertion cassette, including sequence encoding an RNA-guided DNA endonuclease and sequence encoding a guide RNA, is expressed from the integrated DNA, preferably integrated T-DNA. The endonuclease, which is preferably CRISPR/CAS9, and sgRNA expression drive the gene editing of all target ncRNA loci that the silencing insertion is based on, resulting in indels. In this scenario, there is no HDR event (no swapping event); hence, non-transgenic plants carrying the silencing insertion at the endogenous silencing sequence locus cannot be generated from such plants, because the loci cannot be re-targeted by the provided sgRNA, however, these plants can be used to study the silencing insertion activity (expressed from the T-DNA). There is no point to continue screening for HDR events in subsequent generations due to the inactivity of the sgRNA in targeting the desired loci. Note: the introduction of the silencing insertion to the edited wt locus (endogenous silencing sequence), can be executed through homologous recombination (HR) in the future generations, however such events are very rare and difficult to detect.

Scenario 2: Construct Integration, No- or Partial NHEJ Editing of Endogenous ncRNA Alleles

In this scenario, similar to scenario 1, the transgenic silencing insertion cassette, including sequence encoding an RNA-guided DNA endonuclease and sequence encoding a guide RNA, is expressed from the integrated DNA, preferably integrated T-DNA. However, the expression of the endonuclease, which is preferably CRISPR/CAS9, and sgRNA did not result in the editing of all (or none of) the target ncRNA loci that the silencing insertion is based on. Hence, some of the loci do not contain indels, and they can still be targeted by the sgRNA provided. In this scenario, there is no HDR event (no swapping event); hence, plants carrying the silencing insertion at the endogenous silencing sequence locus cannot be generated from such tested plants. However, these plants can be used to study the silencing insertion activity (expressed from the T-DNA). In case no- or partial NHEJ editing of endogenous ncRNA alleles occurred, HDR could be screened for in subsequent generations where the editing machinery can still be active. Note: the introduction of the silencing insertion to the wt locus (endogenous silencing sequence), can be executed through homologous recombination, in future generations, however such events are very rare and difficult to detect.

Scenario 3: Construct Integration, HDR-Editing of Endogenous ncRNA Alleles

In this scenario, the transgenic silencing insertion cassette, including sequence encoding an RNA-guided DNA endonuclease and sequence encoding a guide RNA, is expressed from the integrated DNA, preferably integrated T-DNA, as in scenarios 1 and 2. In addition, the expression of the endonuclease, which is preferably CRISPR/CAS9, and sgRNA, together with the presence of the silencing insertion sequence as a DONOR template, result in HDR of all (or some of) the ncRNA loci that the silencing insertion is based on. Hence, the silencing RNA transcript is expressed from the T-DNA (originally inserted silencing insertion) and from the swapped ncRNA allele (non-transgenic). Plants carrying the silencing insertion only at the endogenous silencing sequence locus (which are non-transgenic) cannot be generated from such tested plants, however, these plants can be used to study the silencing insertion activity (expressed from the T-DNA). In subsequent generations the transgenic T-DNA cassette (and the originally inserted silencing insertion) can be crossed out, resulting in non-transgenic plants expressing the silencing insertion from the endogenous silencing sequence locus. Note: the introduction of the silencing insertion to the wt locus (endogenous silencing sequence), can be executed through homologous recombination, however such events are very rare and difficult to detect.

Scenario 4: Construct is not Integrated, NHEJ Editing of all or Some of Endogenous ncRNA Alleles

In this scenario, the silencing insertion, including sequence encoding an RNA-guided DNA endonuclease and sequence encoding a guide RNA, is not integrated, and therefore, the silencing insertion cassette is not expressed. The transient expression of the endonuclease, which is preferably CRISPR/CAS9, and sgRNA drive the gene editing of all or some of the ncRNA loci that the silencing insertion is based on, resulting in indels. There is no point to continue screening for events in subsequent generations since the gene editing machinery and DONOR template are missing. However, this is a positive proof for the activity and efficiency of the gene editing machinery.

Scenario 5: Construct is not Integrated (Transient Expression), No NHEJ is Observed Endogenous ncRNA Alleles

In this scenario, the silencing insertion, including sequence encoding an RNA-guided DNA endonuclease and sequence encoding a guide RNA, is not integrated, and therefore, the silencing insertion cassette is not expressed. In addition, the transient expression of the endonuclease, which is preferably CRISPR/CAS9, and sgRNA, did not result in gene editing of none of the ncRNA loci that the silencing insertion is based on. Hence, the silencing RNA transcript is absent and there is no point in further testing these plants. This might point to the need of improved design of the experiment (e.g., different sgRNA, different nuclease, etc.).

Scenario 6: Construct is not Integrated (Transient Expression), HDR-Editing of Endogenous ncRNA Alleles

In this scenario, the silencing insertion, including sequence encoding an RNA-guided DNA endonuclease and sequence encoding a guide RNA, is not integrated, and therefore, the silencing insertion cassette is not expressed. However, the transient expression of the endonuclease, which is preferably CRISPR/CAS9, and sgRNA, together with the presence of the silencing insertion sequence as a DONOR template, result in HDR of all (or some of) the ncRNA loci that the silencing insertion is based on, as in scenario 3. Hence, the silencing RNA transcript is expressed only from the swapped ncRNA allele. Non-transgenic plants expressing the silencing insertion are generated from such tested plants Note: the introduction of the silencing insertion to the wt locus, can be executed through homologous recombination, however such events are very rare and difficult to detect.

### Scenario 7: Construct is Partially Integrated, Including Only all or Part of the GEiGS-Insertion Cassette

In this scenario, the silencing insertion, including sequence encoding an RNA-guided DNA endonuclease and sequence encoding a guide RNA, is not integrated as a whole, but only a part of, or the complete silencing insertion cassette. Therefore, silencing insertion can be expressed from the location of the random integration. Plants carrying the silencing insertion at a random locus are generated from such tested transgenic plants. Future generations might introduce of the silencing insertion to the wt locus through homologous recombination, which is rare, and will result in non-transgenic plants expressing the silencing insertion from the endogenous silencing insertion.

Scenarios can be combined, through activity in different loci, multiple insertions, or in a heterozygous way. For example:

Scenario 8: Construct Integration, Partial NHEJ Editing of Endogenous ncRNA Alleles and HDR-Editing of Endogenous ncRNA Alleles;

This can occur through combination of scenario 2 (partial NHEJ), which results in indel in of the target gene in one locus, and scenario 3, which results in HDR, in another one. This will result in expression of the silencing RNA from the originally inserted silencing insertion in the integrated DNA, which is preferably a T-DNA fragment, and from the swapped ncRNA allele (endogenous silencing sequence). In subsequent generations the transgenic T-DNA cassette (and the originally inserted silencing insertion) can be crossed out, as described in scenario 3, resulting in non-transgenic plants expressing the silencing insertion from the endogenous silencing sequence locus. Note: the introduction of the silencing insertion to the wt locus, can be executed through homologous recombination, however this is very rare.

Scenario 9: Construct is not Integrated, Partial NHEJ Editing of Endogenous ncRNA Alleles and HDR-Editing of Endogenous ncRNA Alleles

This can occur through combination of scenario 4 (partial NHEJ), which results in indel in some of the target gene in one locus, and scenario 6, which results in HDR, in another one. This will result in expression of the silencing insertion from the swapped ncRNA allele. Hence, the silencing RNA transcript is expressed only from the swapped ncRNA allele. Non-transgenic plants are generated from such tested plants Note: the introduction of the silencing insertion to the wt (endogenous silencing sequence) locus, can be executed through homologous recombination, however such events are very rare and difficult to detect.

TABLE 2
summary of scenarios

| | | | | | GEiGS ™- | | |
|---|---|---|---|---|---|---|---|
| | | | NHEJ | HDR | insertion | | |
| | | | editing of | editing of | expression/ | HDR screen | Application in |
| | | T-DNA | WT ncRNA | WT ncRNA | GEiGS ™-HDR | in subsequent | transformed |
| # | Scenario | integration | scaffold | scaffold | expression | generations | generation |
| 1 | T-DNA integration, | Yes | Yes | No | GEiGS ™- | No. Unlikely to | Transgenic GEiGS ™- |
| | NHEJ editing of | | (in all | | insertion only | find HDR-editing | mimic (GEiGS ™ is |
| | all endogenous | | alleles) | | | in subsequent | expressed from the |
| | ncRNA alleles | | | | | generations. HR | natural promoter and |
| | | | | | | editing might | terminator of |
| | | | | | | potentially occur, | the ncRNA scaffold) |
| | | | | | | but such events | |
| | | | | | | are very rare and | |
| | | | | | | difficult to detect | |
| 2a | T-DNA integration, | Yes | Yes | No | GEiGS ™- | Yes | Transgenic GEiGS ™- |
| | NHEJ editing of | | (in some | | insertion | | mimic + screen in |
| | some (not all) | | alleles) | | only | | subsequent |
| | endogenous ncRNA | | | | | | generations for HDR |
| | alleles | | | | | | |
| 2b | T-DNA integration, | Yes | No | No | GEiGS ™- | Yes | Transgenic GEiGS ™- |
| | no-NHEJ editing | | | | insertion | | mimic; Screen in |
| | of endogenous | | | | only | | subsequent |
| | ncRNA alleles | | | | | | generations for HDR |
| 3 | T-DNA integration, | Yes | No | Yes | GEiGS ™- | Yes, as well, to | Analysis of combined |
| | HDR-editing of | | | | insertion and | generate homozygous | transgenic GEiGS ™- |
| | endogenous ncRNA | | | | GEiGS ™-HDR | or heterozygous | mimic and GEiGS ™-HDR; |
| | alleles | | | | | lines, as required. | Screen in subsequent |
| | | | | | | In addition, T-DNA | generations for non- |
| | | | | | | transgene can be | transgenic GEiGS ™-HDR |
| | | | | | | breed out. | |
| 4 | T-DNA is not | No | Yes | No | None | No. Required | Evidence for |
| | integrated (transient | | (all or | | | components to | efficient and active |
| | expression), NHEJ | | some) | | | introduce GEiGS ™ | design of the GE |
| | editing of all or | | | | | are absent. | machinery and method |
| | some of endogenous | | | | | | |
| | ncRNA alleles | | | | | | |
| 5 | T-DNA is not | No | No | No | None | No. Required | Might point to |
| | integrated (transient | | | | | components to | problematic design |
| | expression), no NHEJ | | | | | introduce GEiGS ™ | of the system or |
| | is observed | | | | | is absent. | its set up |
| | endogenous ncRNA | | | | | | |
| | alleles | | | | | | |
| 6 | T-DNA is not | No | No | Yes | GEiGS ™-HDR | GEIGS ™-HDR | GEiGS ™-HDR plant |
| | integrated (transient | | | | | is achieved in | analysis; Plant line |
| | expression), HDR- | | | | | transformed | establishment |
| | editing of endogenous | | | | | generation, but | (heterozygous and |
| | ncRNA alleles | | | | | should be maintain | homozygous); |
| | | | | | | through screen in | |
| | | | | | | future generations, | |
| | | | | | | to generate | |
| | | | | | | homozygous or | |
| | | | | | | heterozygous lines, | |
| | | | | | | as required. | |
| 7 | T-DNA is partially | Yes | No | No | GEiGS ™- | No. Required | GEiGS ™-mimic plant |
| | integrated, including | Partial | | | insertion | components to | analysis; Plant line |
| | only all or part of | | | | | introduce GEiGS ™ | establishment |
| | the GEiGS-insertion | | | | | are absent. | (heterozygous and |
| | cassette | | | | | | homozygous); |
| 8 | T-DNA integration, | Yes | Yes (in | Yes | GEiGS ™- | Yes; as well, to | Analysis of combined |
| (2b + 3) | partial NHEJ editing | | some) | | insertion and | generate homozygous | transgenic GEiGS ™- |
| | of endogenous ncRNA | | | | GEiGS ™-HDR | or heterozygous | mimic and GEiGS ™-HDR; |
| | alleles and HDR- | | | | | lines, as required. | Screen in subsequent |
| | editing of endogenous | | | | | In addition, T-DNA | generations for |
| | ncRNA alleles | | | | | transgene can be | non-transgenic GEiGS ™- |
| | | | | | | breed out. | HDR (heterozygous and |
| | | | | | | | homozygous for HDR and |
| | | | | | | | edited, non-edited and |
| | | | | | | | HDR locus); |
| 9 | T-DNA is not | No | Yes (in | Yes (in | GEiGS ™-HDR | GEIGS ™-HDR is | GEiGS ™-HDR plant |
| (4 + 6) | integrated, partial | | some) | some) | | achieved in current | analysis; Plant line |
| | NHEJ editing of | | | | | generation, but | establishment |
| | endogenous ncRNA | | | | | should be maintain | (heterozygous and |
| | alleles and HDR- | | | | | through screen in | homozygous for HDR |
| | editing of endogenous | | | | | future generations, | and edited, non-edited |
| | ncRNA alleles | | | | | to generate | and HDR locus); |
| | | | | | | homozygous or | |
| | | | | | | heterozygous lines, | |
| | | | | | | as required. | |

### Example 7. Generating Arabidopsis thaliana Plants Carrying a GEiGS™-Insertion Cassette

RNA silencing can be amplified by the production of secondary small interfering RNAs (siRNAs) resulting from processing of a long dsRNA precursor. 22 nt long miRNAs have been shown to trigger secondary siRNA production in an RNA-dependent RNA polymerase (RDR) and Dicer-like (DCL)-dependent manner (Chen et al., 2010; McHale et al., 2013). Mutations in RNA silencing-associated genes, such as DCL and AGO2, have been associated with host susceptibility to viruses, suggesting that RNA silencing is an effective defence mechanism against viral infections (Jaubert et al., 2011; Yang et al., 2004). The inventors hypothesised that an insertion of a 22 nt miRNA scaffold modified to express a GEiGS™-miRNA targeting the Turnip Mosaic Virus (TuMV) genome would therefore induce resistance against TuMV infection in A. thaliana.

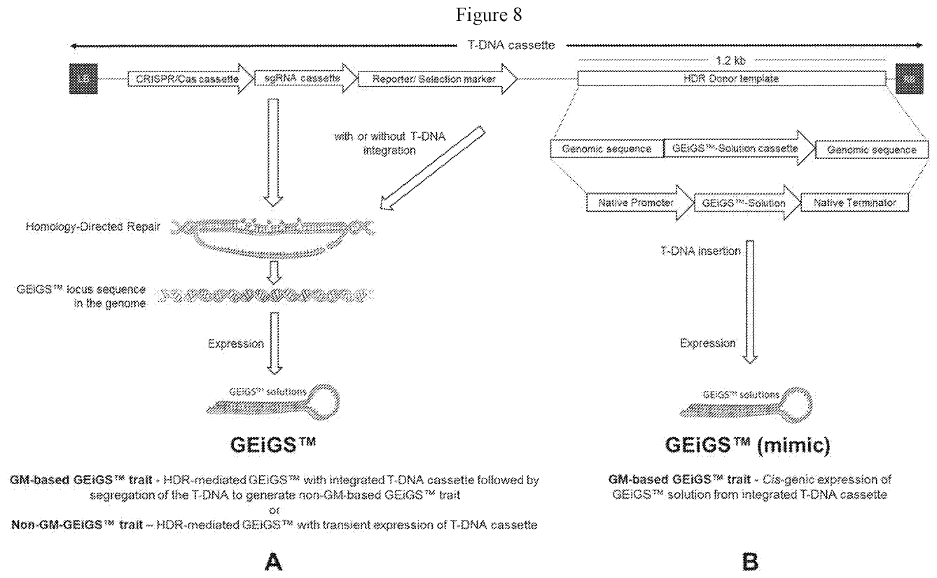

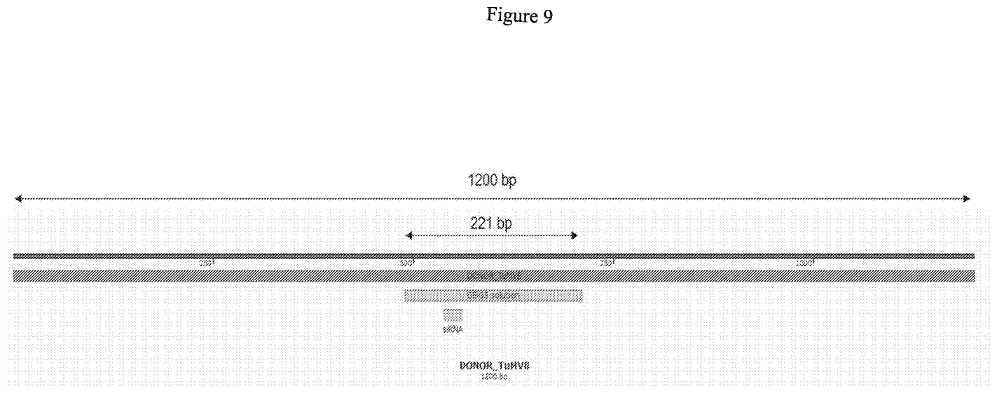

This example describes the process to generate A. thaliana lines carrying a T-DNA that contains (i) a selection cassette; (ii) a gene editing cassette; and (iii) a 22 nt miRNA GEiGS™-Insertion cassette. The insertion cassette can be used as a donor template for CRISPR/Cas-mediated gene editing via HDR or as an expression cassette. The GEiGS™-Insertion cassettes used in this example are 1.2 kb in length, spanning the modified region required to redirect the silencing specificity of the miRNA plus approximately 500 bp upstream and downstream of this site (FIG. 9).

To control for pleiotropic effects of expressing TuMV-specific 22 nt miRNA GEiGS™-Insertion cassettes, a “dummy” control was designed for each modified scaffold. In “dummy” constructs the GEiGS™-miRNA guide and passenger sequences are replaced with a randomised nucleotide sequence which maintains identical RNA secondary structure but is not complementary to TuMV or the A. thaliana transcriptome.

Promoters and terminators of the native 22 nt scaffolds were predicted using bioinformatic analyses as described in Example 1 and confirmed that the GEiGS™-Insertion cassettes contain the required sequences to drive transcription of GEiGS™-miRNAs. Table 3 details the mature miRNAs expected to be expressed by the GEiGS™-Insertion cassettes used in this example (complete sequences can be found in the appendix):

TABLE 3
GEiGS ™-Insertion cassettes used in Examples
7-9.

| | | Tar- | miRNA se- | |
|---|---|---|---|---|
| | miRNA | geting | quence | |
| GEiGS ™_ | scaf- | speci- | (5′-3′, | SEQ |
| Insertion | fold | ficity | guide/ | ID |
| | | | passenger) | NO. |
| TuMV8 | ath- | TuMV | uauuauuucaaagua | SEQ |
| | miR850 | | guugcuggaggcuag | ID |
| | | | uuuacauaguuga | NO: |
| | | | (SEQ ID NO: 43) | 83 |
| Dummy17 | ath- | N/A | uuucgugggguucac | SEQ |
| | miR850 | | uaaucugguuuaguc | ID |
| | | | ggcggaugagugu | NO: |
| | | | (SEQ ID NO: 44) | 84 |
| TuMV10 | ath- | TuMV | uuuauuguucaugua | SEQ |
| | miR162a | | guaggcuucuacaau | ID |
| | | | auuagcgcugaag | NO: |
| | | | (SEQ ID NO: 45) | 85 |
| Dummy19 | ath- | N/A | uuucgugggguucac | SEQ |
| | miR162a | | uaaucucgguuaaug | ID |
| | | | gggcccaaggaag | NO: |
| | | | (SEQ ID NO: 46) | 86 |
| TuMV12 | ath- | TuMV | uuacauguugaugaa | SEQ |
| | miR173 | | cuuccugagggguuc | ID |
| | | | agcgguauguaaa | NO: |
| | | | (SEQ ID NO: 47) | 87 |
| TuMV14 | ath- | TuMV | uuuauuguucaugua | SEQ |
| | miR173 | | guaggcugccacuac | ID |
| | | | agggauagugaau | NO: |
| | | | (SEQ ID NO: 48) | 88 |
| Dummy21 | ath- | N/A | uuucgugggguucac | SEQ |
| | miR173 | | uaaucucagauagug | ID |
| | | | guuuccacggaaa | NO: |
| | | | (SEQ ID NO: 49) | 89 |

### Cloning of Constructs

Binary plasmids for gene editing were assembled using Golden Gate cloning and GEiGS™-Insertion cassettes were then introduced via restriction digestion by PacI/AscI and ligation. Final plasmids were validated by Sanger sequencing.

### Agrobacterium Preparation

Plasmids were transformed into Agrobacterium tumefaciens GV3101 (pMP90, pSoup) through electroporation and kanamycin-resistant colonies were validated by colony PCR, Sanger sequencing and differential digestion before storage as glycerol stocks. Glycerol stocks were streaked on solid LB medium supplemented with Kanamycin 50 mg/L, Rifampicin 50 mg/L, Gentamycin 50 mg/L and Tetracyclin 5 mg/L and the plates were incubated for two days at 28° C. Ten colonies were inoculated in 10 mL of LB supplemented with Kanamycin 50 mg/L, Rifampicin 50 mg/L, Gentamycin 50 mg/L and Tetracyclin 5 mg/L and the cultures were incubated overnight at 28° C., 200 rpm. On the next day, 10 mL of culture was used to inoculate 1 L of LB medium supplemented with Kanamycin 50 mg/L, Rifampicin 50 mg/L, Gentamycin 50 mg/L and Tetracyclin 5 mg/L. Cultures were incubated overnight at 28° C., 200 rpm. On the next day, the OD_{600 nm} of the bacteria was measured using a NanoDrop2000 and normalised to 1.0 in 5% sucrose supplemented with 20 μM acetosyringone. Cultures were incubated at room temperature for 3 h in the dark with gentle agitation.

A. thaliana Transformation Via Floral Dipping

A. thaliana ecotype Columbia-0 (Col-0) plants were sown and grown in short day conditions for 6 weeks (8 hours light and 16 hours dark). Plants were transferred to long day conditions (16 hours light and 8 hours dark) for 10 days to induce flowering. Immediately before dipping, A. tumefaciens cell suspensions were supplemented with 0.05% Silwet L-77 and mixed well. Plant inflorescences were submerged in the cell suspension and subjected to vacuum for 1 min. Vacuum was quickly released and the plants were stored horizontally inside a bag to retain moisture for 24 hours recovery. Plants were kept in long day conditions (16 hours light and 8 hours dark) for a further 6 weeks. Once the plants flowered and the first siliques started to dry, plants were bagged and T1 seeds were harvested.

### Selection of T1 Plants

Selection plates were prepared by mixing on a Petri dish 20 mL of non-sterile silicon dioxide (Sigma 84880) and 10 mL of ¼ Murashige and Skoog (MS) media (1.1 g/L MS with vitamins—Duchefa M0222, pH 5.8) supplemented or not with hygromycin B 40 mg/L. Excess liquid was soaked up with a piece of tissue paper. A. thaliana seeds were surface-sterilised by exposure to chlorine gas for 2 h. Seeds were sparsely sprinkled onto the humid media, plates were sealed with micropore tape and incubated at 4° C. in the dark for 2-3 days. Following stratification, plates were kept at 22° C. under long day conditions. Three days later, plates were watered with ¼ MS media supplemented or not with hygromycin B 40 mg/L until the surface looked moist. From this point, every 3-4 days plates were watered with ¼ MS media without hygromycin B. Col-0 seeds were used as a control to verify germination and selection efficiency. Fifteen days following stratification, resistant plants were readily visible (Davis et al., 2009).

### Genotyping of T1 Plants

Following two weeks under selection, a small leaf per plant was sampled and stored in 20 μL of dilution buffer (Phire Plant Direct PCR Master Mix, Thermo Scientific). A PCR reaction was performed according to the manufacturer's instructions to amplify the T-DNA region containing the GEiGS™-Insertion cassette using the following primers and conditions (Tables 4-5):

>G1177:
(SEQ ID NO: 50)
5′-TCACATGTGCATCCTCTCTGA-3′
>1178:
(SEQ ID NO: 51)
5′-GGCTAGTCCGTTATCAACTTGA-3′

TABLE 4
PCR reaction components

| Component | Volume (μL per reaction) |
|---|---|
| 2x Phire Plant Direct PCR Master Mix | 10 |
| Phire dilution buffer with tissue | 1 |
| 10 μM Forward primer | 1 |
| 10 μM Reverse primer | 1 |
| Water | 7 |

TABLE 5
PCR reaction conditions

| Cycle Step | Temperature (° C.) | Time | | Cycles |
|---|---|---|---|---|
| Initial denaturation | 98 | 5 | min | 1 |
| Denaturation | 98 | 5 | sec | |
| Annealing | 63 | 5 | sec | 35 |
| Extension | 72 | 40 | sec | |
| Final Extension | 72 | 1 | min | 1 |

PCR products were cleaned up using the ExoSAP-IT PCR Product Cleanup Reagent (Thermo Scientific) according to the manufacturer's instructions and sent for Sanger sequencing to confirm the identity of the T-DNA insertion within selected plants.

### T2 Seed Harvest

Successfully genotyped plants were transferred to soil and kept in long day conditions (16 hours light and 8 hours dark) to induce flowering. Once the first siliques started to dry, plants were bagged and T2 seeds were harvested.

### Example 8. Plants Carrying GEiGS™-Insertion Cassettes Show Expression of GEiGS™-miRNAs

This example demonstrates that GEiGS™-Insertion cassettes are capable of driving expression of GEiGS™-miRNAs.

A. thaliana Culture

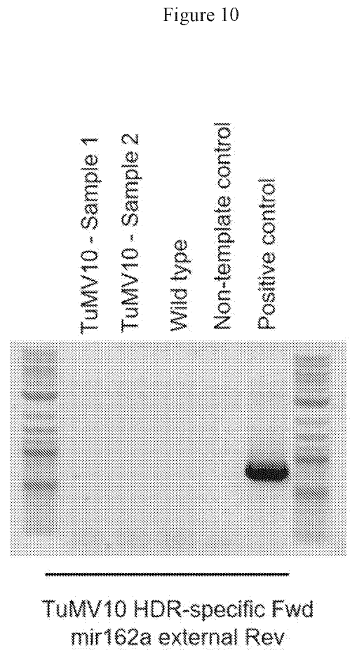

T2 A. thaliana plants were germinated, selected in hygromycin and genotyped as described in Example 7. In addition, the plants were genotyped to ensure the absence of HDR-mediated edits on their native scaffold that could lead to expression of GEiGS™ miRNAs (FIG. 10). PCR reactions were performed using a forward primer specific to the intended HDR edits and a reverse primer external to the HDR donor/GEiGS™-Insertion cassette, i.e. specific to the native scaffold. Reactions were performed according to the manufacturer's instructions using the following primers and conditions (Tables 6-7):

>TuMV10 HDR-specific Fwd:
(SEQ ID NO: 52)
GAAGCCTACTACATGAACA
>mir162a external Rev:
(SEQ ID NO: 53)
TAGTCCACTGGTTAATCAGAGA

TABLE 6
PCR reaction components

| Component | Volume (μL per reaction) |
|---|---|
| 2x Phire Plant Direct PCR Master Mix | 10 |
| Phire dilution buffer with tissue | 1 |
| 10 μM Forward primer | 1 |
| 10 μM Reverse primer | 1 |
| Water | 7 |

TABLE 7
PCR reaction conditions

| Cycle Step | Temperature (° C.) | Time | | Cycles |
|---|---|---|---|---|
| Initial denaturation | 98 | 5 | min | 1 |
| Denaturation | 98 | 5 | sec | |
| Annealing | 65 | 5 | sec | 35 |
| Extension | 72 | 20 | sec | |
| Final Extension | 72 | 1 | min | 1 |

Following genotype confirmation, plants were transferred to soil and kept in long day conditions (16 hours light and 8 hours dark) for 28 days. Four leaves of each plant were harvested and flash-frozen in liquid nitrogen. Samples were kept at −80° C. until RNA extraction.

### RNA Extraction

Samples were ground twice with 5 mm stainless steel beads in a tissue lyser (TissueLyserII, Qiagen, 24 Hz, 30 see) and resuspended in lysis buffer (100 mM Tris-HCl, pH 9.5; 150 mM NaCl, 1.0% Sarkosyl, 1% β-mercaptoethanol). Samples were incubated with agitation for 5 minutes at room temperature, spun down at 15,000 g for 5 min, and the collected supernatant was mixed with chloroform (1:1). Tubes were vortexed for 2 min and water-saturated phenol was added (1:1:1) followed by vortexing for 2 min. Samples were spun down at 15,000 g for 15 min and the aqueous phase was recovered and mixed with an equal volume of chloroform for a second cleaning step as described above. The aqueous phase was recovered and mixed well with 1100 μL of isopropanol and 90 μL of 3M sodium acetate (pH 5.2). Samples were mixed by gentle inversion and stored overnight at −20° C. for RNA precipitation. On the next day, samples were spun down at 15,000 g for 20 min and the supernatant was discarded. The RNA pellet was washed twice with 1 mL of 75% ethanol followed by centrifugation at 15,000 g for 5 min. The ethanol was completely removed, and the pellets were left to air dry for 7-10 min. Samples were resuspended in 50 μL of nuclease-free water. Once the RNA pellet was completely dissolved, 1 mL of TRIzol (Thermo Scientific) was added, and the samples were incubated with agitation for 15 minutes at room temperature; following the incubation, 200 μL of chloroform were added to each sample. The samples were vortexed for 2 min and spun down at 15,000 g for 15 minutes. The supernatant was collected and mixed with an equal volume of absolute ethanol (1:1), and 650 μL was loaded in a column of the Direct-zol RNA Miniprep kit (Zymo Research). Samples were processed according to the manufacturer's instructions and eluted in 35 μL of DEPC-treated water. RNA integrity was determined using a Bioanalyzer (Agilent) and samples were stored at-80° C.

SRNA Sequencing (sRNA-Seq)

Small RNA libraries (Novogene) were produced using the NEB Next Multiplex Small RNA Library Prep Set for Illumina (NEB) following the manufacturer's protocol. Indices were included to multiplex samples. As small RNAs have phosphoric acid group at 5′ end and hydroxyl group at 3′ end, adapters were directly ligated to small RNA fragments. Libraries were constructed via adapter ligation, reverse transcription, and PCR enrichment. After size selection of insertions between 18-40 bp, single-end sequencing was performed to produce 50 bp reads (SE50) using the Illumina NovaSeq 6000 SP flowcell.

sRNA-Seq Analyses

Raw sequencing data was processed into clean data following these steps: (1) Eliminating reads of which more than 50% bases have a base quality score lower than 5; (2) Eliminating reads containing N>10%; (3) Eliminating reads with 5′ primer contaminants; (4) Eliminating reads without 3′ primer and reads without the insert tag; (5) Trimming 3′ primer sequence; (6) Eliminating reads with polyA/T/G/C. Small RNA adapter sequences are as follows: RNA 5′ Adapter (RA5): 5′-GTTCAGAGTTCTACAGTCCGACGATC-3′ (SEQ ID NO: 54) and RNA 3′ Adapter (RA3): 5′-AGATCGGAAGAGCACACGTCT-3′ (SEQ ID NO: 55).

Clean sequencing data was first mapped to the TAIR 10 reference genome sequence using the STAR alignment tool with parameters set to end-to-end 100% sequence match and up to 1000 multimaps allowed (Dobin et al., 2013). All the clean reads were also mapped to GEiGS™-Insertion transcript sequences using the same method. Lastly, all unmapped reads of the first run were mapped to GEiGS™-Insertion transcript sequences using the same method.

In plants, viral infection leads to accumulation of virus-derived siRNAs (vsiRNAs) which are preferentially 21-22 nucleotides in length (Xia et al., 2014). In order to unambiguously detect expression of TuMV-specific miRNAs derived from transcriptional activity of GEiGS™-Insertion cassettes, sRNA-seq analyses were performed in non-infected GEiGS™-Insertion plants. As a control for the detection of TuMV-derived siRNAs, infected wild type plants were also sequenced. As shown in Table 8, expression of a TuMV-specific sRNA was detected in RNA extracted from infected wild type plants, as well as non-infected GEiGS™-Insertion TuMV10 plants. As expected, no expression of TuMV-specific sRNAs was detected in non-infected GEiGS™-Insertion Dummy plants. This suggests that GEiGS™-Insertion cassettes are transcriptionally active and could lead to resistance against TuMV infection. Transcriptional activity of additional GEiGS™-Insertion cassettes (TuMV12) was similarly confirmed (data not shown).

TABLE 8
Expression of TuMV-specific sRNAs in GEiGS ™-Insertion
and wild type plants.

| | | | Expression of | |
|---|---|---|---|---|
| | GEiGS ™- | TuMV- | TuMV-specific | Expression of |
| Sample | Insertion | infected | sRNA | dummy sRNA |
| 1 | TuMV10 | − | + | − |
| 2 | TuMV10 | − | + | − |
| 3 | Dummy | − | − | + |
| Col-0 | N/A | + | + | − |

### Example 9. A. thaliana Plants Carrying GEiGS™-Insertion Cassettes Show Resistance Against TuMV Infection

Following confirmation of transcriptional activity from TuMV10 and Dummy GEiGS™-Insertion, plants carrying these cassettes in their genome were challenged with TuMV. This example demonstrates that the presence of a transcriptionally active TuMV-specific GEiGS™-Insertion cassette in A. thaliana leads to resistance against TuMV.

The infectious clone of TuMV used in this example is a binary plasmid containing the full-length cDNA form of the viral genome in its T-DNA region, and A. tumefaciens was used as a delivery method. Under a suitable promoter, the DNA form of a viral genome can be transcribed into a viral RNA, thus acting as a trigger for infection establishment (Boyer & Haenni, 1994). The TuMV isolate used was tagged with a green fluorescent protein (GFP) reporter, allowing rapid visualisation of viral spread when A. thaliana plants were illuminated with UV light.

### Cloning of TuMV-GFP Construct

The CDS of TuMV, isolate UK1, carrying a GFP gene inserted between the NIb and the capsid protein (Touriño et al., 2008) was linearised through SmaI/ApaI digestion. pICSL4723 vector was amplified using the following primers and conditions (Tables 9-10):

>1401:
(SEQ ID NO: 56)
5′-ATGTTTGAACGATCGGGCCCaagggacacgaagtgatccg-3′
>1402:
(SEQ ID NO: 57)
5′-CTCCACCATGTTCCCGGGggcacagagtgttcaacccc-3′

TABLE 9
PCR reaction components

| Component | Volume (μL per reaction) |
|---|---|
| CloneAmp HiFi PCR Premix | 12.5 |
| Template | 100 ng |
| 10 μM Forward primer | 1 |
| 10 μM Reverse primer | 1 |
| Water | Up to 25 μL |

TABLE 10
PCR reaction conditions

| Cycle Step | Temperature (° C.) | Time | | Cycles |
|---|---|---|---|---|
| Initial denaturation | 98 | 30 | sec | 1 |
| Denaturation | 98 | 10 | sec | |
| Annealing | 55 | 15 | sec | 34 |
| Extension | 72 | 1 | min | |
| Final Extension | 72 | 1 | min | 1 |

The TuMV CDS was cloned into the T-DNA region of the pICSL4723 binary vector using In-Fusion cloning (Takara Bio Europe), according to the manufacturer's instructions. Final plasmids were verified by Sanger sequencing.

### Agrobacterium Preparation

Plasmids were transformed into A. tumefaciens AGL-1 through electroporation and kanamycin-resistant colonies were validated by colony PCR, Sanger sequencing and differential digestion before storage as glycerol stocks. Glycerol stocks were streaked on solid LB medium supplemented with Rifampicin 50 mg/L, Carbenicillin 50 mg/L and Kanamycin 50 mg/L and the plates were incubated for two days at 28° C. Ten colonies were inoculated in 5 mL of LB supplemented with Rifampicin 50 mg/L, Carbenicillin 50 mg/L and Kanamycin 50 mg/L and the cultures were incubated overnight at 28° C., 200 rpm. On the next day, 150 μL of culture was used to inoculate 10 mL of LB medium supplemented with Rifampicin 50 mg/L, Carbenicillin 50 mg/L and Kanamycin 50 mg/L. Cultures were incubated overnight at 28° C., 200 rpm. On the next day, the OD_{600 nm} of the bacteria was measured using a NanoDrop2000 and normalised to 0.5 in infiltration medium (10 mM MES, 10 mM MgCl₂, 200 μM acetosyringone). Cultures were diluted 1:100 in infiltration medium and incubated at 28° C., 200 rpm for 3 hours in the dark.

### TuMV Bioassay

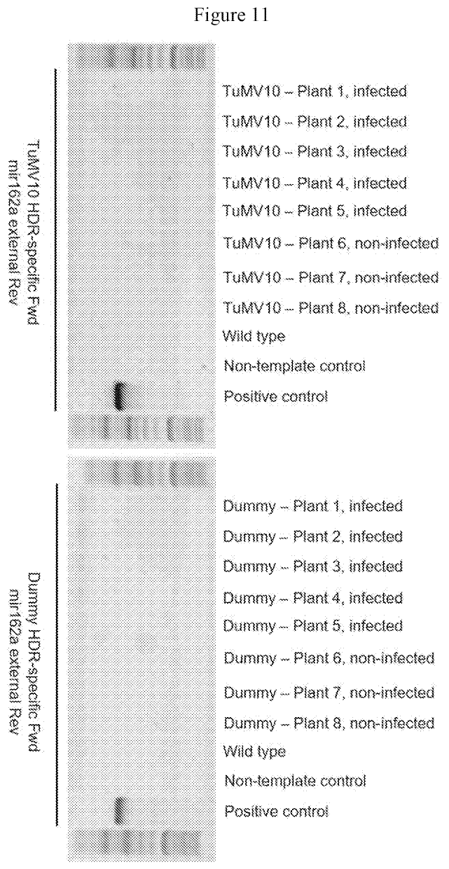

T2 A. thaliana plants were germinated, selected in hygromycin and genotyped as described in Example 7. In addition, the plants were genotyped to ensure the absence of HDR-mediated edits on their native scaffolds that could lead to expression of GEiGS™ miRNAs (FIG. 11). PCR reactions were performed using a forward primer specific to the intended HDR edits and a reverse primer external to the HDR donor/GEiGS™-Insertion cassette, i.e. specific to the native scaffold. Reactions were performed according to the manufacturer's instructions using the following primers and conditions (Tables 11-12):

>TuMV10 HDR-specific Fwd:
(SEQ ID NO: 52)
GAAGCCTACTACATGAACA
>Dummy HDR-specific Fwd:
(SEQ ID NO: 58)
GATTAGTGAACCCCACGAA
>mir162a external Rev:
(SEQ ID NO: 53)
TAGTCCACTGGTTAATCAGAGA

TABLE 11
PCR reaction components

| Component | Volume (μL per reaction) |
|---|---|
| 2x Phire Plant Direct PCR Master Mix | 10 |
| Phire dilution buffer with tissue | 1 |
| 10 μM Forward primer | 1 |
| 10 μM Reverse primer | 1 |
| Water | 7 |

TABLE 12
PCR reaction conditions

| Cycle Step | Temperature (° C.) | Time | | Cycles |
|---|---|---|---|---|
| Initial denaturation | 98 | 5 | min | 1 |
| Denaturation | 98 | 5 | sec | |
| Annealing | 65 | 5 | sec | 35 |
| Extension | 72 | 20 | sec | |
| Final Extension | 72 | 1 | min | 1 |

Following genotype confirmation, plants were transferred to soil and kept in long day conditions (16 hours light and 8 hours dark) for 4 weeks in total (pre-bolting stage). Three leaves were gently pricked on the abaxial side with a P10 pipette tip and a 2 μL droplet of A. tumefaciens suspension was placed on top of the wound. The leaves were gently pricked again where the droplet was placed. Mock inoculations were performed with an AGL-1 A. tumefaciens suspension without a binary vector. Inoculated plants were kept in a Containment Level 2 growth facility in long day conditions (16 hours light and 8 hours dark). Plants were photographed with a digital camera (Lumix, Leica Lens-DC Vario-ELMAR 1:3.3-6.4/4.3-129 ASPH, Panasonic) mounted on a tripod with fixed distance and settings (ISO 400, f/8 aperture, 3.2 sec exposure) under UV illumination 7, 11, 15, and 17 days post inoculation (dpi) to investigate GFP spreading.

### GFP Area Analysis

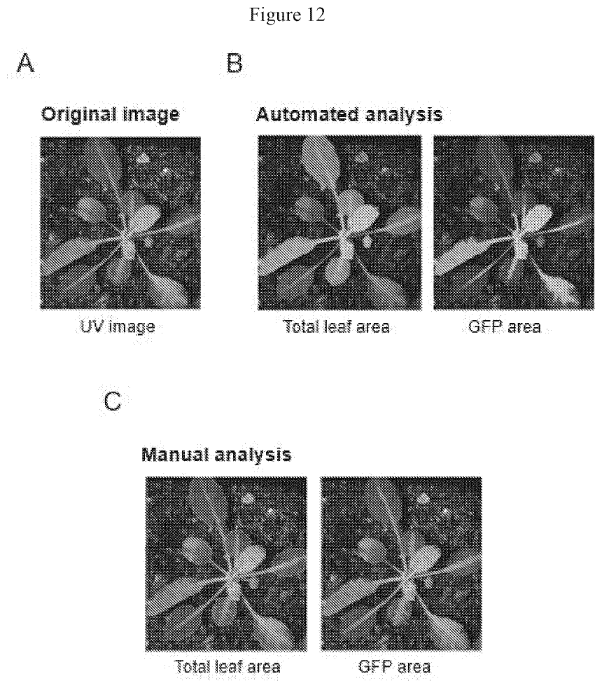

Photographs were analysed to determine the proportion of total leaf area occupied by GFP fluorescence—and thereby the proportion of above ground plant tissue colonised by TuMV. A custom image analysis pipeline was developed in CellProfiler 3.1.9 (Mcquin et al., 2018). Briefly, total leaf area was identified by thresholding (minimum cross-entropy) blue-component grayscale images, identifying objects, filtering small objects, then manually curating the remaining objects. At this point, the objects identified represented a close approximation of the total leaf area, and the pipeline measured this area. An output image overlaying the total leaf area was saved for quality-control. To identify the total area colonised by TuMV:GFP (TuMV area), the total leaf area objects were used to mask the input image, meaning only areas identified as leaves were analysed. Next, the red component grayscale image was subtracted from the green component grayscale image (removing artefacts), the resulting image was thresholded (Otsu, two classes), objects identified, manually curated, then measured and the TuMV:GFP area recorded. An output image overlaying the TuMV:GFP area was saved for quality-control. Images saved for quality control were reviewed manually and those with large errors had the total leaf area and TuMV:GFP area measured manually using ImageJ (FIG. 12). The proportion of TuMV:GFP area over total leaf area was compared between individuals transformed with different GEiGS™-Insertion constructs and their corresponding “dummy” controls and wild-type A. thaliana.

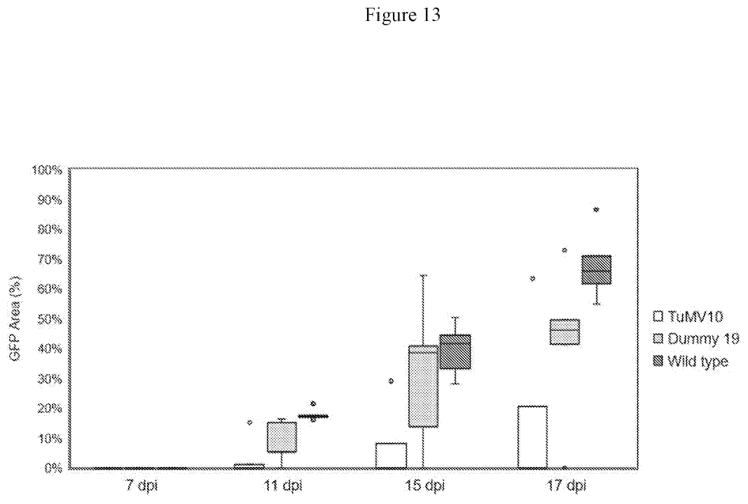

Quantification of the TuMV:GFP area in leaves of infected plants demonstrates that the presence of a TuMV-specific GEiGS™-Insertion cassette leads to reduction in viral spread when compared to the dummy and wild type (Col-0) controls (FIG. 13).

### Sequences

All nucleotide sequences are listed in the 5′-3′ orientation. All protein sequences are listed N-terminus to C-terminus.

### Example 2 Sequences

miR173 WT
Arabidopsis thaliana chromosome 3 sequence (SEQ ID NO: 59)
GenBank: CP002686.1 (5860291-5862937)
miR173 promoter region (SEQ ID NO: 60)
miR173 (WT) ncRNA (SEQ ID NO: 1)
miR173 terminator region (SEQ ID NO: 61)
miR173 (GEiGS ™-Insertion) (SEQ ID NO: 62)
5′-
cagaagccaaatctttggtaaacaaagtaatttttttgccatttgattggttagtataggagaatttaaaaacgacga
taaggtttaggtaaattatttcatttgaaaataattgagcaccgttaataattttcatccataaaataatatttcaaa
gatgatatttgatccccattaaattcattcgtaaccaaaaaaaagttatgaaaaaagagtggtcgtgtgagttgccca
agcaccattataataaaaaataaaataattagcaagtaataaggaataaaatcctgtaattatagctgaaaaaggaaa
aatatttggagaccgtcagattcgaatctgaacaaagcataaaaaagtcaacaaaacttaaagcggcggtctcatcgt
aatctcagcccaataccctattttcctctcccctatataaatactttcttcttctactgatcttcttctcacaaataa
acccaaatatatcaatctactgtgttggtgattaagtactttgacaatccagccaatccagcagtggtcaaaaaagtt
gtagttttcttaaagtctctttcctctgctgattggcaggattgtcaaagagcttgctccctaaacttatctctctga
tgatttaatgttagagatcttcgtaaatctatgtgtttgatagatctgatgcgttttttgagttgatgatttgattat
ttttcactggaaagtatctcattagggtaacgataatgttttatggatttggttgtataacagatccatgaaatcttg
actggttataaaatctgaataatgtatttcaatttggagattcggtgataaaaattactgatttacgaatgacattta
tatcgatagatgagtttgctgatttggttgttaaattgataaatcaaggacatgagaaactgtttttgtatgctaatt
tgtccatggaataaaattgggaggtgaggaccgtgagggtagtcaggaaaccttaatattgaagtt-3′
1002nt
Number of nucleotides changed from the WT sequence: 31 (3.09%)
miR173 (GEiGS ™) promoter region (SEQ ID NO: 60)
Number of nucleotides changed from the WT sequence: 0 (0%)
miR173 (GEiGS ™) ncRNA (SEQ ID NO: 2):
5′-
taagtactttgacaatccagccaatccagcagtggtcaaaaaagttgtagttttcttaaagtctctttcctctgctga
ttggcaggattgtcaaagagcttg-3′
102nt
Number of nucleotides changed from the WT sequence: 31 (30.39%)
miR173 (GEiGS ™) terminator region (SEQ ID NO: 61)
Number of nucleotides changed from the WT sequence: 0 (0%)
Tas3a (WT)
Arabidopsis thaliana chromosome 3 sequence (SEQ ID NO: 63)
GenBank: CP002686.1 (8235653-8236654)
Tas3a promoter region (SEQ ID NO: 64):
Tas3a (WT) ncRNA (SEQ ID NO: 6)
Tas3a terminator region (SEQ ID NO: 65)
Tas3a (GEiGS ™-Insertion) (SEQ ID NO: 66)
5′-
atgaaattaagagccttgtcctagttttgtagtagtattttgttataaaaaaatattgcaattgaaaaataaaggttt
tactgaataccaccaatgtgtgtgttaaactaataaacatatttgtacattacgcgaaacattaattaatttaggaac
gaaggtcaagggattcttttttggcattatcaaatgatataacaatgttaatagttactataagttgtgatattacta
tttttttacattattttgaccattttatgtcttagaagactgtgtgattcccccgcacgcccacaaagtttctattcc
ccacaaatctcagacttttgattactggattttagaaataagaataataacttgctcgaaagagattcgaccttcttt
atcttacttcattttaaagaaaactgataagccattgaataagaacataattcctctttttttttgttagaatgagaa
gagagcaaagaaacttcttaaaatgttataaaactaattaatactaggaagactataagcttgaaatatataaactat
tacagagtaagtactacatatataaaaaacacagtgtgtgtaaacatgatttattttgaaatttgaactttgatgtgg
ttttttttgccctagaattgaggaattccatttaccaagattctcttaatttgctcaaactcagtctcttcaccaatt
agtggataattatgtttggctaggatgcctagtccagctagattagattatgaagtatataccaaacaatgttcattt
atcaaattttcaattaacaatcaacgaaattttaaaatagaagctctacgaaaaatacatttaacatgagaaaccaac
atatattttaaaaactgttgataaacaataagaaaaaaacttgagacacagtttcgagttttttatttgacctgttga
ctttggtttcgcttaaggaccatagaaactgctaactcaggaaaaggaccaccttcgggcacaacggtcaggtgttcg
ggcagattggtctatgccatatcgcaacgatgacgtcaccaaattcatattttaaaactcgtttcgggcaacgacaac
gtcatgatccctcccaaaggtctaattgggccccggcccacaaaggttatcatcttctttcttcttctttgtttattg
ttgcttttgccttaaaatctcttcttcatcatcccaccgtttcttaagactctctctctttctgttttctatttctct
ctctctcaaatgaaagagagagaagagctcccatggatgaaattagcgagaccgaagtttctccaaggtgatatgtct
atctgtatatgtgatacgaagagttagggttttgtcatttcgaagtcaatttttgtttgtttgtcaataatgatatct
gaatgatgaagaacacgtaactaagatatgttactgaactatataatacatatgtgtgtttttctgtatctatttcta
tatatatgtagatgtagtgtaagtctgttatatagacattattcatgtgtacatgcattataccaacataaatttgta
tcaatactacttttgatttacgatgatggatgttcttagatatcttcatacgtttgtttccacatgtatttacaacta
catatatatttggaatcacatatatacttgattattatagttgtaaagagtaacaagttcttttttcaggcattaagg
aaaacataacctccgtgatgcatagagattattggatccgctgtgctgagacattgagtttttcttcggcattccagt
ttcaatgataaagcggtgttatcctatctgagcttttagtcggattttttcttttcaattattgtgttttatctagat
gatgcatttcattattctctttttcttgaccttgtaaggccttttcttgaccttgtaagaccccatctctttctaaac
gttttattattttctcgttttacagattctattctatctcttctcaatatagaatagatatcggcttcttcagcacct
tcaccttacgaattttctcctaccttgtctatccctcctgagctaatctccacatatatcttttgtttgttattgatg
tatggttgacataaattcaataaagaagttgacgtttttcttatttgatttttgttgttgttggttatattattgcaa
caaaattaaagggggtaaggaaggtctcgctatcaaggggactggcaaaaggtaatgaataaggaaacgggcaaaaag
aattatgcctttactctctcttttaaggctttggacaggaatttagttttttttatgtgttgtgttgtttgtttgggt
ctgactgaccccaaagggcaaagccaaaccagagaagactcttattaaatattccctcagaatcatttattgcctcta
tctttatctctctctttctcacactcgtgactgtttctcaccttatgtatgtactagtaatagtttttaccactttca
acttttacaaatagcatttgtttctgtttaaaacggttgacggataggtaatacccaaagttttatacaccaagaggc
gaggagttttaagtttataggtcgttcatagataaaaacaattgtttgtagacagaagtatatgtctctact-3'
2647nt
Number of nucleotides changed from the WT sequence: 18 (0.68%)
Tas3a (GEiGS ™) promoter region (SEQ ID NO: 64)
Number of nucleotides changed from the WT sequence: 0 (0%)
Tas3a (GEiGS ™) ncRNA (SEQ ID NO: 7):
5′-
atcccaccgtttcttaagactctctctctttctgttttctatttctctctctctcaaatgaaagagagagaagagctc
ccatggatgaaattagcgagaccgaagtttctccaaggtgatatgtctatctgtatatgtgatacgaagagttagggt
tttgtcatttcgaagtcaatttttgtttgtttgtcaataatgatatctgaatgatgaagaacacgtaactaagatatg
ttactgaactatataatacatatgtgtgtttttctgtatctatttctatatatatgtagatgtagtgtaagtctgtta
tatagacattattcatgtgtacatgcattataccaacataaatttgtatcaatactacttttgatttacgatgatgga
tgttcttagatatcttcatacgtttgtttccacatgtatttacaactacatatatatttggaatcacatatatacttg
attattatagttgtaaagagtaacaagttcttttttcaggcattaaggaaaacataacctccgtgatgcatagagatt
attggatccgctgtgctgagacattgagtttttcttcggcattccagtttcaatgataaagcggtgttatcctatctg
agcttttagtcggattttttcttttcaattattgtgttttatctagatgatgcatttcattattctctttttcttgac
cttgtaaggccttttcttgaccttgtaagaccccatctctttctaaacgttttattattttctcgttttacagattct
attctatctcttctcaatatagaatagatatcggcttcttcagcaccttcaccttacgaattttctcctaccttgtct
atccctcctgagctaatctccacatatatcttttgtttgttattgatgtatggttgacataaattcaataaagaagtt
gacgtttttct-3′
947nt
Number of nucleotides changed from the WT sequence: 18 (1.90%)
Tas3a (GEiGS ™) terminator region (SEQ ID NO: 65)
Number of nucleotides changed from the WT sequence: 0 (0%)
Primers for linear DNA preparation
Primers for blunt-end GEiGS ™ cassette production
Primers for PCR amplification of whole ath-miR173 GEiGS ™-Insertion example
cassette:
(SEQ ID NO: 11)
Primer Fw: 5′-cagaagccaaatctttggtaaacaaagtaatttttttg-3′
(SEQ ID NO: 12)
Primer Rev: 5′-aacttcaatattaaggtttcctgactaccctcac-3′
Primers for PCR amplification of whole ath-Tas3a GEiGS ™-Insertion example
cassette:
(SEQ ID NO: 13)
Primer Fw: 5′-atgaaattaagagccttgtcctagttttgtagtag-3′
(SEQ ID NO: 14)
Primer Fw: 5′-agtagagacatatacttctgtctacaaacaattgtttttatc-3′
Primers for sticky-end GEiGS ™ cassette production
Primers for PCR amplification of whole ath-miR173 GEiGS ™-Insertion example
cassette:
(SEQ ID NO: 15)
Primer Fw: 5′-nnxxxxxxcagaagccaaatctttggtaaacaaagtaatttttttg-3′
(SEQ ID NO: 16)
Primer Rev: 5′-nnxxxxxxaacttcaatattaaggtttcctgactaccctcac-3′
Primers for PCR amplification of whole ath-Tas3a GEiGS ™-Insertion example
cassette:
(SEQ ID NO: 17)
Primer Fw: 5′-nnxxxxxxatgaaattaagagccttgtcctagttttgtagtag-3′
(SEQ ID NO: 18)
Primer Rev: 5′-nnxxxxxxagtagagacatatacttctgtctacaaacaattgtttttatc-3′
Where:
- n: any nucleotide
-xxxxxx: restriction site for high-fidelity enzyme.
Primers for chemically-stabilised GEiGS ™ cassette production
Primers for PCR amplification of whole ath-miR173 GEiGS ™-Insertion example
cassette:
(SEQ ID NO: 19)
Primer Fw: 5′-c*agaagccaaatctttggtaaacaaagtaatttttttg-3′
(SEQ ID NO: 20)
Primer Rev: 5′-a*acttcaatattaaggtttcctgactaccctcac-3′
Primers for PCR amplification of whole ath-Tas3a GEiGS ™-Insertion example
cassette:
(SEQ ID NO: 21)
Primer Fw: 5′-a*tgaaattaagagccttgtcctagttttgtagtag-3′
(SEQ ID NO: 22)
Primer Rev: 5′-a*gtagagacatatacttctgtctacaaacaattgtttttatc-3′
Where: * represents a phosphorothioate linkage

### Example 3 Sequences

miR173 (GEiGS ™-Insertion) (SEQ ID NO: 62)
5′-
cagaagccaaatctttggtaaacaaagtaatttttttgccatttgattggttagtataggagaatttaaaaacgacga
taaggtttaggtaaattatttcatttgaaaataattgagcaccgttaataattttcatccataaaataatatttcaaa
gatgatatttgatccccattaaattcattcgtaaccaaaaaaaagttatgaaaaaagagtggtcgtgtgagttgccca
agcaccattataataaaaaataaaataattagcaagtaataaggaataaaatcctgtaattatagctgaaaaaggaaa
aatatttggagaccgtcagattcgaatctgaacaaagcataaaaaagtcaacaaaacttaaagcggcggtctcatcgt
aatctcagcccaataccctattttcctctcccctatataaatactttcttcttctactgatcttcttctcacaaataa
acccaaatatatcaatctactgtgttggtgattaagtactttgacaatccagccaatccagcagtggtcaaaaaagtt
gtagttttcttaaagtctctttcctctgctgattggcaggattgtcaaagagcttgctccctaaacttatctctctga
tgatttaatgttagagatcttcgtaaatctatgtgtttgatagatctgatgcgttttttgagttgatgatttgattat
ttttcactggaaagtatctcattagggtaacgataatgttttatggatttggttgtataacagatccatgaaatcttg
actggttataaaatctgaataatgtatttcaatttggagattcggtgataaaaattactgatttacgaatgacattta
tatcgatagatgagtttgctgatttggttgttaaattgataaatcaaggacatgagaaactgtttttgtatgctaatt
tgtccatggaataaaattgggaggtgaggaccgtgagggtagtcaggaaaccttaatattgaagtt-3′
1002nt
Number of nucleotides changed from the WT sequence: 31 (3.09%)
miR173 promoter region (SEQ ID NO: 60)
Number of nucleotides changed from the WT sequence: 0 (0%)
miR173 (GEIGSTM) ncRNA (SEQ ID NO: 2):
5′-
taagtactttgacaatccagccaatccagcagtggtcaaaaaagttgtagttttcttaaagtctctttcctctgctga
ttggcaggattgtcaaagagcttg-3′
102nt
Number of nucleotides changed from the WT sequence: 31 (30.39%)
miR173 terminator region (SEQ ID NO: 61)
Number of nucleotides changed from the WT sequence: 0 (0%)
Mutated ALS cassette
ALS precursor sequence (WT, nucleotide) (SEQ ID NO: 67)
ALS precursor sequence (P197S mutant, nucleotide) (SEQ ID NO: 68):
5′-
ATGGCGGCGGCAACAACAACAACAACAACATCTTCTTCGATCTCCTTCTCCACCA
AACCATCTCCTTCCTCCTCCAAATCACCATTACCAATCTCCAGATTCTCCCTCCCA
TTCTCCCTAAACCCCAACAAATCATCCTCCTCCTCCCGCCGCCGCGGTATCAAAT
CCAGCTCTCCCTCCTCCATCTCCGCCGTGCTCAACACAACCACCAATGTCACAAC
CACTCCCTCTCCAACCAAACCTACCAAACCCGAAACATTCATCTCCCGATTCGCT
CCAGATCAACCCCGCAAAGGCGCTGATATCCTCGTCGAAGCTTTAGAACGTCAA
GGCGTAGAAACCGTATTCGCTTACCCTGGAGGTGCATCAATGGAGATTCACCAA
GCCTTAACCCGCTCTTCCTCAATCCGTAACGTCCTTCCTCGTCACGAACAAGGAG
GTGTATTCGCAGCAGAAGGATACGCTCGATCCTCAGGTAAACCAGGTATCTGTAT
AGCCACTTCAGGTCCCGGAGCTACAAATCTCGTTAGCGGATTAGCCGATGCGTTG
TTAGATAGTGTTCCTCTTGTAGCAATCACAGGACAAGTCTCTCGTCGTATGATTG
GTACAGATGCGTTTCAAGAGACTCCGATTGTTGAGGTAACGCGTTCGATTACGAA
GCATAACTATCTTGTGATGGATGTTGAAGATATCCCTAGGATTATTGAGGAAGCT
TTCTTTTTAGCTACTTCTGGTAGACCTGGACCTGTTTTGGTTGATGTTCCTAAAGA
TATTCAACAACAGCTTGCGATTCCTAATTGGGAACAGGCTATGAGATTACCTGGT
TATATGTCTAGGATGCCTAAACCTCCGGAAGATTCTCATTTGGAGCAGATTGTTA
GGTTGATTTCTGAGTCTAAGAAGCCTGTGTTGTATGTTGGTGGTGGTTGTTTGAAT
TCTAGCGATGAATTGGGTAGGTTTGTTGAGCTTACGGGGATCCCTGTTGCGAGTA
CGTTGATGGGGCTGGGATCTTATCCTTGTGATGATGAGTTGTCGTTACATATGCTT
GGAATGCATGGGACTGTGTATGCAAATTACGCTGTGGAGCATAGTGATTTGTTGT
TGGCGTTTGGGGTAAGGTTTGATGATCGTGTCACGGGTAAGCTTGAGGCTTTTGC
TAGTAGGGCTAAGATTGTTCATATTGATATTGACTCGGCTGAGATTGGGAAGAAT
AAGACTCCTCATGTGTCTGTGTGTGGTGATGTTAAGCTGGCTTTGCAAGGGATGA
ATAAGGTTCTTGAGAACCGAGCGGAGGAGCTTAAGCTTGATTTTGGAGTTTGGAG
GAATGAGTTGAACGTACAGAAACAGAAGTTTCCGTTGAGCTTTAAGACGTTTGG
GGAAGCTATTCCTCCACAGTATGCGATTAAGGTCCTTGATGAGTTGACTGATGGA
AAAGCCATAATAAGTACTGGTGTCGGGCAACATCAAATGTGGGCGGCGCAGTTC
TACAATTACAAGAAACCAAGGCAGTGGCTATCATCAGGAGGCCTTGGAGCTATG
GGATTTGGACTTCCTGCTGCGATTGGAGCGTCTGTTGCTAACCCTGATGCGATAG
TTGTGGATATTGACGGAGATGGAAGCTTTATAATGAATGTGCAAGAGCTAGCCA
CTATTCGTGTAGAGAATCTTCCAGTGAAGGTACTTTTATTAAACAACCAGCATCT
TGGCATGGTTATGCAATGGGAAGATCGGTTCTACAAAGCTAACCGAGCTCACAC
ATTTCTCGGGGATCCGGCTCAGGAGGACGAGATATTCCCGAACATGTTGCTGTTT
GCAGCAGCTTGCGGGATTCCAGCGGCGAGGGTGACAAAGAAAGCAGATCTCCGA
GAAGCTATTCAGACAATGCTGGATACACCAGGACCTTACCTGTTGGATGTGATTT
GTCCGCACCAAGAACATGTGTTGCCGATGATCCCGAGTGGTGGCACTTTCAACGA
TGTCATAACGGAAGGAGATGGCCGGATTAAATACTGA-3′
2013nt
Number of nucleotides changed from the WT sequence: 1 (0.050%)
Putative chloroplast transit peptide (WT and P197S mutant, nucleotide)
(SEQ ID NO: 69)
Number of nucleotides changed from the WT sequence: 0 (0%)
ALS mature protein (WT, nucleotide) (SEQ ID NO: 70)
ALS mature protein (P197S mutant, nucleotide) (SEQ ID NO: 71):
5′-
AAAGGCGCTGATATCCTCGTCGAAGCTTTAGAACGTCAAGGCGTAGAAACCGTA
TTCGCTTACCCTGGAGGTGCATCAATGGAGATTCACCAAGCCTTAACCCGCTCTT
CCTCAATCCGTAACGTCCTTCCTCGTCACGAACAAGGAGGTGTATTCGCAGCAGA
AGGATACGCTCGATCCTCAGGTAAACCAGGTATCTGTATAGCCACTTCAGGTCCC
GGAGCTACAAATCTCGTTAGCGGATTAGCCGATGCGTTGTTAGATAGTGTTCCTC
TTGTAGCAATCACAGGACAAGTCTCTCGTCGTATGATTGGTACAGATGCGTTTCA
AGAGACTCCGATTGTTGAGGTAACGCGTTCGATTACGAAGCATAACTATCTTGTG
ATGGATGTTGAAGATATCCCTAGGATTATTGAGGAAGCTTTCTTTTTAGCTACTTC
TGGTAGACCTGGACCTGTTTTGGTTGATGTTCCTAAAGATATTCAACAACAGCTT
GCGATTCCTAATTGGGAACAGGCTATGAGATTACCTGGTTATATGTCTAGGATGC
CTAAACCTCCGGAAGATTCTCATTTGGAGCAGATTGTTAGGTTGATTTCTGAGTC
TAAGAAGCCTGTGTTGTATGTTGGTGGTGGTTGTTTGAATTCTAGCGATGAATTG
GGTAGGTTTGTTGAGCTTACGGGGATCCCTGTTGCGAGTACGTTGATGGGGCTGG
GATCTTATCCTTGTGATGATGAGTTGTCGTTACATATGCTTGGAATGCATGGGAC
TGTGTATGCAAATTACGCTGTGGAGCATAGTGATTTGTTGTTGGCGTTTGGGGTA
AGGTTTGATGATCGTGTCACGGGTAAGCTTGAGGCTTTTGCTAGTAGGGCTAAGA
TTGTTCATATTGATATTGACTCGGCTGAGATTGGGAAGAATAAGACTCCTCATGT
GTCTGTGTGTGGTGATGTTAAGCTGGCTTTGCAAGGGATGAATAAGGTTCTTGAG
AACCGAGCGGAGGAGCTTAAGCTTGATTTTGGAGTTTGGAGGAATGAGTTGAAC
GTACAGAAACAGAAGTTTCCGTTGAGCTTTAAGACGTTTGGGGAAGCTATTCCTC
CACAGTATGCGATTAAGGTCCTTGATGAGTTGACTGATGGAAAAGCCATAATAA
GTACTGGTGTCGGGCAACATCAAATGTGGGCGGCGCAGTTCTACAATTACAAGA
AACCAAGGCAGTGGCTATCATCAGGAGGCCTTGGAGCTATGGGATTTGGACTTC
CTGCTGCGATTGGAGCGTCTGTTGCTAACCCTGATGCGATAGTTGTGGATATTGA
CGGAGATGGAAGCTTTATAATGAATGTGCAAGAGCTAGCCACTATTCGTGTAGA
GAATCTTCCAGTGAAGGTACTTTTATTAAACAACCAGCATCTTGGCATGGTTATG
CAATGGGAAGATCGGTTCTACAAAGCTAACCGAGCTCACACATTTCTCGGGGAT
CCGGCTCAGGAGGACGAGATATTCCCGAACATGTTGCTGTTTGCAGCAGCTTGCG
GGATTCCAGCGGCGAGGGTGACAAAGAAAGCAGATCTCCGAGAAGCTATTCAGA
CAATGCTGGATACACCAGGACCTTACCTGTTGGATGTGATTTGTCCGCACCAAGA
ACATGTGTTGCCGATGATCCCGAGTGGTGGCACTTTCAACGATGTCATAACGGAA
GGAGATGGCCGGATTAAATAC-3′
1719nt
Number of nucleotides changed from the WT sequence: 0 (0.058%)
ALS precursor protein (WT, amino acid) (SEQ ID NO: 72)
ALS precursor protein (P197S mutant, amino acid) (SEQ ID NO: 73):
MAAATTTTTTSSSISFSTKPSPSSSKSPLPISRFSLPFSLNPNKSSSSSRRRGIKSSSPSSIS
AVLNTTTNVTTTPSPTKPTKPETFISRFAPDQPRKGADILVEALERQGVETVFAYPGG
ASMEIHQALTRSSSIRNVLPRHEQGGVFAAEGYARSSGKPGICIATSGPGATNLVSGL
ADALLDSVPLVAITGQVPRRMIGTDAFQETPIVEVTRSITKHNYLVMDVEDIPRIIEEA
FFLATSGRPGPVLVDVPKDIQQQLAIPNWEQAMRLPGYMSRMPKPPEDSHLEQIVRLI
SESKKPVLYVGGGCLNSSDELGRFVELTGIPVASTLMGLGSYPCDDELSLHMLGMH
GTVYANYAVEHSDLLLAFGVRFDDRVTGKLEAFASRAKIVHIDIDSAEIGKNKTPHV
SVCGDVKLALQGMNKVLENRAEELKLDFGVWRNELNVQKQKFPLSFKTFGEAIPPQ
YAIKVLDELTDGKAIISTGVGQHQMWAAQFYNYKKPRQWLSSGGLGAMGFGLPAAI
GASVANPDAIVVDIDGDGSFIMNVQELATIRVENLPVKVLLLNNQHLGMVMQWEDR
FYKANRAHTFLGDPAQEDEIFPNMLLFAAACGIPAARVTKKADLREAIQTMLDTPGP
YLLDVICPHQEHVLPMIPSGGTFNDVITEGDGRIKY
670aa
Number of amino acids changed from the WT sequence: 1 (0.149%)
Putative chloroplast transit peptide (WT and P197S mutant, amino acid)
(SEQ ID NO: 74)
Number of amino acids changed from the WT sequence: 0 (0%)
Mature ALS protein (WT, amino acid) (SEQ ID NO: 75)
573aa
Mature ALS protein (P197S mutant, amino acid) (SEQ ID NO: 76):
KGADILVEALERQGVETVFAYPGGASMEIHQALTRSSSIRNVLPRHEQGGVFAAEGY
ARSSGKPGICIATSGPGATNLVSGLADALLDSVPLVAITGQVPRRMIGTDAFQETPIVE
VTRSITKHNYLVMDVEDIPRIIEEAFFLATSGRPGPVLVDVPKDIQQQLAIPNWEQAM
RLPGYMSRMPKPPEDSHLEQIVRLISESKKPVLYVGGGCLNSSDELGRFVELTGIPVA
STLMGLGSYPCDDELSLHMLGMHGTVYANYAVEHSDLLLAFGVRFDDRVTGKLEA
FASRAKIVHIDIDSAEIGKNKTPHVSVCGDVKLALQGMNKVLENRAEELKLDFGVW
RNELNVQKQKFPLSFKTFGEAIPPQYAIKVLDELTDGKAIISTGVGQHQMWAAQFYN
YKKPRQWLSSGGLGAMGFGLPAAIGASVANPDAIVVDIDGDGSFIMNVQELATIRVE
NLPVKVLLLNNQHLGMVMQWEDRFYKANRAHTFLGDPAQEDEIFPNMLLFAAACG
IPAARVTKKADLREAIQTMLDTPGPYLLDVICPHQEHVLPMIPSGGTFNDVITEGDGR
IKY
573aa
Number of amino acids changed from the WT sequence: 0 (0.174%)
ALS promoter region (including 5′UTR) (SEQ ID NO: 77)
Number of nucleotides changed from the WT sequence: 0 (0%)
ALS terminator region (including 3′UTR) (SEQ ID NO: 78)
Number of nucleotides changed from the WT sequence: 0 (0%)
GEiGS ™-Insertion sequence with ALS selection (miR173 GEiGS ™ + P179S ALS)
(SEQ ID NO: 79)
5′-
CATATCAAGCTAGATTTTGAATTACCCTATTTCATCGTATACACAAAACTGATAA
TGTGGACACAGTTGATTTTACTTCTCGATGACATCGTAGTTTTATTTAATTTGGAA
ACCACGGCCCATATGAGCACATTTCAATTAAAAACCAATGGTAAGAGCATTTTCC
ATGCAAGATTCGAGAGATATTAACCCAGTGACTGTTAAAACAGCTTAGAACCCT
AATAACGAATTTCAATTACTCAATTTACCATTCGCATTTCGCAATAACCAAACTG
AGCCAGTCACAAGGAGTAAACCGAACCGGATTATTTATTTATAAAATGAAAGAA
AGGAAACCAAACAACAACAGCAGTAGTAGTCTGACGTAAACCAAAAAGCAGGC
AGATCAACAACTAAAAGAAACTCAAATTACCAAAACAAACAGGAAATTGCAAA
CTAAGTTTTTTTACCATATGCATACAAAGACCATAAAAGGTTCTGATAATCACCG
GTTTCATCTCTCAGTATTTAATCCGGCCATCTCCTTCCGTTATGACATCGTTGAAA
GTGCCACCACTCGGGATCATCGGCAACACATGTTCTTGGTGCGGACAAATCACAT
CCAACAGGTAAGGTCCTGGTGTATCCAGCATTGTCTGAATAGCTTCTCGGAGATC
TGCTTTCTTTGTCACCCTCGCCGCTGGAATCCCGCAAGCTGCTGCAAACAGCAAC
ATGTTCGGGAATATCTCGTCCTCCTGAGCCGGATCCCCGAGAAATGTGTGAGCTC
GGTTAGCTTTGTAGAACCGATCTTCCCATTGCATAACCATGCCAAGATGCTGGTT
GTTTAATAAAAGTACCTTCACTGGAAGATTCTCTACACGAATAGTGGCTAGCTCT
TGCACATTCATTATAAAGCTTCCATCTCCGTCAATATCCACAACTATCGCATCAG
GGTTAGCAACAGACGCTCCAATCGCAGCAGGAAGTCCAAATCCCATAGCTCCAA
GGCCTCCTGATGATAGCCACTGCCTTGGTTTCTTGTAATTGTAGAACTGCGCCGC
CCACATTTGATGTTGCCCGACACCAGTACTTATTATGGCTTTTCCATCAGTCAACT
CATCAAGGACCTTAATCGCATACTGTGGAGGAATAGCTTCCCCAAACGTCTTAAA
GCTCAACGGAAACTTCTGTTTCTGTACGTTCAACTCATTCCTCCAAACTCCAAAA
TCAAGCTTAAGCTCCTCCGCTCGGTTCTCAAGAACCTTATTCATCCCTTGCAAAG
CCAGCTTAACATCACCACACACAGACACATGAGGAGTCTTATTCTTCCCAATCTC
AGCCGAGTCAATATCAATATGAACAATCTTAGCCCTACTAGCAAAAGCCTCAAG
CTTACCCGTGACACGATCATCAAACCTTACCCCAAACGCCAACAACAAATCACTA
TGCTCCACAGCGTAATTTGCATACACAGTCCCATGCATTCCAAGCATATGTAACG
ACAACTCATCATCACAAGGATAAGATCCCAGCCCCATCAACGTACTCGCAACAG
GGATCCCCGTAAGCTCAACAAACCTACCCAATTCATCGCTAGAATTCAAACAACC
ACCACCAACATACAACACAGGCTTCTTAGACTCAGAAATCAACCTAACAATCTG
CTCCAAATGAGAATCTTCCGGAGGTTTAGGCATCCTAGACATATAACCAGGTAAT
CTCATAGCCTGTTCCCAATTAGGAATCGCAAGCTGTTGTTGAATATCTTTAGGAA
CATCAACCAAAACAGGTCCAGGTCTACCAGAAGTAGCTAAAAAGAAAGCTTCCT
CAATAATCCTAGGGATATCTTCAACATCCATCACAAGATAGTTATGCTTCGTAAT
CGAACGCGTTACCTCAACAATCGGAGTCTCTTGAAACGCATCTGTACCAATCATA
CGACGAGAGACTTGTCCTGTGATTGCTACAAGAGGAACACTATCTAACAACGCA
TCGGCTAATCCGCTAACGAGATTTGTAGCTCCGGGACCTGAAGTGGCTATACAGA
TACCTGGTTTACCTGAGGATCGAGCGTATCCTTCTGCTGCGAATACACCTCCTTGT
TCGTGACGAGGAAGGACGTTACGGATTGAGGAAGAGCGGGTTAAGGCTTGGTGA
ATCTCCATTGATGCACCTCCAGGGTAAGCGAATACGGTTTCTACGCCTTGACGTT
CTAAAGCTTCGACGAGGATATCAGCGCCTTTGCGGGGTTGATCTGGAGCGAATC
GGGAGATGAATGTTTCGGGTTTGGTAGGTTTGGTTGGAGAGGGAGTGGTTGTGAC
ATTGGTGGTTGTGTTGAGCACGGCGGAGATGGAGGAGGGAGAGCTGGATTTGAT
ACCGCGGCGGCGGGAGGAGGAGGATGATTTGTTGGGGTTTAGGGAGAATGGGAG
GGAGAATCTGGAGATTGGTAATGGTGATTTGGAGGAGGAAGGAGATGGTTTGGT
GGAGAAGGAGATCGAAGAAGATGTTGTTGTTGTTGTTGTTGCCGCCGCCATGGTT
CAGGAGAAAGATGAGCAAGAGATAGAGAAACAGAGAAGAAGAAGAGACTTGTG
AACATGATTTGTTTAAAAATGAGACTTGAAGTTTAAGGGTTTTATGTTGGATTAC
GAAAATGCCCTCGGGATTTGATTTTTGGTCCTTTATTTATAGATAAAACTCACGTG
ACGCTGGAATCTCTAGTCTATCTATTGTATTTGTGATTGAGCCTTCGATATGCTAA
AACCTAAATGTAGAATTTGTGGCTGAGAAATTTATCAAATTGATCTTTCTTTCTTT
TTTTATTGGTTAAGAGAATGGATACAAGCTGCAAGTTGGTTCCACGAATTGGACA
AAAACATAGTCCTGGTGATTTGAGATTTAGATTTTAAAGATACTTCTTGATGACA
ATATAATTTGTATACATGAATAAAAAACTTATAAGGTCTATAAGGAACAAGAGA
TAAGTGAAAATGGAATAAGAATGGAGAATTTGGCTACAAAAACAATAACAAAAC
TAAGAGCTTCCTCAAAGGGCTGATAATGTCGTTTGGTTCGTTCTTCTCCTCCTACT
CCTCGGAGCATTGCCTGTAGCGCGATTCAGAGTTTAGAAACTTTCACAAATGAGT
GTTTATCTCAGcagaagccaaatctttggtaaacaaagtaatttttttgccatttgattggttagtataggagaattt
aaaaacgacgataaggtttaggtaaattatttcatttgaaaataattgagcaccgttaataattttcatccataaaat
aatatttcaaagatgatatttgatccccattaaattcattcgtaaccaaaaaaaagttatgaaaaaagagtggtcgtg
tgagttgcccaagcaccattataataaaaaataaaataattagcaagtaataaggaataaaatcctgtaattatagct
gaaaaaggaaaaatatttggagaccgtcagattcgaatctgaacaaagcataaaaaagtcaacaaaacttaaagcggc
ggtctcatcgtaatctcagcccaataccctattttcctctcccctatataaatactttcttcttctactgatcttctt
ctcacaaataaacccaaatatatcaatctactgtgttggtgattaagtactttgacaatccagccaatccagcagtgg
tcaaaaaagttgtagttttcttaaagtctctttcctctgctgattggcaggattgtcaaagagcttgctccctaaact
tatctctctgatgatttaatgttagagatcttcgtaaatctatgtgtttgatagatctgatgcgttttttgagttgat
gatttgattatttttcactggaaagtatctcattagggtaacgataatgttttatggatttggttgtataacagatcc
atgaaatcttgactggttataaaatctgaataatgtatttcaatttggagattcggtgataaaaattactgatttacg
aatgacatttatatcgatagatgagtttgctgatttggttgttaaattgataaatcaaggacatgagaaactgttttt
gtatgctaatttgtccatggaataaaattgggaggtgaggaccgtgagggtagtcaggaaaccttaatattgaagtt-
3′
4135nt
Number of nucleotides changed from the WT sequence: 32 (0.774%)
miR173 GEiGS ™ expression cassette is shown in lowercase (transcribed from
the sense strand) and mutated ALS P197S cassette is shown in uppercase
(transcribed from the complementary strand). The whole GEiGS ™-Insertion
sequence was designed so that both cassettes are facing in opposite
directions.
Primers for GEiGS ™-Insertion cis-genic selection construct synthesis
Restriction strategy
Primers for PCR amplification of whole GEiGS ™-Insertion with selection
cassette:
(SEQ ID NO: 23)
Primer Fw: 5′-nnxxxxxxcatatcaagctagattttgaattaccctatttcatcgt-3′
(SEQ ID NO: 16)
Primer Rev: 5′-nnxxxxxxaacttcaatattaaggtttcctgactaccctcac-3′
Where:
- n: any nucleotide
-xxxxxx: restriction site for high-fidelity restriction enzyme.
Chemical modification strategy
Primers for PCR amplification of whole GEiGS ™-Insertion with selection
cassette:
(SEQ ID NO: 24)
Primer Fw: 5′-c*atatcaagctagattttgaattaccctatttcatcgt-3′
(SEQ ID NO: 20)
Primer Rev: 5′-a*acttcaatattaaggtttcctgactaccctcac-3′
Where:
A phosphorothioate bond is represented by *

### Example 4 Sequences

>GEiGS ™_insertion_At_miR173_to_PDS3_ALS1
(SEQ ID NO: 79)
>At_GEiGS ™_geno_F1
(SEQ ID NO: 25)
5′-acaaactacctgcaagtggtca-3′
>At_GEiGS ™_geno_R1
(SEQ ID NO: 26)
5′-caagctctttatccacact-3′
>Tomato GEiGS ™-Insertion construct
(SEQ ID NO: 80)
>TomGEiGS ™_linear_F1
(SEQ ID NO: 27)
5′-A*ATTCAAAGCCAATGGAACT-3′
Note - asterisk signifies phosphorothioate
linkage
>TomGEiGS ™_linear_R1
(SEQ ID NO: 28)
5′-A*GTACAAGATGTCAAATGCC-3′
Note - asterisk signifies phosphorothioate
linkage
>S1_GEiGS ™_geno_F1
(SEQ ID NO: 29)
5′-GGTAACTCATTTGGGATGGTA-3′
>S1_GEiGS ™_geno_R1
(SEQ ID NO: 30)
5'-GAACGGTCGCTACGATTAC-3'

### Example 5 Sequences

>Wild_type_Arabidopsis_thaliana_miR173
(SEQ ID NO: 1)
5′-
taagtactttcgcttgcagagagaaatcacagtggtcaaaaaagttgtag
ttttcttaaagtctctttcctctgtgattctctgtgtaagcgaaagagct
tg-3′
>Arabidopsis_thaliana_miR173_modified_to_target_
PDS3
(SEQ ID NO: 2)
5′-
taagtactttgacaatccagccaatccagcagtggtcaaaaaagttgtag
ttttcttaaagtctctttcctctgctgattggcaggattgtcaaagagct
tg-3′
>Mature_siRNA_from_modified_AtmiR173
(SEQ ID NO: 3)
5′-tgacaatccagccaatccagc-3′
>Arabidopsis_thaliana_pytoene_desaturase_3_
(PDS3)_transcript (SEQ ID NO: 81)
>GEiGS ™_Insertion_At_MiR173_to_PDS3_ALS1
(SEQ ID NO: 79)
>GEiGS ™_dummy_At_miR173_wt_ALS1
(SEQ ID NO: 82)
>At_GEiGS ™_geno_F2
(SEQ ID NO: 31)
5′-AATCCAGCAGTGGTCAAAA-3′
>At_GEiGS ™_geno_R2
(SEQ ID NO: 32)
5′-CAAGCTCTTTGACAATCCT-3′
>At_GEiGS ™_dummy_geno_F1
(SEQ ID NO: 33)
5′-GAGCTTCCTCAAAGGGCTGA-3′
>At_GEiGS ™_dummy_geno_R1
(SEQ ID NO: 34)
5′-TGGTTACGAATGAATTTAATGGGGA-3′
>AC1
(SEQ ID NO: 35)
5′-ACGATGGACTCCAGAG-3′
>LAD1-1
(SEQ ID NO: 36)
5′-ACGATGGACTCCAGAGCGGCCGC(G/C/A)N(G/C/A)
NNNGGAA-3′
>LAD1-2
(SEQ ID NO: 37)
5′-ACGATGGACTCCAGAGCGGCCGC(G/C/T)N(G/C/T)
NNNGGTT-3′
>LAD1-3
(SEQ ID NO: 38)
5′-ACGATGGACTCCAGAGCGGCCGC(G/C/A)(G/C/A)N
(G/C/A)NNNCCAA-3′
>LAD1-4
(SEQ ID NO: 39)
5′-ACGATGGACTCCAGAGCGGCCGC(G/C/T)(G/A/T)N
(G/C/T)NNNCGGT-3′
>At_GEiGS ™_TAIL_F1
(SEQ ID NO: 40)
TAAGTACTTTGACAATCCAGCCAATCCAGCAGTGGT
>At_GEiGS ™_TAIL_F2
(SEQ ID NO: 41)
GACAATCCAGCCAATCCAGCAGTGGTCAAAAAAG
>At_GEiGS ™_TAIL_F3
(SEQ ID NO: 42)
CTCTGCTGATTGGCAGGATTGTCAAAGAGCTTGCTC

### Example 6 Sequences

>TuMV8 (1.2 kb)
(SEQ ID NO: 83)
AGGATAGAAAATTTCAGATGATGGTGAAAACTTAGAGGAGTAGTTCTCTGGTAT
AGTGACCAATGCATAAGTCGAATCATTTGGGTAATCTAGTTCTGAAATCTTAGGA
TGCCATATGCGGTGGTAATATGTGATGTTTCCGGGTACGGAAATTTCTTTGGAAC
TAAGAATTAGAGTGAGTGAAGTCACATGAACATTAATGTAGATCACTGATCATCT
GGTGGATATCAAAACAATCAACCACAAAACAAGAAAACAAATGTACGTAGAATT
CCAAGACCATTAGTGATTACGATATATTAAGCTTACATTGCATCTTAGAAGGCAA
TTCAAGGTCATCATGCATGTTCAAAGAAGATAAACAATGATCCTTTACCTCTTGT
GTGAAGCAAAGTTTGTTTAACAATTCTCTAGGCATCTCTAATGGAATAAAACTAA
GAGGACCACCGAGTTCAGTTTCCAGAAGCTTCGTACGTAGCTTTCTCCACGaatcaga
tggtgttaagtatcaacttcgcaccaagagaggccagccttctATTATTTCAAAGTAGTTGCTG
cacgtatactccacgagatgctgtttcgaaaaacaatagagacctctaacttcggaacggcaga
caaaggagtagacacacggaggctagtttacatagttgaagggaagcctctcaaaggcaca
ctgaaggtacttaaaaccatatgatACCAAGGAGAAATCAATGAAACTT
TTAGAGACTGAATGATGTTGTTATCTTAGTTGTCTTCCACTAGAGATGCATAATG
GAATCGTTAATAAAATTTGCCCCGCACAAAACGAAGGATTATTGCTCTTATGCTT
TAAACATGTATATAATATTACACTACCTTTTAAGATGCGAGGTAAGCTAAATAAA
TACTAGTGATTAAGAAATATTACAACTGTCTTCTTCTTGCGTTGCTACTTGATTCG
ACCACATGATCGCTACAAGGATGTTCACTCAATCTATTTGTTGATGACGAAGTAT
CTTCCTCACCAGATGATGGGGTCATCAAGTCAGCATCAACCAAGGATCATGGAG
GAGGCACCAATAAGTCGACCAAGATCCACGAGAGAACCAACCTAAGCATACCAA
ATACATATGTGCCTTTCAAGAAGCATACCAGCCCAACCCGAAGAATTTAAGCTAC
AAATCAACTTATTCCAAAAACTTAACT
>Dummy17 (1.2 kb)
(SEQ ID NO: 84)
AGGATAGAAAATTTCAGATGATGGTGAAAACTTAGAGGAGTAGTTCTCTGGTAT
AGTGACCAATGCATAAGTCGAATCATTTGGGTAATCTAGTTCTGAAATCTTAGGA
TGCCATATGCGGTGGTAATATGTGATGTTTCCGGGTACGGAAATTTCTTTGGAAC
TAAGAATTAGAGTGAGTGAAGTCACATGAACATTAATGTAGATCACTGATCATCT
GGTGGATATCAAAACAATCAACCACAAAACAAGAAAACAAATGTACGTAGAATT
CCAAGACCATTAGTGATTACGATATATTAAGCTTACATTGCATCTTAGAAGGCAA
TTCAAGGTCATCATGCATGTTCAAAGAAGATAAACAATGATCCTTTACCTCTTGT
GTGAAGCAAAGTTTGTTTAACAATTCTCTAGGCATCTCTAATGGAATAAAACTAA
GAGGACCACCGAGTTCAGTTTCCAGAAGCTTCGTACGTAGCTTTCTCCACGaattggg
aggcggtaggtacggatttctcaccgatggtaaccagacagcTTTCGTGGGGTTCACTAATCTg
cacgtacactctacgaaattcttttgcggtaagcaacagggacacctgacaccgcaaaggaaga
caaaagagtggacatacggtttagtcggcggatgagtgttgtgaagttgccaaaaggcacaaa
gaaagtacctacagcctcacgatACCAAGGAGAAATCAATGAAAC
TTTTAGAGACTGAATGATGTTGTTATCTTAGTTGTCTTCCACTAGAGATGCATAAT
GGAATCGTTAATAAAATTTGCCCCGCACAAAACGAAGGATTATTGCTCTTATGCT
TTAAACATGTATATAATATTACACTACCTTTTAAGATGCGAGGTAAGCTAAATAA
ATACTAGTGATTAAGAAATATTACAACTGTCTTCTTCTTGCGTTGCTACTTGATTC
GACCACATGATCGCTACAAGGATGTTCACTCAATCTATTTGTTGATGACGAAGTA
TCTTCCTCACCAGATGATGGGGTCATCAAGTCAGCATCAACCAAGGATCATGGA
GGAGGCACCAATAAGTCGACCAAGATCCACGAGAGAACCAACCTAAGCATACCA
AATACATATGTGCCTTTCAAGAAGCATACCAGCCCAACCCGAAGAATTTAAGCT
ACAAATCAACTTATTCCAAAAACTTAACT
>TuMV10 (1.2 kb)
(SEQ ID NO: 85)
GCCAACTTCTATCTCACCTACAAATGTATAAAGGGCATTGTTACAAGAACACAGG
AAAATTTGTACCGATTTATACAATGGCAAATTTGTACCGATTTGTACCTACAAAT
GTACCGATTTATAAAGAGAAATCTTGCATGTTGGAAAAAAGCAAGAAAATTTTG
AAGCTAGACTTTTTGATACATGAAACAAAACACAACACTTTTTCAGGAACATTGT
TTGGTCAGACGAAGTAAGCAACCTACAAGAATCTGAACTTCTTTTTAATTATCCA
ACGCCAAATCTACTTGATAAACATTAAGATAAAGCTAAGGCGCTTATCAGTCAA
CTACACACTTCAATAACAAGCACTTCATTAACTGAAAAAACAAACAAACTAAAG
AGGTCGAATTCTCACCAGAAACAACAGTCACCTCTTCATCTGCTCTCTAAGAACA
CAAAATCCTCAGCTTTCCCAGAAAAGTAATCGGACTTGACTCTGTTTTCCCTCAC
TTTTTTCTTCTTTCAAAAACCAATAAACAAAAAGATTCcagaaactgagaacgatacccggggt
cgtcgcttcagcgctaatattgtagaccttttcttgcttttacctctttgaagtgttcacagga
agaAGCCTACTACATGAACAATAAagcggcccaacctcttctcgcttcttAACTCCAAAATGTC
AACGAAACATAAATTTTCCGAATCGGATCAAACGAAACACATCAGATCTGAAAATTCAACAGTC
AAAATCTATACCACAAGTAGTAAATAGCTGGATCTTTATTGCCTTATGAAACCAAATGGACAAA
AACATATACCCTAGATTACACACATGCATCTGCAGACAAACTGGGAGAAAGAGAA
TCAGTGTGATGATAGAGATATGTCAAATTAATGAATCCAACAAAGAAAGAAAAA
GGAAACAGAGAGATGAAAGTTTTAACCAGGAATCTTCTTGACCAGATACCATTT
GTGATTCTCTGATTTGCCTATTGGCCTACTACATCCCTCCTCTCTCTCTATCTTTTA
ATAACAAACTTTATAGATCTGGTTTTTTCTTCTAGAACTAAAATGTATTTGATTCA
TATTTATAACTAAAATGTATCTAAAAGTCTCATAACATATTTGTCCAAGAAAAAA
TTGTAGTAACATAGTTATTGAATTTGAATATCATAAGATTTAGGAAGACATTTTA
AA
>Dummy19 (1.2 kb)
(SEQ ID NO: 86)
GCCAACTTCTATCTCACCTACAAATGTATAAAGGGCATTGTTACAAGAACACAGG
AAAATTTGTACCGATTTATACAATGGCAAATTTGTACCGATTTGTACCTACAAAT
GTACCGATTTATAAAGAGAAATCTTGCATGTTGGAAAAAAGCAAGAAAATTTTG
AAGCTAGACTTTTTGATACATGAAACAAAACACAACACTTTTTCAGGAACATTGT
TTGGTCAGACGAAGTAAGCAACCTACAAGAATCTGAACTTCTTTTTAATTATCCA
ACGCCAAATCTACTTGATAAACATTAAGATAAAGCTAAGGCGCTTATCAGTCAA
CTACACACTTCAATAACAAGCACTTCATTAACTGAAAAAACAAACAAACTAAAG
AGGTCGAATTCTCACCAGAAACAACAGTCACCTCTTCATCTGCTCTCTAAGAACA
CAAAATCCTCAGCTTTCCCAGAAAAGTAATCGGACTTGACTCTGTTTTCCCTCAC
TTTTTTCTTCTTTCAAAAACCAATAAACAAAAAGATTCcagaaaatacgtcagatacgcgaggc
ttacgcttccttgggccccattaacctcagctcttgcttttacaattttattgtgttcgcagga
ggcgAGATTAGTGAACCCCACGAAAgcgactcactctcaacgtccttcttAACTCCAAAATGTC
AAACGAAACATAAATTTTCCGATCGGATCAAACGAAACACATCAGATCTGAAAATTCAACAGTC
AAAATCTATACCACAAGTAGTAAATAGCTGGATCTTTATTGCCTTATGAAACCAAATGGACAAA
AACATATACCCTAGATTACACACATGCATCTGCAGACAAACTGGGAGAAAGAGAA
TCAGTGTGATGATAGAGATATGTCAAATTAATGAATCCAACAAAGAAAGAAAAA
GGAAACAGAGAGATGAAAGTTTTAACCAGGAATCTTCTTGACCAGATACCATTT
GTGATTCTCTGATTTGCCTATTGGCCTACTACATCCCTCCTCTCTCTCTATCTTTTA
ATAACAAACTTTATAGATCTGGTTTTTTCTTCTAGAACTAAAATGTATTTGATTCA
TATTTATAACTAAAATGTATCTAAAAGTCTCATAACATATTTGTCCAAGAAAAAA
TTGTAGTAACATAGTTATTGAATTTGAATATCATAAGATTTAGGAAGACATTTTA
AA
>TuMV12 (1.2 kb)
(SEQ ID NO: 87)
GTAAAAAAACATGTGACGACGAAATTCAATCAGTTCCTCCAATGTTTGGCAGAA
GCCAAATCTTTGGTAAACAAAGTAATTTTTTTGCCATTTGATTGGTTAGTATAGG
AGAATTTAAAAACGACGATAAGGTTTAGGTAAATTATTTCATTTGAAAATAATTG
AGCACCGTTAATAATTTTCATCCATAAAATAATATTTCAAAGATGATATTTGATC
CCCATTAAATTCATTCGTAACCAAAAAAAAGTTATGAAAAAAGAGTGGTCGTGT
GAGTTGCCCAAGCACCATTATAATAAAAAATAAAATAATTAGCAAGTAATAAGG
AATAAAATCCTGTAATTATAGCTGAAAAAGGAAAAATATTTGGAGACCGTCAGA
TTCGAATCTGAACAAAGCATAAAAAAGTCAACAAAACTTAAAGCGGCGGTCTCA
TCGTAATCTCAGCCCAATACCCTATTTTCCTCTCCCCTATATAAATACTTTCTTCTT
CTACTGATCTTCTTCTCACAAATAAACCCAAATATATCAATCTACTGTGTTGGTG
ATaaagcacttTACATGTTGATGAACTTCCTGagcggtcaaaaaagtggcgaaatatctcaagc
aaggttccacttaggggttcagcggtatgtaaagagctttCTCCCTAAACTTATCTCTCTGATG
ATTTAATGTTAGAGATCTTCGTAAATCTATGTGTTTGATAGATCTGATGCGTTTTTTGAGTTGA
TGATTTGATTATTTTTCACTGGAAAGTATCTCATTAGGGTAACGATAATGTTTTATGGATTT
GGTTGTATAACAGATCCATGAAATCTTGACTGGTTATAAAATCTGAATAATGTAT
TTCAATTTGGAGATTCGGTGATAAAAATTACTGATTTACGAATGACATTTATATC
GATAGATGAGTTTGCTGATTTGGTTGTTAAATTGATAAATCAAGGACATGAGAAA
CTGTTTTTGTATGCTAATTTGTCCATGGAATAAAATTGGGAGGTGAGGACCGTGA
GGGTAGTCAGGAAACCTTAATATTGAAGTTGATGTTGAACCAACAAATCTGCCC
AAAAATGATAAAAGTTGATGCCGAGCCCACAAATTTTGATGACAATCGATAAGC
CCAAGCCCAAAAGGCATCTGTACCTGAGCCCATTATTCTTTCATTACTAGCAAAA
AGGATGCATTAGAGAC
>TuMV14 (1.2 kb)
(SEQ ID NO: 88)
GTAAAAAAACATGTGACGACGAAATTCAATCAGTTCCTCCAATGTTTGGCAGAA
GCCAAATCTTTGGTAAACAAAGTAATTTTTTTGCCATTTGATTGGTTAGTATAGG
AGAATTTAAAAACGACGATAAGGTTTAGGTAAATTATTTCATTTGAAAATAATTG
AGCACCGTTAATAATTTTCATCCATAAAATAATATTTCAAAGATGATATTTGATC
CCCATTAAATTCATTCGTAACCAAAAAAAAGTTATGAAAAAAGAGTGGTCGTGT
GAGTTGCCCAAGCACCATTATAATAAAAAATAAAATAATTAGCAAGTAATAAGG
AATAAAATCCTGTAATTATAGCTGAAAAAGGAAAAATATTTGGAGACCGTCAGA
TTCGAATCTGAACAAAGCATAAAAAAGTCAACAAAACTTAAAGCGGCGGTCTCA
TCGTAATCTCAGCCCAATACCCTATTTTCCTCTCCCCTATATAAATACTTTCTTCTT
CTACTGATCTTCTTCTCACAAATAAACCCAAATATATCAATCTACTGTGTTGGTG
ATaaggaacatTTATTGTTCATGTAGTAGGCTcggggtcaaaaaagtcggaggatgcttgaagt
cgcaatccgcgggccactacagggatagtgaatgatccttCTCCCTAAACTTATCTCTCTGATG
ATTTAATGTTAGAGATCTTCGTAAATCTATGTGTTTGATAGATCTGATGCGTTTTTTGAGTTGA
TGATTTGATTATTTTTCACTGGAAAGTATCTCATTAGGGTAACGATAATGTTTTATGGATT
TGGTTGTATAACAGATCCATGAAATCTTGACTGGTTATAAAATCTGAATAATGTA
TTTCAATTTGGAGATTCGGTGATAAAAATTACTGATTTACGAATGACATTTATAT
CGATAGATGAGTTTGCTGATTTGGTTGTTAAATTGATAAATCAAGGACATGAGAA
ACTGTTTTTGTATGCTAATTTGTCCATGGAATAAAATTGGGAGGTGAGGACCGTG
AGGGTAGTCAGGAAACCTTAATATTGAAGTTGATGTTGAACCAACAAATCTGCC
CAAAAATGATAAAAGTTGATGCCGAGCCCACAAATTTTGATGACAATCGATAAG
CCCAAGCCCAAAAGGCATCTGTACCTGAGCCCATTATTCTTTCATTACTAGCAAA
AAGGATGCATTAGAGAC
>Dummy21
(SEQ ID NO: 89)
GTAAAAAAACATGTGACGACGAAATTCAATCAGTTCCTCCAATGTTTGGCAGAA
GCCAAATCTTTGGTAAACAAAGTAATTTTTTTGCCATTTGATTGGTTAGTATAGG
AGAATTTAAAAACGACGATAAGGTTTAGGTAAATTATTTCATTTGAAAATAATTG
AGCACCGTTAATAATTTTCATCCATAAAATAATATTTCAAAGATGATATTTGATC
CCCATTAAATTCATTCGTAACCAAAAAAAAGTTATGAAAAAAGAGTGGTCGTGT
GAGTTGCCCAAGCACCATTATAATAAAAAATAAAATAATTAGCAAGTAATAAGG
AATAAAATCCTGTAATTATAGCTGAAAAAGGAAAAATATTTGGAGACCGTCAGA
TTCGAATCTGAACAAAGCATAAAAAAGTCAACAAAACTTAAAGCGGCGGTCTCA
TCGTAATCTCAGCCCAATACCCTATTTTCCTCTCCCCTATATAAATACTTTCTTCTT
CTACTGATCTTCTTCTCACAAATAAACCCAAATATATCAATCTACTGTGTTGGTG
ATaaagcactTTTCGTGGGGTTCACTAATCTctgcggacaaaaaagattcacatcggtaatacc
cacaagccccagagatagtggtttccacggaaagagctttCTCCCTAAACTTATCTCTCTGATG
ATTTAATGTTAGAGATCTTCGTAAATCTATGTGTTTGATAGATCTGATGCGTTTTTTGAGTTGA
TGATTTGATTATTTTTCACTGGAAAGTATCTCATTAGGGTAACGATAATGTTTTATGGATT
TGGTTGTATAACAGATCCATGAAATCTTGACTGGTTATAAAATCTGAATAATGTA
TTTCAATTTGGAGATTCGGTGATAAAAATTACTGATTTACGAATGACATTTATAT
CGATAGATGAGTTTGCTGATTTGGTTGTTAAATTGATAAATCAAGGACATGAGAA
ACTGTTTTTGTATGCTAATTTGTCCATGGAATAAAATTGGGAGGTGAGGACCGTG
AGGGTAGTCAGGAAACCTTAATATTGAAGTTGATGTTGAACCAACAAATCTGCC
CAAAAATGATAAAAGTTGATGCCGAGCCCACAAATTTTGATGACAATCGATAAG
CCCAAGCCCAAAAGGCATCTGTACCTGAGCCCATTATTCTTTCATTACTAGCAAA
AAGGATGCATTAGAGAC

1. A method of reducing the expression of a first target gene comprising inserting a silencing insertion into the genome of a eukaryotic cell,
wherein the silencing insertion comprises a promoter or a part thereof, a sequence encoding a silencing RNA that silences the expression of the first target gene, and a terminator or a part thereof,
wherein the silencing insertion has over 95% sequence identity across its length to an endogenous silencing sequence in the eukaryotic cell that comprises a promoter, a sequence encoding a silencing RNA and a terminator, and
wherein the silencing RNA encoded by the endogenous silencing sequence is not active or silences the expression of a second target gene that is different from the first target gene.

2. The method of claim 1, wherein the silencing insertion is inserted by particle bombardment, protoplast transfection, electroporation, nanoparticle-mediated transfection or Agrobacterium-mediated transformation.

3. A eukaryotic cell comprising in its genome:
(a) a silencing insertion comprising a promoter or a part thereof, a sequence encoding a silencing RNA that silences the expression of a first target gene, and a terminator or a part thereof,
(b) an endogenous genome sequence that has been separated by the insertion, and
(c) an endogenous silencing sequence comprising a promoter, a sequence encoding a silencing RNA and a terminator,
wherein the silencing insertion and the endogenous silencing sequence have over 95% sequence identity across the length of the insertion, and
wherein the silencing RNA encoded by the endogenous silencing sequence is not active or silences the expression of a second target gene that is different from the first target gene.

4. The eukaryotic cell of claim 3, wherein an endogenous genome sequence has been deleted from between the endogenous genome sequence that has been separated.

5. An isolated silencing construct comprising a silencing insertion that comprises a promoter or a part thereof, a sequence encoding a silencing RNA that silences the expression of a first target gene in a eukaryotic cell, and a terminator or a part thereof,
wherein the silencing insertion has over 95% sequence identity across its length to an endogenous silencing sequence in the eukaryotic cell that comprises a promoter, a sequence encoding a silencing RNA and a terminator, and
wherein the silencing RNA encoded by the endogenous silencing sequence is not active or silences the expression of a second target gene that is different from the first target gene.

6. The method, eukaryotic cell or isolated silencing construct of any preceding claim, wherein said eukaryotic cell is a plant cell, such as a banana plant cell, a coffee plant cell or a rice plant cell.

7. The method or eukaryotic cell of claim 6, wherein the eukaryotic cell is a plant protoplast.

8. The method, eukaryotic cell or isolated silencing construct of any of claims 1-5, wherein said eukaryotic cell is a human cell or an animal cell, preferably a livestock animal cell, such as a horse, donkey, cattle or any other ruminant, zebu, yak, buffalo, sheep, goat, reindeer, camel, llama, alpaca, pig, rabbit or any other rodent, dog, cat, poultry such as chicken, turkey, goose or duck, fish, crustacean or mollusc cell.

9. The method, eukaryotic cell or isolated silencing construct of any preceding claim, wherein the silencing RNA encoded by the silencing insertion is a small interfering RNA (siRNA), a microRNA (miRNA), a piwi-interacting RNA (piRNA), a phased small interfering RNA (phasiRNA), a trans-acting siRNA (tasiRNA), a short-hairpin RNA (shRNA), inverted repeat RNA forming double stranded RNA, a small nuclear RNA (snRNA or U-RNA), a small nucleolar RNA (snoRNA), a Small Cajal body RNA (scaRNA), a transfer RNA (tRNA), a ribosomal RNA (rRNA), a repeat-derived RNA, an autonomous or non-autonomous transposable or retro-transposable element-derived RNA, an autonomous or non-autonomous transposable or retro-transposable element RNA, a transfer RNA fragment (tRF), or a long non-coding RNA (lncRNA).

10. The method, eukaryotic cell or isolated silencing construct of any preceding claim, wherein the silencing insertion comprises 1-40, such as 5-40, 5-30, 5-20, 3-40, 3-30, 3-20, 5-15, 3-10, 10-30, or 10-20 nucleotide substitutions, additions and/or deletions relative to the endogenous silencing sequence.

11. The method, eukaryotic cell or isolated silencing construct of claim 10, wherein substitutions are not contiguous.

12. The method, eukaryotic cell or isolated silencing construct of claim 10 or claim 11, wherein the nucleotide substitutions are all present in the sequence encoding the silencing RNA.

13. The method, eukaryotic cell or isolated silencing construct of claim 10 or claim 11, wherein the nucleotide substitutions are all present in the transcribed sequence.

14. The method, eukaryotic cell or isolated silencing construct of any preceding claim, wherein the silencing RNA encoded by the silencing insertion and the silencing RNA encoded by the endogenous silencing sequence form the same secondary structure.

15. The method, eukaryotic cell or isolated silencing construct of any preceding claim, wherein the promoter and terminator in the silencing insertion are identical or partly identical to the promoter and terminator in the endogenous silencing sequence.

16. The method, eukaryotic cell or isolated silencing construct of any preceding claim, wherein the silencing insertion and the endogenous silencing sequence have over 96%, 97%, 98%, 99%, 99.5%, 99.6%, 99.7%, 99.8%, or 99.9% sequence identity across the length of the insertion.

17. The method of any preceding claim, wherein the silencing insertion is inserted with an adjacent selectable marker, or the eukaryotic cell or isolated silencing construct of any preceding claim, wherein the silencing insertion is adjacent to a selectable marker.

18. The method, eukaryotic cell or isolated silencing construct of claim 17, wherein the selectable marker is endogenous to the eukaryotic cell.

19. The method, eukaryotic cell or isolated silencing construct of claim 18, wherein the selectable marker is a mutant version of a marker endogenous to the eukaryotic cell or wherein the selectable marker is a mutant version of a gene endogenous to the eukaryotic cell which does not function as a selectable marker.

20. The method, eukaryotic cell or isolated silencing construct of claim 19, wherein the mutation in the mutant version is the same as a naturally occurring mutation in another eukaryotic cell of the same genus, optionally of the same species.

21. The method, eukaryotic cell or isolated silencing construct of claim 19 or 20, wherein the selectable marker is a mutated version of an ALS gene that confers herbicide resistance, such as chlorsulfuron herbicide resistance.

22. The method of any of claims 1, 2 and 6-21, wherein the silencing insertion is inserted into the genome of the eukaryotic cell in a construct that additionally comprises sequence encoding an RNA-guided DNA endonuclease and sequence encoding a guide RNA targeted to the endogenous silencing sequence.

23. The method of claim 22, wherein the RNA-guided DNA endonuclease and guide RNA introduce a modification into the endogenous silencing sequence, such as an insertion, deletion or insertion and deletion, which reduces or abolishes expression or activity of the endogenous silencing sequence.

24. The method of claim 22, wherein the RNA-guided DNA endonuclease and guide RNA mediate substitution of the endogenous silencing sequence with the silencing insertion in the eukaryotic cell.

25. The method of claim 22, wherein the eukaryotic cell is a plant cell or a livestock animal cell, and wherein the method additionally comprises generating a plant or a livestock animal and breeding the plant or livestock animal for at least one generation until the RNA-guided DNA endonuclease and guide RNA mediate substitution of the endogenous silencing sequence with the silencing insertion.

26. The method claim 24 or claim 25, wherein the eukaryotic cell is a plant cell or a livestock animal cell and wherein the method additionally comprises generating a plant or a livestock animal and breeding out the construct that was inserted into the genome, thereby generating a plant or livestock animal that comprises the silencing insertion substituted at the location of the endogenous silencing sequence and that does not comprise the construct that was inserted into the genome of the eukaryotic cell.

27. The eukaryotic cell of any of claims 3, 4 and 6-21, wherein the eukaryotic cell comprises in its genome a construct that comprises sequence encoding an RNA-guided DNA endonuclease, sequence encoding a guide RNA targeted to the endogenous silencing sequence, and a silencing insertion as defined in any preceding claim.

28. The eukaryotic cell of claim 27, wherein the endogenous silencing sequence comprises a modification introduced by the RNA-guided DNA endonuclease and guide RNA, such as an insertion, deletion or insertion and deletion, which reduces or abolishes expression or activity of the endogenous silencing sequence.

29. The silencing construct of any of claims 5-21, additionally comprising sequence encoding an RNA-guided DNA endonuclease and sequence encoding a guide RNA targeted to the endogenous silencing sequence.

30. The method of any of claims 1 and 6-26, wherein the silencing insertion is introduced into the eukaryotic cell in a T-DNA cassette using Agrobacterium-mediated transformation, or the eukaryotic cell of any of claims 3, 4, 6-21, 27 and 28 wherein the silencing insertion is within an inserted T-DNA cassette sequence, or the isolated silencing construct of any of claims 5-21 and 29, which is a T-DNA cassette.

31. The method, eukaryotic cell or silencing insertion of claim 30, wherein the T-DNA cassette comprises a selectable marker and/or sequence encoding an RNA-guided DNA endonuclease and sequence encoding a guide RNA targeted to the endogenous silencing sequence.

32. The method, eukaryotic cell or isolated silencing construct of any preceding claim, wherein the method comprises inserting multiple silencing insertions, wherein the eukaryotic cell comprises multiple silencing insertions, or wherein the isolated silencing construct comprises multiple silencing insertions.

33. The method, eukaryotic cell or isolated silencing construct of claim 32, wherein the silencing RNA encoded by each silencing insertion targets a different gene.

34. The method, eukaryotic cell or isolated silencing construct of any preceding claim, wherein the target gene is endogenous to the eukaryotic cell.

35. The method, eukaryotic cell or isolated silencing construct of claim 34, wherein the silencing of the target gene provides an improved trait or phenotype.

36. The method, eukaryotic cell or isolated silencing construct of any of claims 1-33, wherein the target gene is exogenous to the eukaryotic cell, such as a pest gene.

37. The method, eukaryotic cell or isolated silencing construct of claim 36, wherein silencing of the target gene provides pest resistance, disease tolerance or resistance.

38. The method, eukaryotic cell or isolated silencing construct of claim 36 or claim 37, wherein the target gene is exogenous to the eukaryotic cell but is present within the eukaryotic cell.

39. The method, eukaryotic cell or isolated silencing construct of claim 36 or claim 37, wherein the target gene is exogenous to the eukaryotic cell and is not present within the eukaryotic cell.

40. The method, eukaryotic cell or isolated silencing construct of any preceding claim, wherein the first target gene is a homeolog of the second target gene.

41. The method, eukaryotic cell or isolated silencing construct of any preceding claim, wherein the promoter, terminator and/or other regulatory sequence in the silencing insertion is tissue-specific, biotic-condition specific, abiotic-condition specific, inducible, developmentally regulated or constitutive.

42. A plant or a part of plant comprising a eukaryotic cell of any one of claim 3, 4 or 6-41.

43. The method of any of claims 1, 2, 6-41 wherein the eukaryotic cell is a plant cell, and wherein the method further comprises growing a plant from the cell and propagating the plant.

44. The method of claim 43, further comprising harvesting fruit or other plant part or product.

45. A plant produced by the method of claim 43.

46. A seed of the plant of claim 42 or claim 43.

47. An animal or an animal product comprising a eukaryotic cell of any one of claim 3, 4 or 6-41.

48. The method of any of claims 1, 2, 6-41, wherein the eukaryotic cell is an animal cell, and wherein the method further comprises developing an animal from the cell and breeding the animal.

49. The method of claim 48, further comprising producing an animal product.

50. An animal produced by the method of claim 48.

51. A method for treating or preventing disease in a human patient or a livestock animal comprising administering a human or livestock animal cell of any of claims 3, 4, 6-41 or a silencing insertion of any of claims 5-41.


SEQUENCE LISTING (89 sequences)

SEQ ID NO: 1 - DNA, 102 bp, Arabidopsis thaliana
taagtactttcgcttgcagagagaaatcacagtggtcaaaaaagttgtagttttcttaaagtctctttcctctgtgattctctgtgtaagcgaaagagcttg

SEQ ID NO: 2 - DNA, 102 bp, Artificial Sequence
  synthetic polynucleotide
taagtactttgacaatccagccaatccagcagtggtcaaaaaagttgtagttttcttaaagtctctttcctctgctgattggcaggattgtcaaagagcttg

SEQ ID NO: 3 - DNA, 21 bp, Artificial Sequence
  synthetic polynucleotide
tgacaatccagccaatccagc

SEQ ID NO: 4 - DNA, 1701 bp, Arabidopsis thaliana
atggttgtgtttgggaatgtttctgcggcgaatttgccttatcaaaacgggtttttggaggcactttcatctggaggttgtgaactaatgggacatagctttagggttcccacttctcaagcgcttaagacaagaacaaggaggaggagtactgctggtcctttgcaggtagtttgtgtggatattccaaggccagagctagagaacactgtcaatttcttggaagctgctagtttatctgcatccttccgtagtgctcctcgtcctgctaagcctttgaaagttgtaattgctggtgctggattggctggattgtcaactgcaaagtacctggctgatgcaggccacaaacctctgttgcttgaagcaagagatgttcttggtggaaagatagctgcatggaaggatgaagatggggactggtatgagactggtttacatattttcttcggtgcttatccgaatgtgcagaatttatttggagaacttgggatcaatgatcggttgcagtggaaggaacactccatgatttttgctatgccaagtaaacctggagaatttagtagatttgacttcccagatgtcctaccagcacccttaaatggtatttgggctattttgcggaacaacgagatgctgacatggccagagaaaataaagtttgctattggacttttgccagccatggtcggcggtcaggcttatgttgaggcccaagatggtttatcagtcaaagaatggatggaaaagcagggagtacctgagcgcgtgaccgacgaggtgtttattgccatgtcaaaggcgctaaactttataaaccctgatgaactgtcaatgcaatgcattttgatagctttgaaccggtttcttcaggaaaaacatggttccaagatggcattcttggatggtaatcctccggaaaggctttgtatgccagtagtggatcatattcgatcactaggtggggaagtgcaacttaattctaggataaagaaaattgagctcaatgacgatggcacggttaagagtttcttactcactaatggaagcactgtcgaaggagacgcttatgtgtttgccgctccagtcgatatcctgaagctccttttaccagatccctggaaagaaataccgtacttcaagaaattggataaattagttggagtaccagttattaatgttcatatatggtttgatcgaaaactgaagaacacatatgatcacctactctttagcagaagtaaccttctgagcgtgtatgccgacatgtccttaacttgtaaggaatattacgatcctaaccggtcaatgctggagctagtatttgcaccagcagaggaatggatatcacggactgattctgacatcatagatgcaacaatgaaagaacttgagaaactcttccctgatgaaatctcagctgaccaaagcaaagctaaaattctgaagtaccatgtcgttaagactccaagatctgtgtacaagaccatcccaaactgtgaaccatgtcgtcctctacaaagatcacctattgaaggattctacttagctggagattacacaaaacagaagtacttagcttccatggaaggcgctgtcctctctggcaaattctgctctcagtctattgttcaggattacgagctactggctgcgtctggaccaagaaagttgtcggaggcaacagtatcatcatcatga

SEQ ID NO: 5 - DNA, 21 bp, Arabidopsis thaliana
gctggattggctggattgtca

SEQ ID NO: 6 - DNA, 947 bp, Arabidopsis thaliana
atcccaccgtttcttaagactctctctctttctgttttctatttctctctctctcaaatgaaagagagagaagagctcccatggatgaaattagcgagaccgaagtttctccaaggtgatatgtctatctgtatatgtgatacgaagagttagggttttgtcatttcgaagtcaatttttgtttgtttgtcaataatgatatctgaatgatgaagaacacgtaactaagatatgttactgaactatataatacatatgtgtgtttttctgtatctatttctatatatatgtagatgtagtgtaagtctgttatatagacattattcatgtgtacatgcattataccaacataaatttgtatcaatactacttttgatttacgatgatggatgttcttagatatcttcatacgtttgtttccacatgtatttacaactacatatatatttggaatcacatatatacttgattattatagttgtaaagagtaacaagttcttttttcaggcattaaggaaaacataacctccgtgatgcatagagattattggatccgctgtgctgagacattgagtttttcttcggcattccagtttcaatgataaagcggtgttatcctatctgagcttttagtcggattttttcttttcaattattgtgttttatctagatgatgcatttcattattctctttttcttgaccttgtaaggccttttcttgaccttgtaagaccccatctctttctaaacgttttattattttctcgttttacagattctattctatctcttctcaatatagaatagatatctatctctacctctaattcgttcgagtcattttctcctaccttgtctatccctcctgagctaatctccacatatatcttttgtttgttattgatgtatggttgacataaattcaataaagaagttgacgtttttct

SEQ ID NO: 7 - DNA, 947 bp, Artificial Sequence
  synthetic polynucleotide
atcccaccgtttcttaagactctctctctttctgttttctatttctctctctctcaaatgaaagagagagaagagctcccatggatgaaattagcgagaccgaagtttctccaaggtgatatgtctatctgtatatgtgatacgaagagttagggttttgtcatttcgaagtcaatttttgtttgtttgtcaataatgatatctgaatgatgaagaacacgtaactaagatatgttactgaactatataatacatatgtgtgtttttctgtatctatttctatatatatgtagatgtagtgtaagtctgttatatagacattattcatgtgtacatgcattataccaacataaatttgtatcaatactacttttgatttacgatgatggatgttcttagatatcttcatacgtttgtttccacatgtatttacaactacatatatatttggaatcacatatatacttgattattatagttgtaaagagtaacaagttcttttttcaggcattaaggaaaacataacctccgtgatgcatagagattattggatccgctgtgctgagacattgagtttttcttcggcattccagtttcaatgataaagcggtgttatcctatctgagcttttagtcggattttttcttttcaattattgtgttttatctagatgatgcatttcattattctctttttcttgaccttgtaaggccttttcttgaccttgtaagaccccatctctttctaaacgttttattattttctcgttttacagattctattctatctcttctcaatatagaatagatatcggcttcttcagcaccttcaccttacgaattttctcctaccttgtctatccctcctgagctaatctccacatatatcttttgtttgttattgatgtatggttgacataaattcaataaagaagttgacgtttttct

SEQ ID NO: 8 - DNA, 789 bp, Globodera rostochiensis
atggcagtcggaaagaataagaaaatgggcaaaaagggagccaagaagaaggctgtcgatccgttcacacgcaaagaatggtacgacatcaaagcgccggcgatgttcacacatcgaaacgtcggcaaaacgttggtcaaccgtactcagggaacgcgcatttcgagcgactttctaaaaggccgcgtttacgaagtgtcactgggtgaccttaacagcactgacgccgactttcgaaagttccgcctgatctgtgaagaggtacagggcaagatttgcctgaccaactttcacggaatgtcgttcactcgggacaaactgtgctctattgtcaagaagtggcacacgctcattgaggcgaatgtggcagtgaagactaccgacggtttcatgctccgactcttttgtatcggctttaccaagcgaaatgccaatcaaattaagaagacgagctatgcaaaagcctctcaggtgcggatgattcgtgccaaaatggtggagatcatgcagaaagaggtctcttccggcgatctgaaggaagtagtcaacaagctgatcccggattcgatcggcaaagacatagagaaggcgtgctccttctactaccctctgcaggacgtttacattcgtaaggtgaaggtgctgaagaagccgaagttcgagctgggcaaactattggagatgcatggggagggtgccggaacggtcgctacgattacgacggccgccggtgaaaaaattgagagccgtccggatgcgtacgaaccgcctgttcaacagagcgtttga

SEQ ID NO: 9 - DNA, 30 bp, Globodera rostochiensis
attcgtaaggtgaaggtgctgaagaagccg

SEQ ID NO: 10 - DNA, 30 bp, Artificial Sequence
  synthetic polynucleotide
cggcttcttcagcaccttcaccttacgaat

SEQ ID NO: 11 - DNA, 38 bp, Artificial Sequence
  synthetic polynucleotide
cagaagccaaatctttggtaaacaaagtaatttttttg

SEQ ID NO: 12 - DNA, 34 bp, Artificial Sequence
  synthetic polynucleotide
aacttcaatattaaggtttcctgactaccctcac

SEQ ID NO: 13 - DNA, 35 bp, Artificial Sequence
  synthetic polynucleotide
atgaaattaagagccttgtcctagttttgtagtag

SEQ ID NO: 14 - DNA, 42 bp, Artificial Sequence
  synthetic polynucleotide
agtagagacatatacttctgtctacaaacaattgtttttatc

SEQ ID NO: 15 - DNA, 40 bp, Artificial Sequence
  synthetic polynucleotide
  misc_feature (1)..(1): n is a, c, g, or t
  misc_feature (2)..(3): 6nt restriction site for high-fidelity enzyme
nncagaagccaaatctttggtaaacaaagtaatttttttg

SEQ ID NO: 16 - DNA, 36 bp, Artificial Sequence
  synthetic polynucleotide
  misc_feature (1)..(1): n is a, c, g, or t
  misc_feature (2)..(3): 6nt restriction site for high-fidelity enzyme
nnaacttcaatattaaggtttcctgactaccctcac

SEQ ID NO: 17 - DNA, 37 bp, Artificial Sequence
  synthetic polynucleotide
  misc_feature (1)..(1): n is a, c, g, or t
  misc_feature (2)..(3): 6nt restriction site for high-fidelity enzyme
nnatgaaattaagagccttgtcctagttttgtagtag

SEQ ID NO: 18 - DNA, 44 bp, Artificial Sequence
  synthetic polynucleotide
  misc_feature (1)..(1): n is a, c, g, or t
  misc_feature (2)..(3): 6nt restriction site for high-fidelity enzyme
nnagtagagacatatacttctgtctacaaacaattgtttttatc

SEQ ID NO: 19 - DNA, 38 bp, Artificial Sequence; identical to SEQ ID NO: 11
  synthetic polynucleotide
  misc_feature (1)..(2): phosphorothioate linkage

SEQ ID NO: 20 - DNA, 34 bp, Artificial Sequence; identical to SEQ ID NO: 12
  synthetic polynucleotide
  misc_feature (1)..(2): phosphorothioate linkage

SEQ ID NO: 21 - DNA, 35 bp, Artificial Sequence; identical to SEQ ID NO: 13
  synthetic polynucleotide
  misc_feature (1)..(2): phosphorothioate linkage

SEQ ID NO: 22 - DNA, 42 bp, Artificial Sequence; identical to SEQ ID NO: 14
  synthetic polynucleotide
  misc_feature (1)..(2): phosphorothioate linkage

SEQ ID NO: 23 - DNA, 40 bp, Artificial Sequence
  synthetic polynucleotide
  misc_feature (1)..(1): n is a, c, g, or t
  misc_feature (2)..(3): 6nt unique restriction site for high-fidelity enzyme
nncatatcaagctagattttgaattaccctatttcatcgt

SEQ ID NO: 24 - DNA, 38 bp, Artificial Sequence
  synthetic polynucleotide
  misc_feature (1)..(2): phosphorothioate linkage
catatcaagctagattttgaattaccctatttcatcgt

SEQ ID NO: 25 - DNA, 22 bp, Artificial Sequence
  synthetic polynucleotide
acaaactacctgcaagtggtca

SEQ ID NO: 26 - DNA, 19 bp, Artificial Sequence
  synthetic polynucleotide
caagctctttatccacact

SEQ ID NO: 27 - DNA, 20 bp, Artificial Sequence
  synthetic polynucleotide
  misc_feature (1)..(2): phosphorothioate linkage
aattcaaagccaatggaact

SEQ ID NO: 28 - DNA, 20 bp, Artificial Sequence
  synthetic polynucleotide
  misc_feature (1)..(2): phosphorothioate linkage
agtacaagatgtcaaatgcc

SEQ ID NO: 29 - DNA, 21 bp, Artificial Sequence
  synthetic polynucleotide
ggtaactcatttgggatggta

SEQ ID NO: 30 - DNA, 19 bp, Artificial Sequence
  synthetic polynucleotide
gaacggtcgctacgattac

SEQ ID NO: 31 - DNA, 19 bp, Artificial Sequence
  synthetic polynucleotide
aatccagcagtggtcaaaa

SEQ ID NO: 32 - DNA, 19 bp, Artificial Sequence
  synthetic polynucleotide
caagctctttgacaatcct

SEQ ID NO: 33 - DNA, 20 bp, Artificial Sequence
  synthetic polynucleotide
gagcttcctcaaagggctga

SEQ ID NO: 34 - DNA, 25 bp, Artificial Sequence
  synthetic polynucleotide
tggttacgaatgaatttaatgggga

SEQ ID NO: 35 - DNA, 16 bp, Artificial Sequence
  synthetic polynucleotide
acgatggactccagag

SEQ ID NO: 36 - DNA, 33 bp, Artificial Sequence
  synthetic polynucleotide
  misc_feature (25)..(25): n is a, c, g, or t
  misc_feature (27)..(29): n is a, c, g, or t
acgatggactccagagcggccgcvnvnnnggaa

SEQ ID NO: 37 - DNA, 33 bp, Artificial Sequence
  synthetic polynucleotide
  misc_feature (25)..(25): n is a, c, g, or t
  misc_feature (27)..(29): n is a, c, g, or t
acgatggactccagagcggccgcbnbnnnggtt

SEQ ID NO: 38 - DNA, 34 bp, Artificial Sequence
  synthetic polynucleotide
  misc_feature (26)..(26): n is a, c, g, or t
  misc_feature (28)..(30): n is a, c, g, or t
acgatggactccagagcggccgcvvnvnnnccaa

SEQ ID NO: 39 - DNA, 34 bp, Artificial Sequence
  synthetic polynucleotide
  misc_feature (26)..(26): n is a, c, g, or t
  misc_feature (28)..(30): n is a, c, g, or t
acgatggactccagagcggccgcbdnbnnncggt

SEQ ID NO: 40 - DNA, 36 bp, Artificial Sequence
  synthetic polynucleotide
taagtactttgacaatccagccaatccagcagtggt

SEQ ID NO: 41 - DNA, 34 bp, Artificial Sequence
  synthetic polynucleotide
gacaatccagccaatccagcagtggtcaaaaaag

SEQ ID NO: 42 - DNA, 36 bp, Artificial Sequence
  synthetic polynucleotide
ctctgctgattggcaggattgtcaaagagcttgctc

SEQ ID NO: 43 - RNA, 43 nt, Artificial Sequence
  synthetic polynucleotide
uauuauuucaaaguaguugcuggaggcuaguuuacauaguuga

SEQ ID NO: 44 - RNA, 43 nt, Artificial Sequence
  synthetic polynucleotide
uuucgugggguucacuaaucugguuuagucggcggaugagugu

SEQ ID NO: 45 - RNA, 43 nt, Artificial Sequence
  synthetic polynucleotide
uuuauuguucauguaguaggcuucuacaauauuagcgcugaag

SEQ ID NO: 46 - RNA, 43 nt, Artificial Sequence
  synthetic polynucleotide
uuucgugggguucacuaaucucgguuaauggggcccaaggaag

SEQ ID NO: 47 - RNA, 43 nt, Artificial Sequence
  synthetic polynucleotide
uuacauguugaugaacuuccugagggguucagcgguauguaaa

SEQ ID NO: 48 - RNA, 43 nt, Artificial Sequence
  synthetic polynucleotide
uuuauuguucauguaguaggcugccacuacagggauagugaau

SEQ ID NO: 49 - RNA, 43 nt, Artificial Sequence
  synthetic polynucleotide
uuucgugggguucacuaaucucagauagugguuuccacggaaa

SEQ ID NO: 50 - DNA, 21 bp, Artificial Sequence
  synthetic polynucleotide
tcacatgtgcatcctctctga

SEQ ID NO: 51 - DNA, 22 bp, Artificial Sequence
  synthetic polynucleotide
ggctagtccgttatcaacttga

SEQ ID NO: 52 - DNA, 19 bp, Artificial Sequence
  synthetic polynucleotide
gaagcctactacatgaaca

SEQ ID NO: 53 - DNA, 22 bp, Artificial Sequence
  synthetic polynucleotide
tagtccactggttaatcagaga

SEQ ID NO: 54 - DNA, 26 bp, Artificial Sequence
  synthetic polynucleotide
gttcagagttctacagtccgacgatc

SEQ ID NO: 55 - DNA, 21 bp, Artificial Sequence
  synthetic polynucleotide
agatcggaagagcacacgtct

SEQ ID NO: 56 - DNA, 40 bp, Artificial Sequence
  synthetic polynucleotide
atgtttgaacgatcgggcccaagggacacgaagtgatccg

SEQ ID NO: 57 - DNA, 38 bp, Artificial Sequence
  synthetic polynucleotide
ctccaccatgttcccgggggcacagagtgttcaacccc

SEQ ID NO: 58 - DNA, 19 bp, Artificial Sequence
  synthetic polynucleotide
gattagtgaaccccacgaa

SEQ ID NO: 59 - DNA, 1002 bp, Arabidopsis thaliana
cagaagccaaatctttggtaaacaaagtaatttttttgccatttgattggttagtataggagaatttaaaaacgacgataaggtttaggtaaattatttcatttgaaaataattgagcaccgttaataattttcatccataaaataatatttcaaagatgatatttgatccccattaaattcattcgtaaccaaaaaaaagttatgaaaaaagagtggtcgtgtgagttgcccaagcaccattataataaaaaataaaataattagcaagtaataaggaataaaatcctgtaattatagctgaaaaaggaaaaatatttggagaccgtcagattcgaatctgaacaaagcataaaaaagtcaacaaaacttaaagcggcggtctcatcgtaatctcagcccaataccctattttcctctcccctatataaatactttcttcttctactgatcttcttctcacaaataaacccaaatatatcaatctactgtgttggtgattaagtactttcgcttgcagagagaaatcacagtggtcaaaaaagttgtagttttcttaaagtctctttcctctgtgattctctgtgtaagcgaaagagcttgctccctaaacttatctctctgatgatttaatgttagagatcttcgtaaatctatgtgtttgatagatctgatgcgttttttgagttgatgatttgattatttttcactggaaagtatctcattagggtaacgataatgttttatggatttggttgtataacagatccatgaaatcttgactggttataaaatctgaataatgtatttcaatttggagattcggtgataaaaattactgatttacgaatgacatttatatcgatagatgagtttgctgatttggttgttaaattgataaatcaaggacatgagaaactgtttttgtatgctaatttgtccatggaataaaattgggaggtgaggaccgtgagggtagtcaggaaaccttaatattgaagtt

SEQ ID NO: 60 - DNA, 500 bp, Arabidopsis thaliana
cagaagccaaatctttggtaaacaaagtaatttttttgccatttgattggttagtataggagaatttaaaaacgacgataaggtttaggtaaattatttcatttgaaaataattgagcaccgttaataattttcatccataaaataatatttcaaagatgatatttgatccccattaaattcattcgtaaccaaaaaaaagttatgaaaaaagagtggtcgtgtgagttgcccaagcaccattataataaaaaataaaataattagcaagtaataaggaataaaatcctgtaattatagctgaaaaaggaaaaatatttggagaccgtcagattcgaatctgaacaaagcataaaaaagtcaacaaaacttaaagcggcggtctcatcgtaatctcagcccaataccctattttcctctcccctatataaatactttcttcttctactgatcttcttctcacaaataaacccaaatatatcaatctactgtgttggtgat

SEQ ID NO: 61 - DNA, 400 bp, Arabidopsis thaliana
ctccctaaacttatctctctgatgatttaatgttagagatcttcgtaaatctatgtgtttgatagatctgatgcgttttttgagttgatgatttgattatttttcactggaaagtatctcattagggtaacgataatgttttatggatttggttgtataacagatccatgaaatcttgactggttataaaatctgaataatgtatttcaatttggagattcggtgataaaaattactgatttacgaatgacatttatatcgatagatgagtttgctgatttggttgttaaattgataaatcaaggacatgagaaactgtttttgtatgctaatttgtccatggaataaaattgggaggtgaggaccgtgagggtagtcaggaaaccttaatattgaagtt

SEQ ID NO: 62 - DNA, 1002 bp, Artificial Sequence
  synthetic polynucleotide
cagaagccaaatctttggtaaacaaagtaatttttttgccatttgattggttagtataggagaatttaaaaacgacgataaggtttaggtaaattatttcatttgaaaataattgagcaccgttaataattttcatccataaaataatatttcaaagatgatatttgatccccattaaattcattcgtaaccaaaaaaaagttatgaaaaaagagtggtcgtgtgagttgcccaagcaccattataataaaaaataaaataattagcaagtaataaggaataaaatcctgtaattatagctgaaaaaggaaaaatatttggagaccgtcagattcgaatctgaacaaagcataaaaaagtcaacaaaacttaaagcggcggtctcatcgtaatctcagcccaataccctattttcctctcccctatataaatactttcttcttctactgatcttcttctcacaaataaacccaaatatatcaatctactgtgttggtgattaagtactttgacaatccagccaatccagcagtggtcaaaaaagttgtagttttcttaaagtctctttcctctgctgattggcaggattgtcaaagagcttgctccctaaacttatctctctgatgatttaatgttagagatcttcgtaaatctatgtgtttgatagatctgatgcgttttttgagttgatgatttgattatttttcactggaaagtatctcattagggtaacgataatgttttatggatttggttgtataacagatccatgaaatcttgactggttataaaatctgaataatgtatttcaatttggagattcggtgataaaaattactgatttacgaatgacatttatatcgatagatgagtttgctgatttggttgttaaattgataaatcaaggacatgagaaactgtttttgtatgctaatttgtccatggaataaaattgggaggtgaggaccgtgagggtagtcaggaaaccttaatattgaagtt

SEQ ID NO: 63 - DNA, 2647 bp, Arabidopsis thaliana
atgaaattaagagccttgtcctagttttgtagtagtattttgttataaaaaaatattgcaattgaaaaataaaggttttactgaataccaccaatgtgtgtgttaaactaataaacatatttgtacattacgcgaaacattaattaatttaggaacgaaggtcaagggattcttttttggcattatcaaatgatataacaatgttaatagttactataagttgtgatattactatttttttacattattttgaccattttatgtcttagaagactgtgtgattcccccgcacgcccacaaagtttctattccccacaaatctcagacttttgattactggattttagaaataagaataataacttgctcgaaagagattcgaccttctttatcttacttcattttaaagaaaactgataagccattgaataagaacataattcctctttttttttgttagaatgagaagagagcaaagaaacttcttaaaatgttataaaactaattaatactaggaagactataagcttgaaatatataaactattacagagtaagtactacatatataaaaaacacagtgtgtgtaaacatgatttattttgaaatttgaactttgatgtggttttttttgccctagaattgaggaattccatttaccaagattctcttaatttgctcaaactcagtctcttcaccaattagtggataattatgtttggctaggatgcctagtccagctagattagattatgaagtatataccaaacaatgttcatttatcaaattttcaattaacaatcaacgaaattttaaaatagaagctctacgaaaaatacatttaacatgagaaaccaacatatattttaaaaactgttgataaacaataagaaaaaaacttgagacacagtttcgagttttttatttgacctgttgactttggtttcgcttaaggaccatagaaactgctaactcaggaaaaggaccaccttcgggcacaacggtcaggtgttcgggcagattggtctatgccatatcgcaacgatgacgtcaccaaattcatattttaaaactcgtttcgggcaacgacaacgtcatgatccctcccaaaggtctaattgggccccggcccacaaaggttatcatcttctttcttcttctttgtttattgttgcttttgccttaaaatctcttcttcatcatcccaccgtttcttaagactctctctctttctgttttctatttctctctctctcaaatgaaagagagagaagagctcccatggatgaaattagcgagaccgaagtttctccaaggtgatatgtctatctgtatatgtgatacgaagagttagggttttgtcatttcgaagtcaatttttgtttgtttgtcaataatgatatctgaatgatgaagaacacgtaactaagatatgttactgaactatataatacatatgtgtgtttttctgtatctatttctatatatatgtagatgtagtgtaagtctgttatatagacattattcatgtgtacatgcattataccaacataaatttgtatcaatactacttttgatttacgatgatggatgttcttagatatcttcatacgtttgtttccacatgtatttacaactacatatatatttggaatcacatatatacttgattattatagttgtaaagagtaacaagttcttttttcaggcattaaggaaaacataacctccgtgatgcatagagattattggatccgctgtgctgagacattgagtttttcttcggcattccagtttcaatgataaagcggtgttatcctatctgagcttttagtcggattttttcttttcaattattgtgttttatctagatgatgcatttcattattctctttttcttgaccttgtaaggccttttcttgaccttgtaagaccccatctctttctaaacgttttattattttctcgttttacagattctattctatctcttctcaatatagaatagatatctatctctacctctaattcgttcgagtcattttctcctaccttgtctatccctcctgagctaatctccacatatatcttttgtttgttattgatgtatggttgacataaattcaataaagaagttgacgtttttcttatttgatttttgttgttgttggttatattattgcaacaaaattaaagggggtaaggaaggtctcgctatcaaggggactggcaaaaggtaatgaataaggaaacgggcaaaaagaattatgcctttactctctcttttaaggctttggacaggaatttagttttgttttatgtgttgtgttgtttgtttgggtctgactgaccccaaagggcaaagccaaaccagagaagactcttattaaatattccctcagaatcatttattgcctctatctttatctctctctttctcacactcgtgactgtttctcaccttatgtatgtactagtaatagtttttaccactttcaacttttacaaatagcatttgtttctgtttaaaacggttgacggataggtaatacccaaagttttatacaccaagaggcgaggagttttaagtttataggtcgttcatagataaaaacaattgtttgtagacagaagtatatgtctctact

SEQ ID NO: 64 - DNA, 1200 bp, Arabidopsis thaliana
atgaaattaagagccttgtcctagttttgtagtagtattttgttataaaaaaatattgcaattgaaaaataaaggttttactgaataccaccaatgtgtgtgttaaactaataaacatatttgtacattacgcgaaacattaattaatttaggaacgaaggtcaagggattcttttttggcattatcaaatgatataacaatgttaatagttactataagttgtgatattactatttttttacattattttgaccattttatgtcttagaagactgtgtgattcccccgcacgcccacaaagtttctattccccacaaatctcagacttttgattactggattttagaaataagaataataacttgctcgaaagagattcgaccttctttatcttacttcattttaaagaaaactgataagccattgaataagaacataattcctctttttttttgttagaatgagaagagagcaaagaaacttcttaaaatgttataaaactaattaatactaggaagactataagcttgaaatatataaactattacagagtaagtactacatatataaaaaacacagtgtgtgtaaacatgatttattttgaaatttgaactttgatgtggttttttttgccctagaattgaggaattccatttaccaagattctcttaatttgctcaaactcagtctcttcaccaattagtggataattatgtttggctaggatgcctagtccagctagattagattatgaagtatataccaaacaatgttcatttatcaaattttcaattaacaatcaacgaaattttaaaatagaagctctacgaaaaatacatttaacatgagaaaccaacatatattttaaaaactgttgataaacaataagaaaaaaacttgagacacagtttcgagttttttatttgacctgttgactttggtttcgcttaaggaccatagaaactgctaactcaggaaaaggaccaccttcgggcacaacggtcaggtgttcgggcagattggtctatgccatatcgcaacgatgacgtcaccaaattcatattttaaaactcgtttcgggcaacgacaacgtcatgatccctcccaaaggtctaattgggccccggcccacaaaggttatcatcttctttcttcttctttgtttattgttgcttttgccttaaaatctcttcttcatc

SEQ ID NO: 65 - DNA, 500 bp, Arabidopsis thaliana
tatttgatttttgttgttgttggttatattattgcaacaaaattaaagggggtaaggaaggtctcgctatcaaggggactggcaaaaggtaatgaataaggaaacgggcaaaaagaattatgcctttactctctcttttaaggctttggacaggaatttagttttgttttatgtgttgtgttgtttgtttgggtctgactgaccccaaagggcaaagccaaaccagagaagactcttattaaatattccctcagaatcatttattgcctctatctttatctctctctttctcacactcgtgactgtttctcaccttatgtatgtactagtaatagtttttaccactttcaacttttacaaatagcatttgtttctgtttaaaacggttgacggataggtaatacccaaagttttatacaccaagaggcgaggagttttaagtttataggtcgttcatagataaaaacaattgtttgtagacagaagtatatgtctctact

SEQ ID NO: 66 - DNA, 2647 bp, Artificial Sequence
  synthetic polynucleotide
atgaaattaagagccttgtcctagttttgtagtagtattttgttataaaaaaatattgcaattgaaaaataaaggttttactgaataccaccaatgtgtgtgttaaactaataaacatatttgtacattacgcgaaacattaattaatttaggaacgaaggtcaagggattcttttttggcattatcaaatgatataacaatgttaatagttactataagttgtgatattactatttttttacattattttgaccattttatgtcttagaagactgtgtgattcccccgcacgcccacaaagtttctattccccacaaatctcagacttttgattactggattttagaaataagaataataacttgctcgaaagagattcgaccttctttatcttacttcattttaaagaaaactgataagccattgaataagaacataattcctctttttttttgttagaatgagaagagagcaaagaaacttcttaaaatgttataaaactaattaatactaggaagactataagcttgaaatatataaactattacagagtaagtactacatatataaaaaacacagtgtgtgtaaacatgatttattttgaaatttgaactttgatgtggttttttttgccctagaattgaggaattccatttaccaagattctcttaatttgctcaaactcagtctcttcaccaattagtggataattatgtttggctaggatgcctagtccagctagattagattatgaagtatataccaaacaatgttcatttatcaaattttcaattaacaatcaacgaaattttaaaatagaagctctacgaaaaatacatttaacatgagaaaccaacatatattttaaaaactgttgataaacaataagaaaaaaacttgagacacagtttcgagttttttatttgacctgttgactttggtttcgcttaaggaccatagaaactgctaactcaggaaaaggaccaccttcgggcacaacggtcaggtgttcgggcagattggtctatgccatatcgcaacgatgacgtcaccaaattcatattttaaaactcgtttcgggcaacgacaacgtcatgatccctcccaaaggtctaattgggccccggcccacaaaggttatcatcttctttcttcttctttgtttattgttgcttttgccttaaaatctcttcttcatcatcccaccgtttcttaagactctctctctttctgttttctatttctctctctctcaaatgaaagagagagaagagctcccatggatgaaattagcgagaccgaagtttctccaaggtgatatgtctatctgtatatgtgatacgaagagttagggttttgtcatttcgaagtcaatttttgtttgtttgtcaataatgatatctgaatgatgaagaacacgtaactaagatatgttactgaactatataatacatatgtgtgtttttctgtatctatttctatatatatgtagatgtagtgtaagtctgttatatagacattattcatgtgtacatgcattataccaacataaatttgtatcaatactacttttgatttacgatgatggatgttcttagatatcttcatacgtttgtttccacatgtatttacaactacatatatatttggaatcacatatatacttgattattatagttgtaaagagtaacaagttcttttttcaggcattaaggaaaacataacctccgtgatgcatagagattattggatccgctgtgctgagacattgagtttttcttcggcattccagtttcaatgataaagcggtgttatcctatctgagcttttagtcggattttttcttttcaattattgtgttttatctagatgatgcatttcattattctctttttcttgaccttgtaaggccttttcttgaccttgtaagaccccatctctttctaaacgttttattattttctcgttttacagattctattctatctcttctcaatatagaatagatatcggcttcttcagcaccttcaccttacgaattttctcctaccttgtctatccctcctgagctaatctccacatatatcttttgtttgttattgatgtatggttgacataaattcaataaagaagttgacgtttttcttatttgatttttgttgttgttggttatattattgcaacaaaattaaagggggtaaggaaggtctcgctatcaaggggactggcaaaaggtaatgaataaggaaacgggcaaaaagaattatgcctttactctctcttttaaggctttggacaggaatttagttttgttttatgtgttgtgttgtttgtttgggtctgactgaccccaaagggcaaagccaaaccagagaagactcttattaaatattccctcagaatcatttattgcctctatctttatctctctctttctcacactcgtgactgtttctcaccttatgtatgtactagtaatagtttttaccactttcaacttttacaaatagcatttgtttctgtttaaaacggttgacggataggtaatacccaaagttttatacaccaagaggcgaggagttttaagtttataggtcgttcatagataaaaacaattgtttgtagacagaagtatatgtctctact

SEQ ID NO: 67 - DNA, 2013 bp, Arabidopsis thaliana
atggcggcggcaacaacaacaacaacaacatcttcttcgatctccttctccaccaaaccatctccttcctcctccaaatcaccattaccaatctccagattctccctcccattctccctaaaccccaacaaatcatcctcctcctcccgccgccgcggtatcaaatccagctctccctcctccatctccgccgtgctcaacacaaccaccaatgtcacaaccactccctctccaaccaaacctaccaaacccgaaacattcatctcccgattcgctccagatcaaccccgcaaaggcgctgatatcctcgtcgaagctttagaacgtcaaggcgtagaaaccgtattcgcttaccctggaggtgcatcaatggagattcaccaagccttaacccgctcttcctcaatccgtaacgtccttcctcgtcacgaacaaggaggtgtattcgcagcagaaggatacgctcgatcctcaggtaaaccaggtatctgtatagccacttcaggtcccggagctacaaatctcgttagcggattagccgatgcgttgttagatagtgttcctcttgtagcaatcacaggacaagtccctcgtcgtatgattggtacagatgcgtttcaagagactccgattgttgaggtaacgcgttcgattacgaagcataactatcttgtgatggatgttgaagatatccctaggattattgaggaagctttctttttagctacttctggtagacctggacctgttttggttgatgttcctaaagatattcaacaacagcttgcgattcctaattgggaacaggctatgagattacctggttatatgtctaggatgcctaaacctccggaagattctcatttggagcagattgttaggttgatttctgagtctaagaagcctgtgttgtatgttggtggtggttgtttgaattctagcgatgaattgggtaggtttgttgagcttacggggatccctgttgcgagtacgttgatggggctgggatcttatccttgtgatgatgagttgtcgttacatatgcttggaatgcatgggactgtgtatgcaaattacgctgtggagcatagtgatttgttgttggcgtttggggtaaggtttgatgatcgtgtcacgggtaagcttgaggcttttgctagtagggctaagattgttcatattgatattgactcggctgagattgggaagaataagactcctcatgtgtctgtgtgtggtgatgttaagctggctttgcaagggatgaataaggttcttgagaaccgagcggaggagcttaagcttgattttggagtttggaggaatgagttgaacgtacagaaacagaagtttccgttgagctttaagacgtttggggaagctattcctccacagtatgcgattaaggtccttgatgagttgactgatggaaaagccataataagtactggtgtcgggcaacatcaaatgtgggcggcgcagttctacaattacaagaaaccaaggcagtggctatcatcaggaggccttggagctatgggatttggacttcctgctgcgattggagcgtctgttgctaaccctgatgcgatagttgtggatattgacggagatggaagctttataatgaatgtgcaagagctagccactattcgtgtagagaatcttccagtgaaggtacttttattaaacaaccagcatcttggcatggttatgcaatgggaagatcggttctacaaagctaaccgagctcacacatttctcggggatccggctcaggaggacgagatattcccgaacatgttgctgtttgcagcagcttgcgggattccagcggcgagggtgacaaagaaagcagatctccgagaagctattcagacaatgctggatacaccaggaccttacctgttggatgtgatttgtccgcaccaagaacatgtgttgccgatgatcccgagtggtggcactttcaacgatgtcataacggaaggagatggccggattaaatactga

SEQ ID NO: 68 - DNA, 2013 bp, Artificial Sequence
  synthetic polynucleotide
atggcggcggcaacaacaacaacaacaacatcttcttcgatctccttctccaccaaaccatctccttcctcctccaaatcaccattaccaatctccagattctccctcccattctccctaaaccccaacaaatcatcctcctcctcccgccgccgcggtatcaaatccagctctccctcctccatctccgccgtgctcaacacaaccaccaatgtcacaaccactccctctccaaccaaacctaccaaacccgaaacattcatctcccgattcgctccagatcaaccccgcaaaggcgctgatatcctcgtcgaagctttagaacgtcaaggcgtagaaaccgtattcgcttaccctggaggtgcatcaatggagattcaccaagccttaacccgctcttcctcaatccgtaacgtccttcctcgtcacgaacaaggaggtgtattcgcagcagaaggatacgctcgatcctcaggtaaaccaggtatctgtatagccacttcaggtcccggagctacaaatctcgttagcggattagccgatgcgttgttagatagtgttcctcttgtagcaatcacaggacaagtctctcgtcgtatgattggtacagatgcgtttcaagagactccgattgttgaggtaacgcgttcgattacgaagcataactatcttgtgatggatgttgaagatatccctaggattattgaggaagctttctttttagctacttctggtagacctggacctgttttggttgatgttcctaaagatattcaacaacagcttgcgattcctaattgggaacaggctatgagattacctggttatatgtctaggatgcctaaacctccggaagattctcatttggagcagattgttaggttgatttctgagtctaagaagcctgtgttgtatgttggtggtggttgtttgaattctagcgatgaattgggtaggtttgttgagcttacggggatccctgttgcgagtacgttgatggggctgggatcttatccttgtgatgatgagttgtcgttacatatgcttggaatgcatgggactgtgtatgcaaattacgctgtggagcatagtgatttgttgttggcgtttggggtaaggtttgatgatcgtgtcacgggtaagcttgaggcttttgctagtagggctaagattgttcatattgatattgactcggctgagattgggaagaataagactcctcatgtgtctgtgtgtggtgatgttaagctggctttgcaagggatgaataaggttcttgagaaccgagcggaggagcttaagcttgattttggagtttggaggaatgagttgaacgtacagaaacagaagtttccgttgagctttaagacgtttggggaagctattcctccacagtatgcgattaaggtccttgatgagttgactgatggaaaagccataataagtactggtgtcgggcaacatcaaatgtgggcggcgcagttctacaattacaagaaaccaaggcagtggctatcatcaggaggccttggagctatgggatttggacttcctgctgcgattggagcgtctgttgctaaccctgatgcgatagttgtggatattgacggagatggaagctttataatgaatgtgcaagagctagccactattcgtgtagagaatcttccagtgaaggtacttttattaaacaaccagcatcttggcatggttatgcaatgggaagatcggttctacaaagctaaccgagctcacacatttctcggggatccggctcaggaggacgagatattcccgaacatgttgctgtttgcagcagcttgcgggattccagcggcgagggtgacaaagaaagcagatctccgagaagctattcagacaatgctggatacaccaggaccttacctgttggatgtgatttgtccgcaccaagaacatgtgttgccgatgatcccgagtggtggcactttcaacgatgtcataacggaaggagatggccggattaaatactga

SEQ ID NO: 69 - DNA, 291 bp, Arabidopsis thaliana
atggcggcggcaacaacaacaacaacaacatcttcttcgatctccttctccaccaaaccatctccttcctcctccaaatcaccattaccaatctccagattctccctcccattctccctaaaccccaacaaatcatcctcctcctcccgccgccgcggtatcaaatccagctctccctcctccatctccgccgtgctcaacacaaccaccaatgtcacaaccactccctctccaaccaaacctaccaaacccgaaacattcatctcccgattcgctccagatcaaccccgc

SEQ ID NO: 70 - DNA, 1719 bp, Arabidopsis thaliana
aaaggcgctgatatcctcgtcgaagctttagaacgtcaaggcgtagaaaccgtattcgcttaccctggaggtgcatcaatggagattcaccaagccttaacccgctcttcctcaatccgtaacgtccttcctcgtcacgaacaaggaggtgtattcgcagcagaaggatacgctcgatcctcaggtaaaccaggtatctgtatagccacttcaggtcccggagctacaaatctcgttagcggattagccgatgcgttgttagatagtgttcctcttgtagcaatcacaggacaagtccctcgtcgtatgattggtacagatgcgtttcaagagactccgattgttgaggtaacgcgttcgattacgaagcataactatcttgtgatggatgttgaagatatccctaggattattgaggaagctttctttttagctacttctggtagacctggacctgttttggttgatgttcctaaagatattcaacaacagcttgcgattcctaattgggaacaggctatgagattacctggttatatgtctaggatgcctaaacctccggaagattctcatttggagcagattgttaggttgatttctgagtctaagaagcctgtgttgtatgttggtggtggttgtttgaattctagcgatgaattgggtaggtttgttgagcttacggggatccctgttgcgagtacgttgatggggctgggatcttatccttgtgatgatgagttgtcgttacatatgcttggaatgcatgggactgtgtatgcaaattacgctgtggagcatagtgatttgttgttggcgtttggggtaaggtttgatgatcgtgtcacgggtaagcttgaggcttttgctagtagggctaagattgttcatattgatattgactcggctgagattgggaagaataagactcctcatgtgtctgtgtgtggtgatgttaagctggctttgcaagggatgaataaggttcttgagaaccgagcggaggagcttaagcttgattttggagtttggaggaatgagttgaacgtacagaaacagaagtttccgttgagctttaagacgtttggggaagctattcctccacagtatgcgattaaggtccttgatgagttgactgatggaaaagccataataagtactggtgtcgggcaacatcaaatgtgggcggcgcagttctacaattacaagaaaccaaggcagtggctatcatcaggaggccttggagctatgggatttggacttcctgctgcgattggagcgtctgttgctaaccctgatgcgatagttgtggatattgacggagatggaagctttataatgaatgtgcaagagctagccactattcgtgtagagaatcttccagtgaaggtacttttattaaacaaccagcatcttggcatggttatgcaatgggaagatcggttctacaaagctaaccgagctcacacatttctcggggatccggctcaggaggacgagatattcccgaacatgttgctgtttgcagcagcttgcgggattccagcggcgagggtgacaaagaaagcagatctccgagaagctattcagacaatgctggatacaccaggaccttacctgttggatgtgatttgtccgcaccaagaacatgtgttgccgatgatcccgagtggtggcactttcaacgatgtcataacggaaggagatggccggattaaatac

SEQ ID NO: 71 - DNA, 1719 bp, Artificial Sequence
  synthetic polynucleotide
aaaggcgctgatatcctcgtcgaagctttagaacgtcaaggcgtagaaaccgtattcgcttaccctggaggtgcatcaatggagattcaccaagccttaacccgctcttcctcaatccgtaacgtccttcctcgtcacgaacaaggaggtgtattcgcagcagaaggatacgctcgatcctcaggtaaaccaggtatctgtatagccacttcaggtcccggagctacaaatctcgttagcggattagccgatgcgttgttagatagtgttcctcttgtagcaatcacaggacaagtctctcgtcgtatgattggtacagatgcgtttcaagagactccgattgttgaggtaacgcgttcgattacgaagcataactatcttgtgatggatgttgaagatatccctaggattattgaggaagctttctttttagctacttctggtagacctggacctgttttggttgatgttcctaaagatattcaacaacagcttgcgattcctaattgggaacaggctatgagattacctggttatatgtctaggatgcctaaacctccggaagattctcatttggagcagattgttaggttgatttctgagtctaagaagcctgtgttgtatgttggtggtggttgtttgaattctagcgatgaattgggtaggtttgttgagcttacggggatccctgttgcgagtacgttgatggggctgggatcttatccttgtgatgatgagttgtcgttacatatgcttggaatgcatgggactgtgtatgcaaattacgctgtggagcatagtgatttgttgttggcgtttggggtaaggtttgatgatcgtgtcacgggtaagcttgaggcttttgctagtagggctaagattgttcatattgatattgactcggctgagattgggaagaataagactcctcatgtgtctgtgtgtggtgatgttaagctggctttgcaagggatgaataaggttcttgagaaccgagcggaggagcttaagcttgattttggagtttggaggaatgagttgaacgtacagaaacagaagtttccgttgagctttaagacgtttggggaagctattcctccacagtatgcgattaaggtccttgatgagttgactgatggaaaagccataataagtactggtgtcgggcaacatcaaatgtgggcggcgcagttctacaattacaagaaaccaaggcagtggctatcatcaggaggccttggagctatgggatttggacttcctgctgcgattggagcgtctgttgctaaccctgatgcgatagttgtggatattgacggagatggaagctttataatgaatgtgcaagagctagccactattcgtgtagagaatcttccagtgaaggtacttttattaaacaaccagcatcttggcatggttatgcaatgggaagatcggttctacaaagctaaccgagctcacacatttctcggggatccggctcaggaggacgagatattcccgaacatgttgctgtttgcagcagcttgcgggattccagcggcgagggtgacaaagaaagcagatctccgagaagctattcagacaatgctggatacaccaggaccttacctgttggatgtgatttgtccgcaccaagaacatgtgttgccgatgatcccgagtggtggcactttcaacgatgtcataacggaaggagatggccggattaaatac

SEQ ID NO: 72 - protein, 670 aa, Arabidopsis thaliana
MAAATTTTTTSSSISFSTKPSPSSSKSPLPISRFSLPFSLNPNKSSSSSRRRGIKSSSPSSISAVLNTTTNVTTTPSPTKPTKPETFISRFAPDQPRKGADILVEALERQGVETVFAYPGGASMEIHQALTRSSSIRNVLPRHEQGGVFAAEGYARSSGKPGICIATSGPGATNLVSGLADALLDSVPLVAITGQVPRRMIGTDAFQETPIVEVTRSITKHNYLVMDVEDIPRIIEEAFFLATSGRPGPVLVDVPKDIQQQLAIPNWEQAMRLPGYMSRMPKPPEDSHLEQIVRLISESKKPVLYVGGGCLNSSDELGRFVELTGIPVASTLMGLGSYPCDDELSLHMLGMHGTVYANYAVEHSDLLLAFGVRFDDRVTGKLEAFASRAKIVHIDIDSAEIGKNKTPHVSVCGDVKLALQGMNKVLENRAEELKLDFGVWRNELNVQKQKFPLSFKTFGEAIPPQYAIKVLDELTDGKAIISTGVGQHQMWAAQFYNYKKPRQWLSSGGLGAMGFGLPAAIGASVANPDAIVVDIDGDGSFIMNVQELATIRVENLPVKVLLLNNQHLGMVMQWEDRFYKANRAHTFLGDPAQEDEIFPNMLLFAAACGIPAARVTKKADLREAIQTMLDTPGPYLLDVICPHQEHVLPMIPSGGTFNDVITEGDGRIKY

SEQ ID NO: 73 - protein, 670 aa, Artificial Sequence; identical to SEQ ID NO: 72
  synthetic polypeptide

SEQ ID NO: 74 - protein, 97 aa, Arabidopsis thaliana
MAAATTTTTTSSSISFSTKPSPSSSKSPLPISRFSLPFSLNPNKSSSSSRRRGIKSSSPSSISAVLNTTTNVTTTPSPTKPTKPETFISRFAPDQPR

SEQ ID NO: 75 - protein, 573 aa, Arabidopsis thaliana
KGADILVEALERQGVETVFAYPGGASMEIHQALTRSSSIRNVLPRHEQGGVFAAEGYARSSGKPGICIATSGPGATNLVSGLADALLDSVPLVAITGQVPRRMIGTDAFQETPIVEVTRSITKHNYLVMDVEDIPRIIEEAFFLATSGRPGPVLVDVPKDIQQQLAIPNWEQAMRLPGYMSRMPKPPEDSHLEQIVRLISESKKPVLYVGGGCLNSSDELGRFVELTGIPVASTLMGLGSYPCDDELSLHMLGMHGTVYANYAVEHSDLLLAFGVRFDDRVTGKLEAFASRAKIVHIDIDSAEIGKNKTPHVSVCGDVKLALQGMNKVLENRAEELKLDFGVWRNELNVQKQKFPLSFKTFGEAIPPQYAIKVLDELTDGKAIISTGVGQHQMWAAQFYNYKKPRQWLSSGGLGAMGFGLPAAIGASVANPDAIVVDIDGDGSFIMNVQELATIRVENLPVKVLLLNNQHLGMVMQWEDRFYKANRAHTFLGDPAQEDEIFPNMLLFAAACGIPAARVTKKADLREAIQTMLDTPGPYLLDVICPHQEHVLPMIPSGGTFNDVITEGDGRIKY

SEQ ID NO: 76 - protein, 573 aa, Artificial Sequence; identical to SEQ ID NO: 75
  synthetic polypeptide

SEQ ID NO: 77 - DNA, 620 bp, Arabidopsis thaliana
ctgagataaacactcatttgtgaaagtttctaaactctgaatcgcgctacaggcaatgctccgaggagtaggaggagaagaacgaaccaaacgacattatcagccctttgaggaagctcttagttttgttattgtttttgtagccaaattctccattcttattccattttcacttatctcttgttccttatagaccttataagttttttattcatgtatacaaattatattgtcatcaagaagtatctttaaaatctaaatctcaaatcaccaggactatgtttttgtccaattcgtggaaccaacttgcagcttgtatccattctcttaaccaataaaaaaagaaagaaagatcaatttgataaatttctcagccacaaattctacatttaggttttagcatatcgaaggctcaatcacaaatacaatagatagactagagattccagcgtcacgtgagttttatctataaataaaggaccaaaaatcaaatcccgagggcattttcgtaatccaacataaaacccttaaacttcaagtctcatttttaaacaaatcatgttcacaagtctcttcttcttctctgtttctctatctcttgctcatctttctcctgaacc

SEQ ID NO: 78 - DNA, 500 bp, Arabidopsis thaliana
gagatgaaaccggtgattatcagaaccttttatggtctttgtatgcatatggtaaaaaaacttagtttgcaatttcctgtttgttttggtaatttgagtttcttttagttgttgatctgcctgctttttggtttacgtcagactactactgctgttgttgtttggtttcctttctttcattttataaataaataatccggttcggtttactccttgtgactggctcagtttggttattgcgaaatgcgaatggtaaattgagtaattgaaattcgttattagggttctaagctgttttaacagtcactgggttaatatctctcgaatcttgcatggaaaatgctcttaccattggtttttaattgaaatgtgctcatatgggccgtggtttccaaattaaataaaactacgatgtcatcgagaagtaaaatcaactgtgtccacattatcagttttgtgtatacgatgaaatagggtaattcaaaatctagcttgatatg

SEQ ID NO: 79 - DNA, 4135 bp, Artificial Sequence
  synthetic polynucleotide
catatcaagctagattttgaattaccctatttcatcgtatacacaaaactgataatgtggacacagttgattttacttctcgatgacatcgtagttttatttaatttggaaaccacggcccatatgagcacatttcaattaaaaaccaatggtaagagcattttccatgcaagattcgagagatattaacccagtgactgttaaaacagcttagaaccctaataacgaatttcaattactcaatttaccattcgcatttcgcaataaccaaactgagccagtcacaaggagtaaaccgaaccggattatttatttataaaatgaaagaaaggaaaccaaacaacaacagcagtagtagtctgacgtaaaccaaaaagcaggcagatcaacaactaaaagaaactcaaattaccaaaacaaacaggaaattgcaaactaagtttttttaccatatgcatacaaagaccataaaaggttctgataatcaccggtttcatctctcagtatttaatccggccatctccttccgttatgacatcgttgaaagtgccaccactcgggatcatcggcaacacatgttcttggtgcggacaaatcacatccaacaggtaaggtcctggtgtatccagcattgtctgaatagcttctcggagatctgctttctttgtcaccctcgccgctggaatcccgcaagctgctgcaaacagcaacatgttcgggaatatctcgtcctcctgagccggatccccgagaaatgtgtgagctcggttagctttgtagaaccgatcttcccattgcataaccatgccaagatgctggttgtttaataaaagtaccttcactggaagattctctacacgaatagtggctagctcttgcacattcattataaagcttccatctccgtcaatatccacaactatcgcatcagggttagcaacagacgctccaatcgcagcaggaagtccaaatcccatagctccaaggcctcctgatgatagccactgccttggtttcttgtaattgtagaactgcgccgcccacatttgatgttgcccgacaccagtacttattatggcttttccatcagtcaactcatcaaggaccttaatcgcatactgtggaggaatagcttccccaaacgtcttaaagctcaacggaaacttctgtttctgtacgttcaactcattcctccaaactccaaaatcaagcttaagctcctccgctcggttctcaagaaccttattcatcccttgcaaagccagcttaacatcaccacacacagacacatgaggagtcttattcttcccaatctcagccgagtcaatatcaatatgaacaatcttagccctactagcaaaagcctcaagcttacccgtgacacgatcatcaaaccttaccccaaacgccaacaacaaatcactatgctccacagcgtaatttgcatacacagtcccatgcattccaagcatatgtaacgacaactcatcatcacaaggataagatcccagccccatcaacgtactcgcaacagggatccccgtaagctcaacaaacctacccaattcatcgctagaattcaaacaaccaccaccaacatacaacacaggcttcttagactcagaaatcaacctaacaatctgctccaaatgagaatcttccggaggtttaggcatcctagacatataaccaggtaatctcatagcctgttcccaattaggaatcgcaagctgttgttgaatatctttaggaacatcaaccaaaacaggtccaggtctaccagaagtagctaaaaagaaagcttcctcaataatcctagggatatcttcaacatccatcacaagatagttatgcttcgtaatcgaacgcgttacctcaacaatcggagtctcttgaaacgcatctgtaccaatcatacgacgagagacttgtcctgtgattgctacaagaggaacactatctaacaacgcatcggctaatccgctaacgagatttgtagctccgggacctgaagtggctatacagatacctggtttacctgaggatcgagcgtatccttctgctgcgaatacacctccttgttcgtgacgaggaaggacgttacggattgaggaagagcgggttaaggcttggtgaatctccattgatgcacctccagggtaagcgaatacggtttctacgccttgacgttctaaagcttcgacgaggatatcagcgcctttgcggggttgatctggagcgaatcgggagatgaatgtttcgggtttggtaggtttggttggagagggagtggttgtgacattggtggttgtgttgagcacggcggagatggaggagggagagctggatttgataccgcggcggcgggaggaggaggatgatttgttggggtttagggagaatgggagggagaatctggagattggtaatggtgatttggaggaggaaggagatggtttggtggagaaggagatcgaagaagatgttgttgttgttgttgttgccgccgccatggttcaggagaaagatgagcaagagatagagaaacagagaagaagaagagacttgtgaacatgatttgtttaaaaatgagacttgaagtttaagggttttatgttggattacgaaaatgccctcgggatttgatttttggtcctttatttatagataaaactcacgtgacgctggaatctctagtctatctattgtatttgtgattgagccttcgatatgctaaaacctaaatgtagaatttgtggctgagaaatttatcaaattgatctttctttctttttttattggttaagagaatggatacaagctgcaagttggttccacgaattggacaaaaacatagtcctggtgatttgagatttagattttaaagatacttcttgatgacaatataatttgtatacatgaataaaaaacttataaggtctataaggaacaagagataagtgaaaatggaataagaatggagaatttggctacaaaaacaataacaaaactaagagcttcctcaaagggctgataatgtcgtttggttcgttcttctcctcctactcctcggagcattgcctgtagcgcgattcagagtttagaaactttcacaaatgagtgtttatctcagcagaagccaaatctttggtaaacaaagtaatttttttgccatttgattggttagtataggagaatttaaaaacgacgataaggtttaggtaaattatttcatttgaaaataattgagcaccgttaataattttcatccataaaataatatttcaaagatgatatttgatccccattaaattcattcgtaaccaaaaaaaagttatgaaaaaagagtggtcgtgtgagttgcccaagcaccattataataaaaaataaaataattagcaagtaataaggaataaaatcctgtaattatagctgaaaaaggaaaaatatttggagaccgtcagattcgaatctgaacaaagcataaaaaagtcaacaaaacttaaagcggcggtctcatcgtaatctcagcccaataccctattttcctctcccctatataaatactttcttcttctactgatcttcttctcacaaataaacccaaatatatcaatctactgtgttggtgattaagtactttgacaatccagccaatccagcagtggtcaaaaaagttgtagttttcttaaagtctctttcctctgctgattggcaggattgtcaaagagcttgctccctaaacttatctctctgatgatttaatgttagagatcttcgtaaatctatgtgtttgatagatctgatgcgttttttgagttgatgatttgattatttttcactggaaagtatctcattagggtaacgataatgttttatggatttggttgtataacagatccatgaaatcttgactggttataaaatctgaataatgtatttcaatttggagattcggtgataaaaattactgatttacgaatgacatttatatcgatagatgagtttgctgatttggttgttaaattgataaatcaaggacatgagaaactgtttttgtatgctaatttgtccatggaataaaattgggaggtgaggaccgtgagggtagtcaggaaaccttaatattgaagtt

SEQ ID NO: 80 - DNA, 4198 bp, Artificial Sequence
  synthetic polynucleotide
gcatatcgaattttatacttaattcaaagccaatggaactaacacatgatagatatcaagaggaggctgaaaaaaagaggtaaatgatcttgtccatctgtaacagaacactactaatcgaaacgtaagacctattaaaggaggaaatgtgcacccctaccaaagttctagttacacgttcaagtgtttccatatattctagttacacaagctattatcctcgtgaccgtttaaaccacccattttcaaatagagctccagataatgaaataggtgctgccaataaaaaataataatctgaatccttccattcgacaaaagcaagatgatgcaaaactttaagtgaacataaataccaagtagaagatagtacaacagccacaacaagcaaagccacgccaaagactaacagcaaactagacaaatcgaacacttttggcttaatagaagtttattttagataatacaatgtctagaactagttatgtagtttctttaagtcaataggaacatctcccatcgccctccgtgatcacatctttgaaagcaccaccgctgggaatcataggtagaacgtgctcctgatgaggtacaatcacatccaacaagtatggcccaggagtgtctaacatcttttgaatggcagctctaagatcatccctgtgtgacactcttgcagcaggtacgccacaagcctctgcaaatttcaacatattagggaagatctcttcctcgttagaagggtcacccaagtaagtgtgtgctctgttagctttatagaatcgatcctcccattgaaccaccattcccaagtgttgattattcagcaacataatcttaactgggagattctccaccttaattgttgctaactcttgcacattcatgataaaactcccatcaccgtcaatgtcaaccacaatctcacccggtctcccaacagccgcacctatagcagcaggcaaaccaaatcccattgctcctaatccaccagatgtcaaccattggcgtggctttttgtacttatagtattgggcagcccacatctggtgttgccccacaccagtactaataatggcatttccgttagttaactcatcaagaacctgaatagcatattgtggagggatggcatcaccaaaagtcttaaaattcagtgggtacttcatcttctgctccgttaactcctgcctccaggcagaaaaatctaacttcatcttaccttctttaccctccaatatggaattcaaaccctgtaatgccaacttgatatctgcacaaatggaaacatgaggttgcttgttttttccaatctctgccgaatcaatatcaatgtggacaattttcgctcgactagcaaaagcttccaatttaccagtaactcgatcatcaaacctcaccccaaatgcaagcaacaaatcactactatccacagcataattagcatacacagttccatgcatacccaacatttgaagtgaaagctcatccccagttggaaaagctccaagacccatcaaagtactcgctacaggaatacctgtaagctccacaaatcgtctcagctcctcacttgattgcgaacacccaccacccacatacaaaacaggcttcttcgactcggaaatcagcctaacaatttgttccaaaagcatttcattaggcaatttaggtaacctagacatgtaaccaggcaacctcattggctgatcccaattaggtatcaccaattgttgctgaatatccttaggtacatcaatcaaaactgggccaggccgtcccgatttcgcaagaaaaaatgcttcacgaacaaccctaggaatatcttctacatccataacaagataattatgcttcgtaatagatctcgttacctcaacaataggcgtttcctggaacgcatcagtaccaatcatcctccttgccacttgacctgtaatagcaacaatcggaatactatctaacaaagcatccgcaagaccactaacaagatttgtagctccgggaccagaggtagcaatgcaaacaccagggaacccagtagcccgtgcgtaaccctctgcagcaaacacaccaccttgctcatgacgtggtagcacattacgaataatattcgaacgtgtcaaagcttgatgaatctccatagaagcacctcctgggtatgcaaatacatccgtaacaccttccctttcaagtgcctccacaagaacatcacaaccctttctgggttcgtcaggggcgaaacgggaaacgaatgtttcaggttcagaaacgtcgttatgggtggtagtggatatgacgacattggcaacggcaaaaccacgacgattatgttgagcgtggatgagatgaaggggtgaggctttttgtgggtgattgtggaaagagaaggtagatctaggtagaatggtggaagatttggaggaagatggaggtagggttttggagaaacatggagatggtgaggcagcagccgccattgttggtcgataaaggtgagagaagtgaaaggaagaggcaaaatggctataagaagtgatgagcgccacgaaaatgtgttgtaagggtcctacattgattcaaatataaatggaaaacaatttataagttcaaaatcaaaaatgtcgtaaatgatactatatcctttgtaatattgatgttattatggttcaaaaattacgttacatcctttcgctttaacgaaagattaaatagaaatatgtgatataatattatatattaatttaatatttaataaaaaataaattgataaataaaattataatatgtatatatgtaaaaataaaaattatgtgtattctaatatttaaataataaaaatattaatatccaaaaatatgattaaaaatatctaaatgacatttatgataattaagaaatatatttatgattggaaattatgcataatctcacatcacctttcaatatgagtcctattttaaaaaataatattttcattaaaaaaaaaaataataatcatctatagtacttgtgaattttttttccctctgttttcaaatctcttttatcccatcaattctttttttttttgggtctagtaaattgtagtagagtatatagtattgtgtggtaaatgttataaagagtggggggtgggatgggatgggatgggatggtgtttttaattttttgcaaagtgatggatagactttttactccacagcatcaattggtattgaaaaatcaaaatcaatgagccaaaaagacaattgtgatagccccttttcacttgtttacttgattgcttcacctatggggtctttaaagatttgcccactccaagactccaagctttaactcttcttgtccctctttactttaattctatccatctttttatttattcaccacccccacccctacccacatccccacccctaccacatcctcacatctttttccagctataaatacttgagctaccccaatcttcttatttgtcacctcaagttcaaaaatgaacaattcactcacacaatgcaagaaaataccttttttctttttttaaagttgtagttaactttaatggaaatgttggcaataatgggtgggtaactcatttgggatggtagttgcgattgcaacttcttgtacacgacaacatatgcattactaagggtaatgcatgaatgttgtaatcgtagcgaccgttccggccgaatttcctacctcctctcttctcaagattcttcaaaattcaaaccacttttgacctaatgtgccttgagacgtaattgttgtacattacttttttaaagtatacgctataataccaaggaaacattaacgactaaactaatggcatgcatgactatgcccttgactaaagagtagtgcaaattattttcactatattatttacaactctcttttttgcagggtgtctatagagaattgacagttgagttttctatttatataacataaattttaatgtttttgtcatgctgttgtttttaggtggcaatgaagggagatggaccaaacatggagctagtggtccaatgctgtctaggctatagcttacctacccacagagttctgcttcaacttaatatggcatttgacatcttgtacttaaattgtgtaataaaaaaaaattacatcttgtacttatagtataagaaaaaaaaaattacttttactctctaatcta

SEQ ID NO: 81 - DNA, 2290 bp, Arabidopsis thaliana
ctttggtgggcaaaaacatattagctgagaggtcaatttctttttcccctaaaccaaattacgttgagatgcatggtctctctctactcaattaaccaaataaggaaaagaatcatatggtcatcaattcgtaaatcaaaattttaatttgtgtggtatttaatccatctacatgtttcgtaagcaacaaaagagcttggtctgaaaaccaaacaagaccatatgggcactcgaatactccattttgttatcggctacttccactagcctcctccttcgctgcgtctcctgtttctctacttcacgattactcgctagattcattgaagcagttgtgagttaagttggagaaaatggttgtgtttgggaatgtttctgcggcgaatttgccttatcaaaacgggtttttggaggcactttcatctggaggttgtgaactaatgggacatagctttagggttcccacttctcaagcgcttaagacaagaacaaggaggaggagtactgctggtcctttgcaggtagtttgtgtggatattccaaggccagagctagagaacactgtcaatttcttggaagctgctagtttatctgcatccttccgtagtgctcctcgtcctgctaagcctttgaaagttgtaattgctggtgctggattggctggattgtcaactgcaaagtacctggctgatgcaggccacaaacctctgttgcttgaagcaagagatgttcttggtggaaagatagctgcatggaaggatgaagatggggactggtatgagactggtttacatattttcttcggtgcttatccgaatgtgcagaatttatttggagaacttgggatcaatgatcggttgcagtggaaggaacactccatgatttttgctatgccaagtaaacctggagaatttagtagatttgacttcccagatgtcctaccagcacccttaaatggtatttgggctattttgcggaacaacgagatgctgacatggccagagaaaataaagtttgctattggacttttgccagccatggtcggcggtcaggcttatgttgaggcccaagatggtttatcagtcaaagaatggatggaaaagcagggagtacctgagcgcgtgaccgacgaggtgtttattgccatgtcaaaggcgctaaactttataaaccctgatgaactgtcaatgcaatgcattttgatagctttgaaccggtttcttcaggaaaaacatggttccaagatggcattcttggatggtaatcctccggaaaggctttgtatgccagtagtggatcatattcgatcactaggtggggaagtgcaacttaattctaggataaagaaaattgagctcaatgacgatggcacggttaagagtttcttactcactaatggaagcactgtcgaaggagacgcttatgtgtttgccgctccagtcgatatcctgaagctccttttaccagatccctggaaagaaataccgtacttcaagaaattggataaattagttggagtaccagttattaatgttcatatatggtttgatcgaaaactgaagaacacatatgatcacctactctttagcagaagtaaccttctgagcgtgtatgccgacatgtccttaacttgtaaggaatattacgatcctaaccggtcaatgctggagctagtatttgcaccagcagaggaatggatatcacggactgattctgacatcatagatgcaacaatgaaagaacttgagaaactcttccctgatgaaatctcagctgaccaaagcaaagctaaaattctgaagtaccatgtcgttaagactccaagatctgtgtacaagaccatcccaaactgtgaaccatgtcgtcctctacaaagatcacctattgaaggattctacttagctggagattacacaaaacagaagtacttagcttccatggaaggcgctgtcctctctggcaaattctgctctcagtctattgttcaggattacgagctactggctgcgtctggaccaagaaagttgtcggaggcaacagtatcatcatcatgagaagaggacaaaacttaaagatgatttgcttgtaagcattattatttgtgtataaatctcattgcaatccaaacttaaccttactctcttcagtaaatgaatctcacagatttgacatctcacgtttctgtcaattttataatttttaaaaagtaattactgtcgaccttttgtaatcatagtgatttatcattatgtctctctttttaaaaccttttctggtacaaattataaaa

SEQ ID NO: 82 - DNA, 4135 bp, Artificial Sequence
  synthetic polynucleotide
catatcaagctagattttgaattaccctatttcatcgtatacacaaaactgataatgtggacacagttgattttacttctcgatgacatcgtagttttatttaatttggaaaccacggcccatatgagcacatttcaattaaaaaccaatggtaagagcattttccatgcaagattcgagagatattaacccagtgactgttaaaacagcttagaaccctaataacgaatttcaattactcaatttaccattcgcatttcgcaataaccaaactgagccagtcacaaggagtaaaccgaaccggattatttatttataaaatgaaagaaaggaaaccaaacaacaacagcagtagtagtctgacgtaaaccaaaaagcaggcagatcaacaactaaaagaaactcaaattaccaaaacaaacaggaaattgcaaactaagtttttttaccatatgcatacaaagaccataaaaggttctgataatcaccggtttcatctctcagtatttaatccggccatctccttccgttatgacatcgttgaaagtgccaccactcgggatcatcggcaacacatgttcttggtgcggacaaatcacatccaacaggtaaggtcctggtgtatccagcattgtctgaatagcttctcggagatctgctttctttgtcaccctcgccgctggaatcccgcaagctgctgcaaacagcaacatgttcgggaatatctcgtcctcctgagccggatccccgagaaatgtgtgagctcggttagctttgtagaaccgatcttcccattgcataaccatgccaagatgctggttgtttaataaaagtaccttcactggaagattctctacacgaatagtggctagctcttgcacattcattataaagcttccatctccgtcaatatccacaactatcgcatcagggttagcaacagacgctccaatcgcagcaggaagtccaaatcccatagctccaaggcctcctgatgatagccactgccttggtttcttgtaattgtagaactgcgccgcccacatttgatgttgcccgacaccagtacttattatggcttttccatcagtcaactcatcaaggaccttaatcgcatactgtggaggaatagcttccccaaacgtcttaaagctcaacggaaacttctgtttctgtacgttcaactcattcctccaaactccaaaatcaagcttaagctcctccgctcggttctcaagaaccttattcatcccttgcaaagccagcttaacatcaccacacacagacacatgaggagtcttattcttcccaatctcagccgagtcaatatcaatatgaacaatcttagccctactagcaaaagcctcaagcttacccgtgacacgatcatcaaaccttaccccaaacgccaacaacaaatcactatgctccacagcgtaatttgcatacacagtcccatgcattccaagcatatgtaacgacaactcatcatcacaaggataagatcccagccccatcaacgtactcgcaacagggatccccgtaagctcaacaaacctacccaattcatcgctagaattcaaacaaccaccaccaacatacaacacaggcttcttagactcagaaatcaacctaacaatctgctccaaatgagaatcttccggaggtttaggcatcctagacatataaccaggtaatctcatagcctgttcccaattaggaatcgcaagctgttgttgaatatctttaggaacatcaaccaaaacaggtccaggtctaccagaagtagctaaaaagaaagcttcctcaataatcctagggatatcttcaacatccatcacaagatagttatgcttcgtaatcgaacgcgttacctcaacaatcggagtctcttgaaacgcatctgtaccaatcatacgacgagagacttgtcctgtgattgctacaagaggaacactatctaacaacgcatcggctaatccgctaacgagatttgtagctccgggacctgaagtggctatacagatacctggtttacctgaggatcgagcgtatccttctgctgcgaatacacctccttgttcgtgacgaggaaggacgttacggattgaggaagagcgggttaaggcttggtgaatctccattgatgcacctccagggtaagcgaatacggtttctacgccttgacgttctaaagcttcgacgaggatatcagcgcctttgcggggttgatctggagcgaatcgggagatgaatgtttcgggtttggtaggtttggttggagagggagtggttgtgacattggtggttgtgttgagcacggcggagatggaggagggagagctggatttgataccgcggcggcgggaggaggaggatgatttgttggggtttagggagaatgggagggagaatctggagattggtaatggtgatttggaggaggaaggagatggtttggtggagaaggagatcgaagaagatgttgttgttgttgttgttgccgccgccatggttcaggagaaagatgagcaagagatagagaaacagagaagaagaagagacttgtgaacatgatttgtttaaaaatgagacttgaagtttaagggttttatgttggattacgaaaatgccctcgggatttgatttttggtcctttatttatagataaaactcacgtgacgctggaatctctagtctatctattgtatttgtgattgagccttcgatatgctaaaacctaaatgtagaatttgtggctgagaaatttatcaaattgatctttctttctttttttattggttaagagaatggatacaagctgcaagttggttccacgaattggacaaaaacatagtcctggtgatttgagatttagattttaaagatacttcttgatgacaatataatttgtatacatgaataaaaaacttataaggtctataaggaacaagagataagtgaaaatggaataagaatggagaatttggctacaaaaacaataacaaaactaagagcttcctcaaagggctgataatgtcgtttggttcgttcttctcctcctactcctcggagcattgcctgtagcgcgattcagagtttagaaactttcacaaatgagtgtttatctcagcagaagccaaatctttggtaaacaaagtaatttttttgccatttgattggttagtataggagaatttaaaaacgacgataaggtttaggtaaattatttcatttgaaaataattgagcaccgttaataattttcatccataaaataatatttcaaagatgatatttgatccccattaaattcattcgtaaccaaaaaaaagttatgaaaaaagagtggtcgtgtgagttgcccaagcaccattataataaaaaataaaataattagcaagtaataaggaataaaatcctgtaattatagctgaaaaaggaaaaatatttggagaccgtcagattcgaatctgaacaaagcataaaaaagtcaacaaaacttaaagcggcggtctcatcgtaatctcagcccaataccctattttcctctcccctatataaatactttcttcttctactgatcttcttctcacaaataaacccaaatatatcaatctactgtgttggtgattaagtactttcgcttgcagagagaaatcacagtggtcaaaaaagttgtagttttcttaaagtctctttcctctgtgattctctgtgtaagcgaaagagcttgctccctaaacttatctctctgatgatttaatgttagagatcttcgtaaatctatgtgtttgatagatctgatgcgttttttgagttgatgatttgattatttttcactggaaagtatctcattagggtaacgataatgttttatggatttggttgtataacagatccatgaaatcttgactggttataaaatctgaataatgtatttcaatttggagattcggtgataaaaattactgatttacgaatgacatttatatcgatagatgagtttgctgatttggttgttaaattgataaatcaaggacatgagaaactgtttttgtatgctaatttgtccatggaataaaattgggaggtgaggaccgtgagggtagtcaggaaaccttaatattgaagtt

SEQ ID NO: 83 - DNA, 1200 bp, Artificial Sequence
  synthetic polynucleotide
aggatagaaaatttcagatgatggtgaaaacttagaggagtagttctctggtatagtgaccaatgcataagtcgaatcatttgggtaatctagttctgaaatcttaggatgccatatgcggtggtaatatgtgatgtttccgggtacggaaatttctttggaactaagaattagagtgagtgaagtcacatgaacattaatgtagatcactgatcatctggtggatatcaaaacaatcaaccacaaaacaagaaaacaaatgtacgtagaattccaagaccattagtgattacgatatattaagcttacattgcatcttagaaggcaattcaaggtcatcatgcatgttcaaagaagataaacaatgatcctttacctcttgtgtgaagcaaagtttgtttaacaattctctaggcatctctaatggaataaaactaagaggaccaccgagttcagtttccagaagcttcgtacgtagctttctccacgaatcagatggtgttaagtatcaacttcgcaccaagagaggccagccttctattatttcaaagtagttgctgcacgtatactccacgagatgctgtttcgaaaaacaatagagacctctaacttcggaacggcagacaaaggagtagacacacggaggctagtttacatagttgaagggaagcctctcaaaggcacactgaaggtacttaaaaccatatgataccaaggagaaatcaatgaaacttttagagactgaatgatgttgttatcttagttgtcttccactagagatgcataatggaatcgttaataaaatttgccccgcacaaaacgaaggattattgctcttatgctttaaacatgtatataatattacactaccttttaagatgcgaggtaagctaaataaatactagtgattaagaaatattacaactgtcttcttcttgcgttgctacttgattcgaccacatgatcgctacaaggatgttcactcaatctatttgttgatgacgaagtatcttcctcaccagatgatggggtcatcaagtcagcatcaaccaaggatcatggaggaggcaccaataagtcgaccaagatccacgagagaaccaacctaagcataccaaatacatatgtgcctttcaagaagcataccagcccaacccgaagaatttaagctacaaatcaacttattccaaaaacttaact

SEQ ID NO: 84 - DNA, 1200 bp, Artificial Sequence
  synthetic polynucleotide
aggatagaaaatttcagatgatggtgaaaacttagaggagtagttctctggtatagtgaccaatgcataagtcgaatcatttgggtaatctagttctgaaatcttaggatgccatatgcggtggtaatatgtgatgtttccgggtacggaaatttctttggaactaagaattagagtgagtgaagtcacatgaacattaatgtagatcactgatcatctggtggatatcaaaacaatcaaccacaaaacaagaaaacaaatgtacgtagaattccaagaccattagtgattacgatatattaagcttacattgcatcttagaaggcaattcaaggtcatcatgcatgttcaaagaagataaacaatgatcctttacctcttgtgtgaagcaaagtttgtttaacaattctctaggcatctctaatggaataaaactaagaggaccaccgagttcagtttccagaagcttcgtacgtagctttctccacgaattgggaggcggtaggtacggatttctcaccgatggtaaccagacagctttcgtggggttcactaatctgcacgtacactctacgaaattcttttgcggtaagcaacagggacacctgacaccgcaaaggaagacaaaagagtggacatacggtttagtcggcggatgagtgttgtgaagttgccaaaaggcacaaagaaagtacctacagcctcacgataccaaggagaaatcaatgaaacttttagagactgaatgatgttgttatcttagttgtcttccactagagatgcataatggaatcgttaataaaatttgccccgcacaaaacgaaggattattgctcttatgctttaaacatgtatataatattacactaccttttaagatgcgaggtaagctaaataaatactagtgattaagaaatattacaactgtcttcttcttgcgttgctacttgattcgaccacatgatcgctacaaggatgttcactcaatctatttgttgatgacgaagtatcttcctcaccagatgatggggtcatcaagtcagcatcaaccaaggatcatggaggaggcaccaataagtcgaccaagatccacgagagaaccaacctaagcataccaaatacatatgtgcctttcaagaagcataccagcccaacccgaagaatttaagctacaaatcaacttattccaaaaacttaact

SEQ ID NO: 85 - DNA, 1200 bp, Artificial Sequence
  synthetic polynucleotide
gccaacttctatctcacctacaaatgtataaagggcattgttacaagaacacaggaaaatttgtaccgatttatacaatggcaaatttgtaccgatttgtacctacaaatgtaccgatttataaagagaaatcttgcatgttggaaaaaagcaagaaaattttgaagctagactttttgatacatgaaacaaaacacaacactttttcaggaacattgtttggtcagacgaagtaagcaacctacaagaatctgaacttctttttaattatccaacgccaaatctacttgataaacattaagataaagctaaggcgcttatcagtcaactacacacttcaataacaagcacttcattaactgaaaaaacaaacaaactaaagaggtcgaattctcaccagaaacaacagtcacctcttcatctgctctctaagaacacaaaatcctcagctttcccagaaaagtaatcggacttgactctgttttccctcacttttttcttctttcaaaaaccaataaacaaaaagattccagaaactgagaacgatacccggggtcgtcgcttcagcgctaatattgtagaccttttcttgcttttacctctttgaagtgttcacaggaagaagcctactacatgaacaataaagcggcccaacctcttctcgcttcttaactccaaaatgtcaacgaaacataaattttccgaatcggatcaaacgaaacacatcagatctgaaaattcaacagtcaaaatctataccacaagtagtaaatagctggatctttattgccttatgaaaccaaatggacaaaaacatataccctagattacacacatgcatctgcagacaaactgggagaaagagaatcagtgtgatgatagagatatgtcaaattaatgaatccaacaaagaaagaaaaaggaaacagagagatgaaagttttaaccaggaatcttcttgaccagataccatttgtgattctctgatttgcctattggcctactacatccctcctctctctctatcttttaataacaaactttatagatctggttttttcttctagaactaaaatgtatttgattcatatttataactaaaatgtatctaaaagtctcataacatatttgtccaagaaaaaattgtagtaacatagttattgaatttgaatatcataagatttaggaagacattttaaa

SEQ ID NO: 86 - DNA, 1200 bp, Artificial Sequence
  synthetic polynucleotide
gccaacttctatctcacctacaaatgtataaagggcattgttacaagaacacaggaaaatttgtaccgatttatacaatggcaaatttgtaccgatttgtacctacaaatgtaccgatttataaagagaaatcttgcatgttggaaaaaagcaagaaaattttgaagctagactttttgatacatgaaacaaaacacaacactttttcaggaacattgtttggtcagacgaagtaagcaacctacaagaatctgaacttctttttaattatccaacgccaaatctacttgataaacattaagataaagctaaggcgcttatcagtcaactacacacttcaataacaagcacttcattaactgaaaaaacaaacaaactaaagaggtcgaattctcaccagaaacaacagtcacctcttcatctgctctctaagaacacaaaatcctcagctttcccagaaaagtaatcggacttgactctgttttccctcacttttttcttctttcaaaaaccaataaacaaaaagattccagaaaatacgtcagatacgcgaggcttacgcttccttgggccccattaacctcagctcttgcttttacaattttattgtgttcgcaggaggcgagattagtgaaccccacgaaagcgactcactctcaacgtccttcttaactccaaaatgtcaacgaaacataaattttccgaatcggatcaaacgaaacacatcagatctgaaaattcaacagtcaaaatctataccacaagtagtaaatagctggatctttattgccttatgaaaccaaatggacaaaaacatataccctagattacacacatgcatctgcagacaaactgggagaaagagaatcagtgtgatgatagagatatgtcaaattaatgaatccaacaaagaaagaaaaaggaaacagagagatgaaagttttaaccaggaatcttcttgaccagataccatttgtgattctctgatttgcctattggcctactacatccctcctctctctctatcttttaataacaaactttatagatctggttttttcttctagaactaaaatgtatttgattcatatttataactaaaatgtatctaaaagtctcataacatatttgtccaagaaaaaattgtagtaacatagttattgaatttgaatatcataagatttaggaagacattttaaa

SEQ ID NO: 87 - DNA, 1200 bp, Artificial Sequence
  synthetic polynucleotide
gtaaaaaaacatgtgacgacgaaattcaatcagttcctccaatgtttggcagaagccaaatctttggtaaacaaagtaatttttttgccatttgattggttagtataggagaatttaaaaacgacgataaggtttaggtaaattatttcatttgaaaataattgagcaccgttaataattttcatccataaaataatatttcaaagatgatatttgatccccattaaattcattcgtaaccaaaaaaaagttatgaaaaaagagtggtcgtgtgagttgcccaagcaccattataataaaaaataaaataattagcaagtaataaggaataaaatcctgtaattatagctgaaaaaggaaaaatatttggagaccgtcagattcgaatctgaacaaagcataaaaaagtcaacaaaacttaaagcggcggtctcatcgtaatctcagcccaataccctattttcctctcccctatataaatactttcttcttctactgatcttcttctcacaaataaacccaaatatatcaatctactgtgttggtgataaagcactttacatgttgatgaacttcctgagcggtcaaaaaagtggcgaaatatctcaagcaaggttccacttaggggttcagcggtatgtaaagagctttctccctaaacttatctctctgatgatttaatgttagagatcttcgtaaatctatgtgtttgatagatctgatgcgttttttgagttgatgatttgattatttttcactggaaagtatctcattagggtaacgataatgttttatggatttggttgtataacagatccatgaaatcttgactggttataaaatctgaataatgtatttcaatttggagattcggtgataaaaattactgatttacgaatgacatttatatcgatagatgagtttgctgatttggttgttaaattgataaatcaaggacatgagaaactgtttttgtatgctaatttgtccatggaataaaattgggaggtgaggaccgtgagggtagtcaggaaaccttaatattgaagttgatgttgaaccaacaaatctgcccaaaaatgataaaagttgatgccgagcccacaaattttgatgacaatcgataagcccaagcccaaaaggcatctgtacctgagcccattattctttcattactagcaaaaaggatgcattagagac

SEQ ID NO: 88 - DNA, 1200 bp, Artificial Sequence
  synthetic polynucleotide
gtaaaaaaacatgtgacgacgaaattcaatcagttcctccaatgtttggcagaagccaaatctttggtaaacaaagtaatttttttgccatttgattggttagtataggagaatttaaaaacgacgataaggtttaggtaaattatttcatttgaaaataattgagcaccgttaataattttcatccataaaataatatttcaaagatgatatttgatccccattaaattcattcgtaaccaaaaaaaagttatgaaaaaagagtggtcgtgtgagttgcccaagcaccattataataaaaaataaaataattagcaagtaataaggaataaaatcctgtaattatagctgaaaaaggaaaaatatttggagaccgtcagattcgaatctgaacaaagcataaaaaagtcaacaaaacttaaagcggcggtctcatcgtaatctcagcccaataccctattttcctctcccctatataaatactttcttcttctactgatcttcttctcacaaataaacccaaatatatcaatctactgtgttggtgataaggaacatttattgttcatgtagtaggctcggggtcaaaaaagtcggaggatgcttgaagtcgcaatccgcgggccactacagggatagtgaatgatccttctccctaaacttatctctctgatgatttaatgttagagatcttcgtaaatctatgtgtttgatagatctgatgcgttttttgagttgatgatttgattatttttcactggaaagtatctcattagggtaacgataatgttttatggatttggttgtataacagatccatgaaatcttgactggttataaaatctgaataatgtatttcaatttggagattcggtgataaaaattactgatttacgaatgacatttatatcgatagatgagtttgctgatttggttgttaaattgataaatcaaggacatgagaaactgtttttgtatgctaatttgtccatggaataaaattgggaggtgaggaccgtgagggtagtcaggaaaccttaatattgaagttgatgttgaaccaacaaatctgcccaaaaatgataaaagttgatgccgagcccacaaattttgatgacaatcgataagcccaagcccaaaaggcatctgtacctgagcccattattctttcattactagcaaaaaggatgcattagagac

SEQ ID NO: 89 - DNA, 1200 bp, Artificial Sequence
  synthetic polynucleotide
gtaaaaaaacatgtgacgacgaaattcaatcagttcctccaatgtttggcagaagccaaatctttggtaaacaaagtaatttttttgccatttgattggttagtataggagaatttaaaaacgacgataaggtttaggtaaattatttcatttgaaaataattgagcaccgttaataattttcatccataaaataatatttcaaagatgatatttgatccccattaaattcattcgtaaccaaaaaaaagttatgaaaaaagagtggtcgtgtgagttgcccaagcaccattataataaaaaataaaataattagcaagtaataaggaataaaatcctgtaattatagctgaaaaaggaaaaatatttggagaccgtcagattcgaatctgaacaaagcataaaaaagtcaacaaaacttaaagcggcggtctcatcgtaatctcagcccaataccctattttcctctcccctatataaatactttcttcttctactgatcttcttctcacaaataaacccaaatatatcaatctactgtgttggtgataaagcacttttcgtggggttcactaatctctgcggacaaaaaagattcacatcggtaatacccacaagccccagagatagtggtttccacggaaagagctttctccctaaacttatctctctgatgatttaatgttagagatcttcgtaaatctatgtgtttgatagatctgatgcgttttttgagttgatgatttgattatttttcactggaaagtatctcattagggtaacgataatgttttatggatttggttgtataacagatccatgaaatcttgactggttataaaatctgaataatgtatttcaatttggagattcggtgataaaaattactgatttacgaatgacatttatatcgatagatgagtttgctgatttggttgttaaattgataaatcaaggacatgagaaactgtttttgtatgctaatttgtccatggaataaaattgggaggtgaggaccgtgagggtagtcaggaaaccttaatattgaagttgatgttgaaccaacaaatctgcccaaaaatgataaaagttgatgccgagcccacaaattttgatgacaatcgataagcccaagcccaaaaggcatctgtacctgagcccattattctttcattactagcaaaaaggatgcattagagac